SEQUENCE LISTING (16 sequences)

SEQ ID NO: 1 - DNA, 21 bp, Artificial Sequence
  Synthetic Polynucleotide
ccgcttgaaggaataattaaa

SEQ ID NO: 2 - DNA, 21 bp, Artificial Sequence
  Synthetic Polynucleotide
ccgcttgaaggaaaaattaaa

SEQ ID NO: 3 - DNA, 23 bp, Artificial Sequence
  Synthetic Polynucleotide
ccgcttgaagtcttgtaattaaa

SEQ ID NO: 4 - DNA, 21 bp, Artificial Sequence
  Synthetic Polynucleotide
ccgcttgaagaaataattaaa

SEQ ID NO: 5 - DNA, 22 bp, Artificial Sequence
  Synthetic Polynucleotide
ccgcttgaagagaataattaaa

SEQ ID NO: 6 - DNA, 22 bp, Artificial Sequence
  Synthetic Polynucleotide
ccgcttgaagagattaattaaa

SEQ ID NO: 7 - DNA, 21 bp, Artificial Sequence
  Synthetic Polynucleotide
ccgcttgaaggaaatattaaa

SEQ ID NO: 8 - DNA, 23 bp, Artificial Sequence
  Synthetic Polynucleotide
ccgcttgaaggtctttaattaaa

SEQ ID NO: 9 - DNA, 21 bp, Artificial Sequence
  Synthetic Polynucleotide
ccggttgaaggaataattaaa

SEQ ID NO: 10 - DNA, 21 bp, Artificial Sequence
  Synthetic Polynucleotide
ccgcttgaaagaaaaattaaa

SEQ ID NO: 11 - DNA, 21 bp, Artificial Sequence
  Synthetic Polynucleotide
ccgcttgaaagaataattaaa

SEQ ID NO: 12 - DNA, 25 bp, Artificial Sequence
  Synthetic Polynucleotide
ccgcttgaaggggtctttaattaaa

SEQ ID NO: 13 - DNA, 22 bp, Artificial Sequence
  Synthetic Polynucleotide
ccgcttgaagagtttaattaaa

SEQ ID NO: 14 - DNA, 22 bp, Artificial Sequence
  Synthetic Polynucleotide
ccggttgaagagtttaattaaa

SEQ ID NO: 15 - DNA, 21 bp, Artificial Sequence
  Synthetic Polynucleotide
ccgcttgacagtttaattaaa

SEQ ID NO: 16 - DNA, 22 bp, Artificial Sequence
  Synthetic Polynucleotide
ccgcttgaagtctttaattaaa

(19) United States
(12) Patent Application Publication
(10) Pub. No.: US 2019/0002912 A1
(43) Pub. Date: Jan. 3, 2019

(54) TUMOR IMMUNOTHERAPY

(71) Applicant: Massachusetts Institute of Technology, Cambridge, MA (US)

(72) Inventors: Timothy Kuan-Ta Lu, Cambridge, MA (US); Lior Nissim, Cambridge, MA (US); Ming-Ru Wu, Brookline, MA (US)

(73) Assignee: Massachusetts Institute of Technology, Cambridge, MA (US)

(21) Appl. No.: 15/737,829

(22) PCT Filed: Jun. 17, 2016
(86) PCT No.: PCT/US16/38222

§ 371 Date: Dec. 19, 2017

Related U.S. Application Data
(60) Provisional application No. 62/325,314, filed on Apr. 20, 2016.
(60) Provisional application No. 62/181,906, filed on Jun. 19, 2015.

(51) Int. Cl. C12N 15/85; C07K 14/47; C07K 16/32; C07K 16/28

(52) U.S. Cl. CPC C12N 15/85; C07K 14/4702; C07K 16/32; C07K 2319/03; C12N 2830/002; C07K 2317/622; C07K 16/2809

(57) ABSTRACT

Aspects of the present disclosure provide a platform that triggers potent and effective immunotherapy against tumors from within tumors themselves, thus overcoming limitations of existing cancer immunotherapies and tumor-detecting gene circuits.

### RELATED APPLICATIONS

This application claims the benefit under 35 U.S.C. § 119(e) of U.S. provisional application No. 62/181,906, filed Jun. 19, 2015, and U.S. provisional application No. 62/325,314, filed Apr. 20, 2016, each of which is incorporated by reference herein in its entirety.

### FIELD OF THE INVENTION

Aspects of the present disclosure relate to the general field of biotechnology and, more particularly, to the fields of synthetic biology and immunology.

### BACKGROUND

Existing treatments for many cancers (e.g., ovarian cancer), such as chemotherapies and targeted therapies, are unable to cure metastatic disease and prevent tumor relapse. Further, standard-of-care treatments, such as chemotherapy, can cause significant morbidity and toxicity. New therapeutic strategies are needed to treat primary and metastatic ovarian cancer and to achieve long-term efficacy.

### SUMMARY

Provided herein, in some aspects, is a platform that triggers potent and effective immunotherapy against tumors from within tumors themselves, thus overcoming limitations of existing cancer immunotherapies and tumor-detecting gene circuits. Engineered genetic circuits of the present disclosure, in some embodiments, express T-cell-engaging proteins on cancer cell surfaces (referred to as Surface T Cell Engagers (STEs)), which can trigger antigen-independent T cell killing of tumor cells. In some embodiments, engineered genetic circuits are delivered to tumors (see, e.g., FIGS. 2A and 2B), and are selectively activated only in cancer cells, resulting in the surface display of STEs and the secretion of other immunomodulatory molecules to recruit T cells to target the tumor. The engineered genetic circuits of the present disclosure, advantageously, can be administered systemically but activated locally only in cancer cells, resulting in enhanced safety and reduced side effects. Thus, the platform of the present disclosure, in some embodiments, combines the advantages of systemic delivery (e.g., treating metastasis) with the advantages of localized treatment (e.g., safety, minimal side effects).

Existing therapies are hindered by certain limitations that are overcome by the present disclosure. In CAR (chimeric antigen receptor) T cell therapy, for example, the T cells must be custom made for each individual. As another example, bispecific T cell engagers (BiTEs) (Iwahori K. et al., Molecular Therapy, 2015, 23(1): 171-178, incorporated herein by reference) are limited by their short half-life, and thus require a continuous intravenous pump infusion for 4-8 weeks. Both therapies target tumor cell surface antigens; however, not all tumor types have ideal surface tumor antigens for detection. Cancer-detecting genetic circuits can harness an intracellular killing mechanism, inducing cell death via a toxin, although delivery of these circuits to all (or most) tumor cells has been virtually impossible.

The present disclosure, in some aspects, provides methods and engineered (recombinant or synthetic) genetic circuits (e.g., engineered mammalian genetic circuits), referred to in some embodiments as “logic gates” that are RNA-based (e.g., the genetic circuits include nucleic acids that comprise primarily RNA, or the genetic include nucleic acids that consist of RNA), thus reducing the likelihood of unwanted immunogenic reactions, as foreign proteins are not introduced into a cell or subject.

In some embodiments, the present disclosure provides methods and engineered genetic circuits for specific detection of cancer cells and production of immunomodulators (e.g., cytokines). In some embodiments, the methods and genetic circuits as provided herein are used for “bystander killing” of cancer cells, whereby memory T cells are triggered to destroy cancer cells that are not directly transformed by engineered genetic circuits of the present disclosure.

In some embodiments, the present disclosure provides methods and engineered genetic circuits for targeted expression of combinatorial immunomodulators released from specific cells (e.g., cancer cells). In some embodiments, the engineered genetic circuits encode molecules that bind to CD3, which when expressed at the surface of targeted cancer cells (anti-CD3 cells), function as synthetic T cell engagers (STEs) to directly recruit T cells to kill the cancers cells targeted/detected by the engineered genetic circuits, resulting in localized and targeted immunotherapy. In other embodiments, the engineered genetic circuits encode bi-directional T cell engagers (BiTEs), which when expressed by a cell and bound to the cell through an antigen-specific region, recruit T cells to kill the cells. BiTEs may be expressed selectively within specific cell types using engineered genetic circuits (logic gates) that provide for localized production and the same advantages observed with the use of STEs.

In some embodiments, STEs may be used as a general targeted immunotherapy, as BiTEs typically require the recognition of a tumor-specific surface antigen to trigger T cell killing.

The targeted immunotherapies of the present disclosure differ from existing therapies in that they enable systemic delivery with high efficacy and safety. In some embodiments, combination therapies using other cytokines and immunotherapy agents further enhance the efficacy of the target immunotherapy of the present disclosure.

In some embodiments, the present disclosure methods and engineered genetic circuits for the detection of aberrant cell states in diseases (including, but not limited to, autoimmune and neurological diseases) and/or for expression or secretion of immunomodulatory molecules and therapeutic molecules to modulate disease.

In some embodiments, the immunotherapy platform of the present disclosure also includes outputs (e.g., engineered genetic circuits encoding detectable molecules), which may serve as diagnostics.

Some embodiments, provide engineered nucleic acids comprising a cancer-specific promoter operably linked to a nucleic acid encoding a microRNA within an mRNA encoding an immunomodulatory molecule (e.g., a “surface T cell engager,” or STE) or a bispecific monoclonal antibody linked to microRNA binding sites.

In some embodiments, the immunomodulatory molecule or bispecific monoclonal antibody is translated only when transcription of the engineered nucleic acid is activated.

Also provided herein are engineered nucleic acid comprising a cancer-specific promoter operably linked to a nucleic acid encoding an mRNA transcript containing microRNA binding sites.

Further provided herein are engineered nucleic acids as depicted in any of FIGS. 3A-3D, 4A, 6A, 7A-7H, 9A, 14A, 15A, 16A and 17A.

The present disclosure also provides vectors comprising any of the engineered nucleic acid, as described herein. The present disclosure also provides cells comprising any of the vectors and/or engineered nucleic acid, as described herein.

Some embodiments of the present disclosure provide an engineered genetic circuit, comprising (a) a nucleic acid comprising a promoter operably linked to (i) a nucleotide sequence encoding an output messenger RNA (mRNA) containing an intronic microRNA (miRNA) and (ii) a nucleotide sequence encoding a miRNA binding site complementary to the miRNA of (a)(i), and (b) a nucleic acid comprising a promoter operably linked to a nucleotide sequence encoding at least one miRNA binding site complementary to the miRNA of (a)(i).

Other embodiments of the present disclosure provide an engineered genetic circuit, comprising (a) a first nucleic acid comprising a promoter operably linked to (i) a nucleotide sequence encoding an output messenger RNA (mRNA) containing an intronic microRNA (miRNA), (ii) a nucleotide sequence encoding an intronic miRNA, and (iii) a nucleotide sequence encoding a miRNA binding site (miRNA-BS); (b) a second nucleic acid comprising a promoter operably linked to (i) a nucleotide sequence encoding an output mRNA containing an intronic miRNA, (ii) a nucleotide sequence encoding an intronic miRNA, and (iii) a nucleotide sequence encoding a miRNA-BS; and (c) a third nucleic acid comprising a promoter operably linked to a nucleotide sequence encoding an output protein linked to a miRNA-BS, wherein the miRNA-BS of (a)(iii) is complementary to the miRNA of (b)(i), the miRNA-BS of (b)(iii) is complementary to the miRNA of (a)(i), and the miRNA-BS of (c) is complementary to the miRNA of (a)(ii) and the miRNA of (b)(ii).

Yet other embodiments of the present disclosure provide an engineered genetic circuit, comprising (a) a first nucleic acid comprising a promoter operably linked to (i) a nucleotide sequence encoding a nascent RNA transcript (e.g., a non-coding RNA transcript) containing an intronic microRNA (miRNA), and (ii) a nucleotide sequence encoding at least one miRNA binding site (miRNA-BS); (b) a second nucleic acid comprising a promoter operably linked to (i) a nucleotide sequence encoding a nascent RNA transcript containing an intronic miRNA, and (ii) a nucleotide sequence encoding at least one miRNA-BS; and (c) a third nucleic acid comprising a promoter operable linked to a nucleotide sequence encoding an output protein linked to (i) a first miRNA-BS and (ii) a second miRNA-BS, wherein the at least one miRNA-BS of (a)(ii) is complementary to the miRNA of (b)(i), the at least one miRNA-BS of (b)(iii) is complementary to the miRNA of (a)(i), the first miRNA-BS of (c)(i) is complementary to the miRNA of (a)(i), and the second miRNA-BS of (c)(ii) is complementary to the miRNA of (b)(i).

Still other embodiments of the present disclosure provide an engineered genetic circuit, comprising (a) a first nucleic acid comprising a promoter operably linked to a nucleotide sequence encoding a nascent RNA transcript containing an intronic microRNA (miRNA); (b) a second nucleic acid comprising a promoter operably linked to a nucleotide sequence encoding a nascent RNA transcript containing an intronic miRNA; and (c) a third nucleic acid comprising a promoter operable linked to a nucleotide sequence encoding an output protein linked to (i) a first miRNA-BS and (ii) a second miRNA-BS, wherein the first miRNA-BS of (c)(i) is complementary to the miRNA of (a), and the second miRNA-BS of (c)(ii) is complementary to the miRNA of (b).

Further embodiments of the present disclosure provide an engineered genetic circuit, comprising (a) a first nucleic acid comprising a promoter operably linked to a nucleotide sequence encoding a nascent RNA transcript containing an intronic microRNA (miRNA);

and (b) a second nucleic acid comprising a promoter operably linked to a nucleotide sequence encoding an output protein linked to a miRNA binding site (miRNA-BS), wherein the miRNA-BS of (b) is complementary to the miRNA of (a).

Other embodiments of the present disclosure provide an engineered genetic circuit, comprising (a) a first nucleic acid comprising a promoter operably linked to (i) a nucleotide sequence encoding an output messenger RNA (mRNA) containing an intronic microRNA (miRNA) and (ii) at least one miRNA binding site (miRNA-BS); and (b) a second nucleic acid comprising a promoter operably linked to (i) a nucleotide sequence encoding an output mRNA containing an intronic miRNA and (ii) at least one miRNA-BS, wherein the at least one miRNA-BS of (a) is complementary to the miRNA of (b), the at least one miRNA-BS of (b) is complementary to the miRNA of (a).

Still other embodiments of the present disclosure provide an engineered genetic circuit, comprising (a) a first nucleic acid comprising a promoter operably linked to a nucleotide sequence encoding a nascent RNA transcript containing an intronic microRNA (miRNA); (b) a second nucleic acid comprising a promoter operably linked to a nucleotide sequence encoding an output protein; and (c) a third nucleic acid comprising a promoter operable linked to a nucleotide sequence encoding an output protein linked to an miRNA binding site, wherein the miRNA-BS of (c) is complementary to the miRNA of (a).

Yet other embodiments of the present disclosure provide an engineered genetic circuit, comprising (a) a first nucleic acid comprising a promoter operably linked to a nucleotide sequence encoding an output protein linked to a microRNA binding site (miRNA-BS); and (b) a second nucleic acid comprising a promoter operably linked to a nucleotide sequence encoding a nascent RNA transcript containing an intronic miRNA, wherein the miRNA-BS of (a) is complementary to the miRNA of (b).

In some embodiments, the output mRNA encodes a synthetic T cell engager (STE) or a bispecific T cell engager (BiTE).

In some embodiments, the output mRNA encodes an output protein that binds to a T cell surface marker.

In some embodiments, the T cell surface marker is CD3, CD4, CD8 or CD45.

In some embodiments, the output protein is an antibody or antibody fragment that binds specifically to the T cell surface antigen.

In some embodiments, the output mRNA encodes an anti-cancer agent. For example, the output mRNA may encode a chemokine, a cytokine or a checkpoint inhibitor.

In some embodiments, a promoter is an inducible promoter. For example, a promoter may be a tumor-specific promoter (e.g., benign tumor-specific promoter or a malignant tumor-specific promoter) or a cancer-promoter.

In some embodiments, a promoter is SSX1 or H2A1.

In some embodiments, a nucleotide sequence encodes 2-5 or 2-10 micro RNA binding.

In some embodiments, an output protein is a transcription factor.

In some embodiments, an output protein is an anti-cancer agent.

In some embodiments, the output mRNA encodes a transcription factor that can bind to and activate transcription of the promoter of the at least one nucleic acid.

In some embodiments, an engineered genetic circuit comprises nucleic acids that encode a split protein system in which each protein of a functional protein dimer is encoded on a separate nucleic acid and regulated by a separate promoter.

The invention is not limited in its application to the details of construction and the arrangement of components set forth in the following description or illustrated in the drawings. The invention is capable of other embodiments and of being practiced or of being carried out in various ways. Each of the above embodiments and aspects may be linked to any other embodiment or aspect. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

The accompanying drawings are not intended to be drawn to scale. For purposes of clarity, not every component may be labeled in every drawing.

FIGS. 1A-1C. Examples of prior immunotherapy approaches. (FIG. 1A) Mode of action of chimeric antigen receptor (CAR) T cell therapy. (FIGS. 1B and 1C) Mode of action of bispecific T cell engagers.

FIGS. 2A-2B. Overview of STRICT therapy. (FIG. 2A) Using STRICT to secrete BiTE. (1) Tumor-identifying circuits are introduced into tumors by local injection or systemic administration. (2) Tumor cells transduced with the circuits secrete BiTEs, which diffuse locally, and other immunomodulatory molecules. (3) BiTEs simultaneously engage HER2 on tumor cells and T-cell receptors on local tumor-infiltrating T cells, thus triggering T cells to directly kill tumor cells. BiTEs can also recruit nearby circulating T cells to traffic to the tumor site. (4) Tumor antigens released by the first wave of killing prime and recruit more tumor-reactive T cells into play. (5) Newly recruited polyclonal T cells can kill more cancer cells, including HER2-negative tumor cells and other heterogeneous tumor cells not killed by the first wave of the anti-tumor immune response. (FIG. 2B) Using STRICT to display surface T cell engager (STE). (1) Tumor-identifying gene circuits are introduced into tumors by local injection or systemic administration. (2) Tumor cells transduced with the circuits express STEs and other immunomodulatory molecules. (3) STEs engage T-cell receptors on local tumor-infiltrating T cells, thus triggering T cells to directly kill tumor cells. (4) Tumor antigens released by the first wave of killing prime and recruit more tumor-reactive T cells into play. (5) Newly recruited polyclonal T cells can kill more cancer cells, including other heterogeneous tumor cells, and metastases, not killed by the first-wave anti-tumor immune response. Immune memory can prevent tumor relapses.

FIGS. 3A-3H. The design of RNA-only single-output AND gate. (FIGS. 3A-3D) The computation layers of all 4 input states and their and respective output states are shown. The RNA-based logic AND gate integrates the activity of two input promoters, P1 and P2, and generates an output only when both promoters are decidedly active. In this architecture, the output is the Surface T-cells Engager (STE). Promoter P1 is regulating the expression of an STE mRNA which comprises a synthetic miRNA intron (mirFF4). A negative autoregulatory feedback loop was incorporated into the circuit by encoding perfect-match mirFF4 binding sites at the 3′ end of the STE/mirFF4 transcript (mirFF4-BS). Consequently, when only promoter P1 is active the STE mRNA is constantly degraded by the cellular miRNA machinery and no STE protein is produced (State 3). Promoter P2 is regulating the expression of a miRNA sponge that includes a non-coding RNA (Decoy) with multiple bulged mirFF4 binding sites at the 3′ end. Therefore, when only promoter P2 is active, no protein output is produced (State 2). When both promoters P1 and P2 are active, the mirFF4 that is produced by the STE/mirFF4 mRNA regulated by promoter P1 is shunted away by mirFF4 sponge regulated by promoter P2, therefore allowing the production of the STE protein (State 1). (FIGS. 3E-3H) The 4 input states and their respective outputs states of the AND gate circuit when using a fluorescent protein mKate2 as the output.

FIGS. 4A-4B. mKate2 AND gate experiment results. (FIG. 4A) To examine the RNA-based logic AND gate design, it was encoded with mKate2 output. As promoter inputs for this design two human promoters we used, which are over-expressed in many human cancers: SSX1 and H2A1 (Input 1 and Input 2 respectively, whereas Input 1 encodes the mKate2 output and mirFF4). (FIG. 4B) The mKate2 output levels were measured for different designs, with respect to (a) the number of perfect-match FF4-BS encoded in input 1 and (b) two different architectures of sponge design in Input 2. X-axis annotations: M# represents Input 1 with # of FF4-BS encoded downstream to mKate2/mirFF4. For example, M3 represents Input 1 with 3 perfect-match FF4-BS, as shown in the gate illustration. S0, S1 and S2 represent three different sponge designs. S0 is a negative control transcript with no mirFF4-BS. Design S1 is Decoy transcript with 10 bulged FF4-BS encoded on the 3′, as shown in the gate illustration. Design S2 is similar to S1, but with an additional circular intron with 10 bulged FF4-BS located upstream to the 10 bulged FF4-BS which are encoded in the transcript 3′. Therefore, the gate illustration represents design M3-S1 (surrounded with green dashed lines in the plot). Results are represented in mean mKate2 expression (P1), which is the average mKate2 for cells gated for SSC/FSC in FACS to remove cell clumps and debris. Error bars represent SEM. We did not test the Input 2 condition since it does not encode the output protein anyway. NT represents non-transfected cells.

FIG. 5. mKate2 AND gate experiment results. To again examine the RNA-based logic AND gate design, it was encoded with mKate2 output. ECFP was encoded in the sponge transcript to measure the degradation of sponge by the miRNA. SSX1 and H2A1 were used promoter inputs for this design: Input 1 and Input 2 respectively, whereas Input 1 encodes the mKate2 output and mirFF4. The mKate2 and ECFP output level for different experimental settings were measured, with respect to (a) the number of perfect-match FF4-BS encoded in input 1 and (b) two different architectures of sponge design in Input 2. X-axis annotations: M# represents Input 1 with # of FF4-BS encoded downstream to mKate2/mirFF4.

FIGS. 6A-6B. The design of multi-output AND-gate circuit. (FIG. 6A) When both promoters P1 and P2 are active, the mirFF4 that is produced by the TF/mirFF4 mRNA regulated by promoter P1 is shunted away by mirFF4 sponge regulated by promoter P2, therefore allowing the production of an artificial transcription factor (TF). The TF will further bind to its promoter and trigger the transcription of multiple user-defined outputs. (FIG. 6B) The output level of multi-output AND-gate is tunable. CXCL10 is CXCL1p regulating a GAL4BD-VP16AD harboring a mirFF4v2B intron and 10 downstream mirFF4-Bs. SSX10 is SSX1p regulating a GAL4BD-VP16AD harboring a mirFF4v2B intron and 10 downstream mirFF4-Bs. SSX*10 is truncated SSX1p in which part of the 5′ UTR was removed together with the KOZAK sequence, regulating a GAL4BD-VP16AD harboring a mirFF4v2B intron and 10 downstream mirFF4-Bs. Sponge S0 is a negative control transcript WO mirFF4-BS. Sponge S2 is Decoy transcript with 10 bulged FF4-BS encoded on the 3, with an additional circular intron with 10 bulged mirFF4-BS located upstream to the 10 bulged mirFF4-BS which are encoded in the transcript 3′. In all samples, the mKate2 output is encoded in under a G5p (a promoter containing 5 GALA binding sites). The output levels are tunable by using different strength of promoters as P1 and different architecture of sponges.

FIGS. 7A-7H. The design of several Boolean logic gates. Schematic illustration of RNA-based designs for AND, NAND, XNOR, NOR, NOT, XOR, IMPLY, NIMPLY gate. OP: Output protein; Nan: nascent RNA transcript.

FIG. 8. Anti-HER2 bispecific T cell engager (BiTE) and surface T cell engager (STE) trigger T cells to mediate robust tumor killing and IFN-γ secretion. HEK-293T (minimally expressing HER2) cells were transfected with various DNA constructs as indicated. 48 hrs post transfection, various HEK-293T cells were harvested and co-cultured with human T cells for 5 hrs or 24 hrs. 5 hr cytotoxicity by T cells was measured by LDH release assay and 24 hr IFN-γ secretion by T cells was measured by IFN-γ ELISA. Data show that T cells mediate robust tumor killing and IFN-γ secretion on BiTE secreting tumor cells (group 1-2). The tumor killing and IFN-γ secretion correlate with HER2 expression level on tumor cells (group 1-2). T cells also mediate robust tumor killing and IFN-γ secretion on STE expressing tumor cells (group 3-6), and the cytotoxicity and IFN-γ secretion are independent of tumor antigen (HER2) expression (group 3-6). Furthermore, T cells mediate minimal tumor killing and IFN-γ secretion when co-cultured with HEK-293T cells expressing non-BiTE and non-STE control proteins (group 7-9).

FIGS. 9A-9C. Single-output AND gate architecture can be harnessed to fine tune T cell killing efficiency of tumor cells. HEK-293T cells were transfected with various DNA constructs as indicated. (FIG. 9A) Design of single-output AND gate driving STE expression. (FIG. 9B) Experiment result of mKate AND gate. (1,0) indicated cells transfected with P1 module only. (1,1) indicated cells transfected with P1 and P2 modules. (0,0) represents non-transfected cells. (FIG. 9C) Experiment result of STE AND gate. (1,0) indicated cells transfected with P1 module only. (1,1) indicated cells transfected with P1 and P2 modules. (0,0) indicated cells transfected with a non-STE protein. Ctrl indicated non-transfected cells. 48 hrs post transfection, various HEK-293T cells were harvested and co-cultured with human T cells for 5 hrs. Cytotoxicity by T cells was measured by LDH release assay. Data show that T cells kill 293T transfected with P1 module (column 1) and the killing can be greatly enhanced by the AND gate architecture (column 2). T cells exhibit minimal killing on non-STE expressing cells (column 3 & 4).

FIG. 10. Anti-HER2 bispecific T cell engager (BiTE) and surface T cell engager (STE) trigger T cells to mediate robust tumor killing and IFN-γ secretion. Stable 4T1 cells (HER2-) expressing indicated DNA constructs were co-cultured with human T cells for 5 hrs or 24 hrs. 5 hr cytotoxicity by T cells was measured by LDH release assay and 24 hr IFN-γ secretion by T cells was measured by IFN-γ ELISA. Data show that T cells mediate minimal killing and IFN-γ secretion on HER2- or STE-tumor cells. (group 1 & 3). T cells mediate robust tumor killing and IFN-γ secretion on STE-expressing tumor cells. (group 2). T cells also mediate robust tumor killing and IFN-γ secretion when co-cultured with cell mixtures consisting of low numbers of BiTE secreting cells with non-BiTE secreting tumor. This indicates minimal numbers of BiTE secreting cells in the tumor mass can elicit robust tumor mass killing and IFN-γ release (group 4).

FIG. 11. anti-HER2 bispecific T cell engager (BiTE) and surface T cell engager (STE) trigger T cells to mediate robust tumor killing on human breast cancer cell line. Stable MDA-MB453 (HER2+) cell lines were created by lentiviral transduction with various DNA constructs as indicated. Various MDA-MB453 cells were harvested and co-cultured with human T cells for 5 hrs. 5 hr cytotoxicity by T cells was measured by LDH release assay. Data show that T cells mediate robust tumor killing on BiTE secreting tumor cells (group 2). T cells also mediate robust tumor killing on STE expressing tumor cells (group 3-4). Furthermore, T cells mediate minimal tumor killing when co-cultured with parental MDA-MB453 tumor cell line (group 1).

FIG. 12. The design of 2 versions of STE. For version 1 (v1), anti-CD3ε scFv is fused with an inert transmembrane protein (DARC). For version 2 (v2), anti-CD3ε scFv is fused with human IgG1-Hinge-CH2-CH3 domain, followed by murine B7.1-transmembrane (TM) and cytoplasmic (CYP) domains.

FIG. 13. Surface T cell engager (STE) version 1 (v1) and version 2 (v2) both trigger T cells to mediate robust tumor killing on HEK-293T cells. Various inducible STE expressing HEK-293T cell lines were created by lentiviral transduction. Various HEK-293T cells were harvested and co-cultured with human T cells for 5 hrs. 5 hr cytotoxicity by T cells was measured by LDH release assay. Data show that T cells mediate robust tumor killing on transfected STEv1 expressing tumor cells (column 2). T cells also mediate robust tumor killing on inducible STEv1 and STEv2 expressing tumor cells (column 3 and 4). Furthermore, T cells mediate minimal tumor killing when co-cultured with non-STE expressing HEK-293T cell line (column 1).

FIG. 14. AND gate architecture can be harnessed to fine tune T cell killing efficiency of tumor cells. (A) The design of multi-output AND gate for STE expression. (B) HEK-293T cells were transfected with various DNA constructs as indicated. (1,0) indicated cells transfected with STE only. (1,1) indicated cells transfected with STE and sponge. (0,0) indicated cells transfected with a non-STE protein. 48 hrs post transfection, various HEK-293T cells were harvested and co-cultured with human T cells for 5 hrs. Cytotoxicity by T cells was measured by LDH release assay. Data show that T cells kill STE expressing (1,0) cells (column 2 and 4) and the killing can be greatly enhanced by the AND gate (1,1) architecture (column 3 and 5). T cells exhibit minimal killing on non-STE expressing cells (column 1).

FIG. 15. AND gate architecture can be harnessed to fine tune T cell killing efficiency of tumor cells. (A) The design of multi-output AND gate for STE expression. (B) HEK-293T cells were transfected with various DNA constructs as indicated. (1,0) indicated cells transfected with STE only. (1,1) indicated cells transfected with STE and sponge. (0,0) indicated cells transfected with a non-STE protein. 48 hrs post transfection, various HEK-293T cells were harvested and co-cultured with human T cells for 5 hrs. Cytotoxicity by T cells was measured by LDH release assay. Data show that T cells kill STE expressing (1,0) cells (column 3 and 5) and the killing can be greatly enhanced by the AND gate (1,1) architecture (column 4 and 6). T cells exhibit minimal killing on non-STE expressing cells (column 1). The killing on (1,0) condition is mainly caused by the leakage of GALA promoter output (column 2 v. 3 or 5). Further modification may be made to decrease the leakage of GALA promoter output (STE v1). We will decrease the GALA promoter leakage by removing the KOZAK sequence of STE v1, making STE v1 output self-degrading by adding miRNA binding sites at 3′ end, and the combination of both mechanisms.

FIG. 16. GALA-gate v2 architecture can be harnessed to fine tune T cell killing efficiency of tumor cells and exhibit less cytotoxicity at (1,0) state. (A) The design of multi-output AND gate for STE expression. (B) HEK-293T cells were transfected with various DNA constructs as indicated. (1,0) indicated cells transfected with STE only. (1,1) indicated cells transfected with STE and sponge. (0,0) indicated cells transfected with a non-STE protein. 48 hrs post transfection, various HEK-293T cells were harvested and co-cultured with human T cells for 5 hrs. Cytotoxicity by T cells was measured by LDH release assay. Data show that T cells kill STE expressing (1,0) cells (column 3) and the killing can be enhanced by the AND gate (1,1) architecture (column 4). T cells exhibit minimal killing on not STE expressing cells (column 1). The killing on (1,0) state of this version is improved compared to GALA gate v1 architecture (v2 is more closer to basal level (0,0)). Further modification may be made to decrease the killing at (1,0) state. We will decrease the GALA promoter output at (1,0) state by adding miR binding sites at 3′ end of STE gene.

FIG. 17. GALA-gate v3 architecture can be harnessed to fine tune T cell killing efficiency of tumor cells and exhibit less cytotoxicity at (1,0) state. (A) The design of multi-output AND gate for STE expression. (B) HEK-293T cells were transfected with various DNA constructs as indicated. (1,0) indicated cells transfected with STE only. (1,1) indicated cells transfected with STE and sponge. (0,0) indicated cells transfected with a non-STE protein. 48 hrs post transfection, various HEK-293T cells were harvested and co-cultured with human T cells for 5 hrs. Cytotoxicity by T cells was measured by LDH release assay. Data show that T cells minimally kill STE expressing (1,0) cells (column 3) and only reach efficient killing when the AND gate is active (1,1) (column 4). T cells exhibit minimal killing on not STE expressing cells (column 1). The killing on (1,0) state is as low as (0,0) state. Further modification, such as increasing GAL4-VP16 output level or increasing GALA binding sites, can be done to enhance the killing efficacy of (1,1) state.

FIG. 18. Overview of Synthetic Tumor-Recruited Immuno-Cellular Therapy (STRICT). Panel 1: Tumor-targeting gene circuits, are designed to integrate the activity of two tumor-specific synthetic promoters and generate the expression of synthetic and natural immunomodulators only when both promoters are active, which provides high tumor-selectivity to our circuit; Panel 2: The circuit is delivered in vivo using hydrogel-based delivery; Panel 3: Only transduced cancer cells express synthetic Surface T-cells Engager (STE) and/or native immunomodulators that recruit T-cells to kill tumor cells; Panel 4: tumor cells are eliminated by activated T-cells.

FIG. 19. The design of RNA-only single-output AND gate. The RNA-based logic AND gate integrates the activity of two input promoters, P1 and P2, and generates and output only when both promoters are decidedly active. In this architecture, the output is a fluorescent protein mKate2. Promoter P1 is regulating the expression of an mKate2 mRNA which comprises a synthetic miRNA intron (miR1). We incorporated a negative autoregulatory feedback loop into the circuit by encoding perfect-match miR1 binding sites at the 3′ end of the mKate2/miR1 transcript (miR1-BS). Consequently, only when both promoters P1 and P2 are active, the mirR1 that is produced by the mKate2/miR1 mRNA regulated by promoter P1 is shunted out by the miR1 sponge regulated by promoter P2, therefore allowing the production of the mKate2 protein.

FIG. 20. RNA-only single-output AND gate design. The top panel depicts the design details of RNA-only single-output AND gate. The left table shows that miRNA binding sequences affect the sponging activity. The right panel shows that mKate2 fold-induction by each sponge and the ECFP level reduction by miR1.

FIG. 21. The number of binding sites in the sponge and the abundance of sponge transcripts affect the sponging activity. Left panel shows the design details of module 1 (M1) and various sponges (S67, S73, and S62). Right upper panel shows the raw output level of mKate2 and ECFP of various experimental conditions. Right lower panel shows the mKate2 fold induction by each sponge. SC represents control sponge (no binding sites).

FIG. 22. Sponge architectures affect the sponging activity. Left panel shows the design details of various sponges (S76, S99, S100, and S101). Right upper panel shows the raw output level of mKate2 and ECFP of various experimental conditions. Right lower panel shows the mKate2 fold induction by each sponge. SC represents control sponge (no binding sites).

FIG. 23. miRNA backbone affects gate performance. Left panel shows the design details of module 1 (M) and various sponges (Sx and S76). Right upper panel shows the raw output level of mKate2 and ECFP of various experimental conditions. Right lower panel shows the mKate2 fold induction of various module 1 constructs (M1, M2A, and M2B are 3 versions of module 1, each consisting of a different miRNA backbone) by various sponges. SC represents control sponge (no binding sites).

FIGS. 24A-24B. Doxycycline inducible STE can trigger T cells to efficiently kill OVCAR8 ovarian cancer cells, HEK-293T cells and secrete IFN-g. 3 versions of Dox-inducible STE (STE, STEv2, and STE-snap) all can trigger robust cellular killing and IFN-g secretion by T cell.

FIG. 25. Multiple-output circuit stringently kills tumor cells. (FIG. 25A) GAD outputted by the AND gate can target a third promoter (P3), which can express multiple proteins, such as STE and immunomodulatory molecules. (FIG. 25B) HEK-293T cells transfected with gene circuits encoding: HEK/DARC (0,0)—a non-STE protein; GAD gate (1,0)—the P1+P3 constructs only, where P3 expresses an STE; GAD gate (1,1)—the P1+P2+P3 constructs, where P3 expresses an STE; HEK/const—constitutively expressed STE. 48 h post-transfection, cells were co-cultured with human T cells for 5 hrs. Cytotoxicity was measured by LDH release assay. T cells killed efficiently only when AND gate is ON (1,1). T cells minimally kill STE-negative cells (0,0). Killing in the (1,0) state is as low as on (0,0) state. Increasing GAD expression the number GAD-binding sites may further enhance the efficacy of the (1,1) state.

FIG. 26. Synthetic tumor-specific promoters exhibit higher tumor specificity than native ones. (A) The top panel illustrates the design of synthetic tumor-specific promoters. 16 transcription factor binding sites were cloned in tandem upstream of a minimal promoter (late adenovirus promoter). The lower panel shows that synthetic tumor-specific promoters exhibit higher tumor specificity than native ones. H2A1p is a native tumor-specific promoters. S9 to S19 are selective examples of synthetic promoters and the parentheses denote their transcription factor binding sites. OVCAR8: ovarian cancer cells. IOSE120, IOSE386: immortalized normal ovarian epithelial cells. aHDF: adult human dermal fibroblast. CCD: normal colon fibroblast. MCF10A, MCF12A: immortalized normal breast cells. (B) The top panel illustrates the design of synthetic tumor-specific promoters. 16 transcription factor binding sites were cloned in tandem upstream of a minimal promoter (late adenovirus promoter). The lower panel shows that synthetic tumor-specific promoters exhibit higher tumor specificity than native ones. SSX1 and H2A1p are native tumor-specific promoters. S9 to S28 are selective examples of synthetic promoters and the parentheses denote their transcription factor binding sites. aHDF: adult human dermal fibroblast. HOV-epi: primary ovarian epithelial cells. OVCAR8: ovarian cancer cells.

FIG. 27. Multi-output AND gate exhibits significantly higher output level in tumor cells than in normal cells. The circuit depicted at the top panel exhibits around 90-fold higher activity in tumor cells (OVCAR8) than in normal cells (ISOE120).

FIG. 28. Multi-output AND gate exhibits significantly higher output level in tumor cells than in normal cells. When both promoters are active, G8-F circuit exhibits around 90-fold higher activity in tumor cells (OVCAR8) than in normal cells (ISOE120). The output level of G8-F gate is also higher than the input promoter activity level.

FIG. 29. The output level of circuit on tumor cells can be tuned by modifying the number of GAD binding sites in the GAD promoter and adjusting the number of miRNA binding sites on the downstream output transcripts. The output of G8-F gate is also higher than the input promoter (S19p) activity.

FIG. 30. Multi-output AND gate exhibits significantly higher output level in tumor cells than in normal cells. When both promoters are active, G8-F circuit exhibits around 90-fold higher activity in tumor cells (OVCAR8) than in normal cells (ISOE120). The output of G8-F gate is also higher than the input promoter activity.

FIG. 31. Multi-output circuit specifically triggers T cells to kill tumors cells and secrete IFN-g. (A) STE triggers robust T-cell killing of circuit-transduced tumor cells (OVCAR8) but not normal cells (aHDF, HOV-epi). Circuit also triggers minimal tumor killing at state (1,0). (B) STE triggers robust T-cell killing of circuit-transduced tumor cells (OVCAR8) but not normal cells (aHDF, HOV-epi). Circuit also triggers minimal tumor killing at state (1,0). (C) T cells mediated strong IFN-g secretion by circuit-transduced tumor cells but not normal cells.

FIG. 32. Different multi-output circuits exhibit different levels of anti-tumor specificity. G8-Fv1 and G14-Fv1 triggers significantly higher tumor cell (OVCAR8) killing than normal cell (IOSE386) killing. G8 (a promoter containing 8 GALA binding sites), G14 (a promoter containing 14 GAL4 binding sites)

FIG. 33. Different multi-output circuits exhibit different levels of anti-tumor specificity. (FIG. 33A) Several gate designs (G5-Fv1, G8-Fv1, G14-Fv1, G5-Fv2, G8-Fv2, G14-Fv2) can trigger significantly higher IFN-g secretion by T cells on tumor cells (OVCAR8) than normal cells (IOSE386). (FIG. 33B) G8-F gate triggers T cells to secrete copious amount of IFN-g on tumor cells (OVCAR8) but not normal cells (aHDF, HOV-epi).

FIG. 34. STEs potently decrease pancreatic tumor burden in vivo. NB508 tumor cells displaying doxycycline (Dox)-inducible STEs were injected subcutaneously. 10 days post-inoculation, mice were randomized into Dox-induced or untreated arms. Top panel: Significant growth reduction was observed in Dox-induced tumors (+Dox) vs. untreated controls (nt). Two +Dox mice in were sacrificed prematurely at day 17 due to skin irritation. Lower panel: Whole tumors dissected at day 21 post-treatment are significantly smaller.

FIG. 35. Combination immunotherapies triggered by STRICT reduced tumor burden significantly in an intraperitoneally-disseminated ovarian cancer model. (A) The experimental plan and treatment schedule. (B) Combination therapy triggered by STRICT significantly reduced tumor burden. The left panel represents the tumor burden of control groups. The right panel represents the tumor burden of treated groups. The parentheses denote the combination therapy strategy.

FIG. 36. Combination immunotherapies triggered by STRICT reduced tumor burden significantly in an intraperitoneally-disseminated ovarian cancer model. This is the same data as FIG. 35, but now all the groups are plotted in the same graph.

FIG. 37. Combination immunotherapies triggered by STRICT reduced tumor burden significantly in an intraperitoneally-disseminated ovarian cancer model. This is the same data as FIG. 35, but plotted differently.

FIG. 38. Combination immunotherapies triggered by STRICT reduced tumor burden significantly in an intraperitoneally-disseminated ovarian cancer model. This is the same data as FIG. 35, but plotted differently.

FIG. 39A. Combination immunotherapies triggered by STRICT reduced tumor burden significantly in an intraperitoneally-disseminated ovarian cancer model. This is the same data as FIG. 35, but tumor growth curves of individual mice and the average burden of each group were shown.

FIG. 40. Combination immunotherapies triggered by STRICT reduced tumor burden significantly in an intraperitoneally-disseminated ovarian cancer model. This is the same data as FIG. 35 except group S15p-GAD+G8p-STE-F were not shown, tumor burden of each imaging time point and the average burden of each group were shown. G8p (a promoter containing 8 GAL4 binding sites).

FIG. 41. Combination immunotherapies triggered by STRICT reduced tumor burden significantly in an intraperitoneally-disseminated ovarian cancer model. This is the same data as FIG. 35, but now the bioluminescent images of tumor burden of each individual mouse at day 36 post tumor inoculation were shown.

FIG. 42. Combination immunotherapies triggered by STRICT reduced tumor burden significantly in an intraperitoneally-disseminated ovarian cancer model. This is the same data as FIG. 35, but now the bioluminescent images of tumor burden of each individual mouse at day 43 post tumor inoculation were shown.

FIG. 43. Combination immunotherapies triggered by STRICT reduced tumor burden significantly in an intraperitoneally-disseminated ovarian cancer model. This is the same data as FIG. 35, but now the bioluminescent images of tumor burden of each individual mouse at day 7 and day 43 post tumor inoculation were shown.

FIG. 44. The pipeline of identifying cancer-specific synthetic promoters. A library of synthetic promoters driving mKate2 expression was introduced into normal cells and cancer cells with lentivirus. The mKate2 positive cells were sorted and next generation sequencing was utilized to identify the enriched synthetic promoter sequence for each cell type. The synthetic promoter sequences highly enriched in cancer cell but not in normal cells will be cloned and there tumor-specific activity will be further validated.

FIG. 45. The design of synthetic promoter library. Design 1 constitutive of all permutations of 8 mer sequences built in tandem (12 time repeat) without spacer in between each 8 mers. Design 2 constitutive of all permutations of 8 mer sequences built in tandem (9 time repeat) with a 3 mer spacer in between each 8 mers. Design 3 constitutive of selective 11 mer sequences built in tandem (7 time repeat) without a 3 mer spacer in between each 11 mers.

FIG. 46. The activity of selected synthetic promoters. The activity of 40 synthetic promoters isolated from FACS sorting was tested on 3 different cancer cell lines. We observed that these 40 synthetic promoters can provide us a wide range of transcription activity.

FIG. 47. The normalized activity of selected synthetic promoters. The activity of 40 synthetic promoters isolated from FACS sorting were tested on 3 different cancer cell lines. We observed that these 40 synthetic promoters can provide us a wide range of transcription activity. The data is normalized to the constitutive promoter (UbCp) for each cell line.

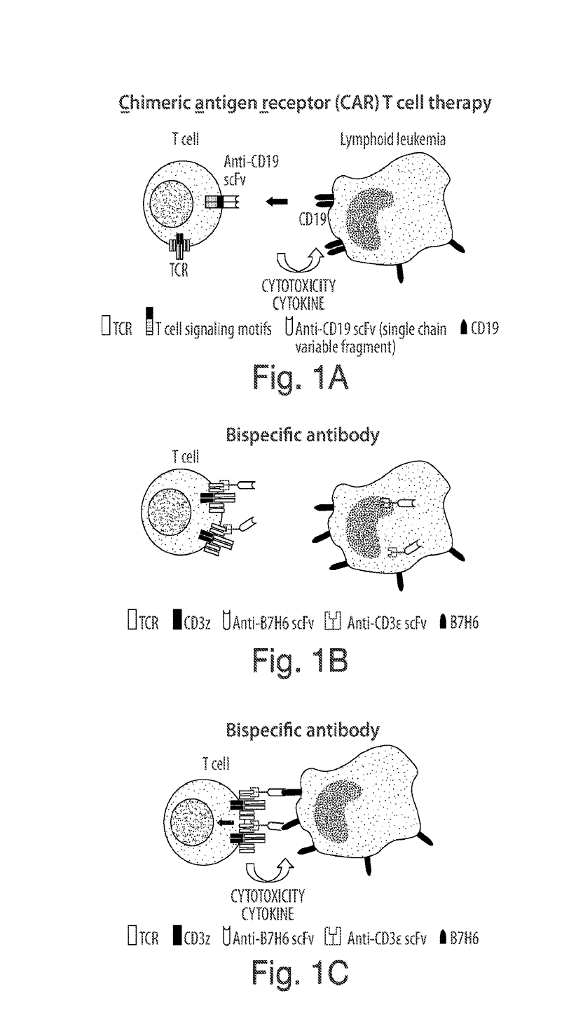

### DETAILED DESCRIPTION

Synthetic Tumor Recruited Immuno-Cellular Therapy (STRICT) of the present disclosure includes cell-specific diagnostic and therapeutic circuits (engineered genetic circuits/logic gates) having, in some embodiments, combinatorial immunomodulatory outputs (e.g., antigens and cytokines). The cell-specific genetic circuits are based primarily on RNA, thus typically do not elicit adverse immunogenic reactions in a subject. The combinatorial immunomodulatory outputs may include, for example, Synthetic T Cell Engagers (STEs), Bi-directional T Cell Engagers (BiTEs), antibodies, antibody fragments, cytokines and other molecules that elicit a cytotoxic T cell response.

In some aspects of the present disclosure, GALA gates enable tunable multi-output combinatorial therapy. Additional key immune modulators, as circuit outputs, can be implemented for effective combinatorial therapy. In some embodiments, cytokines may be used to enhance immune cell function; for example, IL-12 may be used to enhance Th1 response and to revert to a suppressive tumor microenvironment. In some embodiments, chemokines may be used to recruit immune cells; for example, CCL21 may be used to recruit CCR7+ T cell populations. In some embodiments, immune checkpoint blockade inhibitors may be used to enhance anti-cancer immunity; for example, anti-PD1 mAb, anti-PDL1 mAb, and anti-CTLA4 mAb).

Further, in some embodiments, anti-HER2 BiTE triggers T cells to mediate robust HER2+ tumor killing and cytokine production. In some embodiments, various STEs can trigger T cell killing of various types of tumor cells. In some embodiments, RNA AND gate architecture can be harnessed to fine tune STE expressing level and T cell tumor killing efficiency. In some embodiments, a low ratio of BiTE secreting cells in whole tumor population is enough to trigger robust tumor killing.

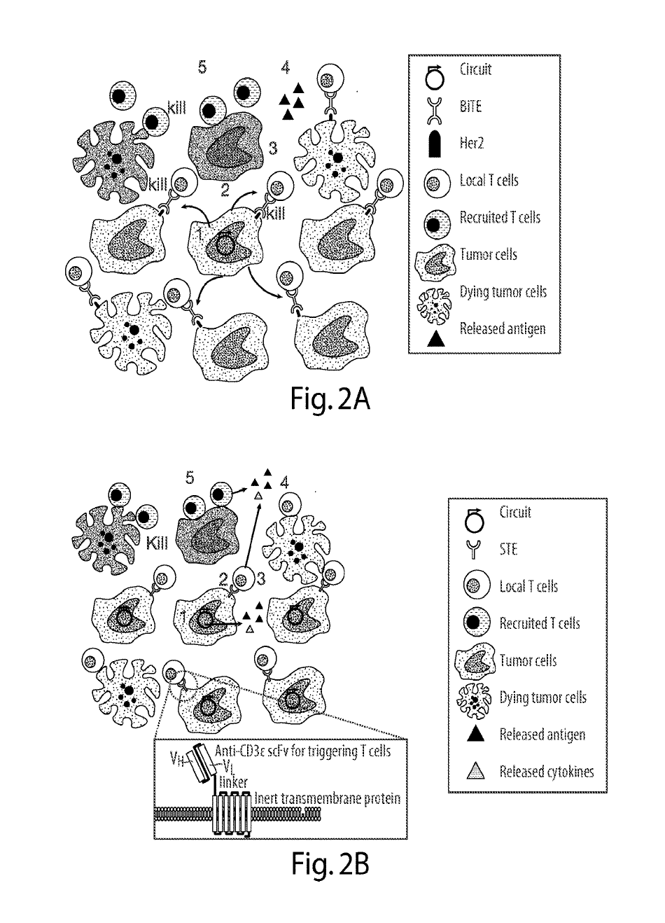

As depicted in FIGS. 2A and 2B, the methods provided herein lead to the targeted destruction of cancer cells. For example, tumor-identifying genetic circuits are first introduced into tumors by local injection or systemic administration (FIGS. 2A(1) and 2B(1)). Then, tumor cells transduced with the genetic circuits display Surface T-cell Engagers (STEs) and express immunomodulatory molecules (FIG. 2A(2)). STEs engage T-cell receptors on local tumor-infiltrating T cells and trigger the T cells to eradicate tumor cells (FIG. 2A(3)). Tumor antigens released by the first wave of eradication then primes and recruits more tumor-reactive T cells (FIG. 2A(4)). Newly recruited polyclonal T cells eradicate more cancer cells, including other heterogeneous tumor cells and metastases not eradicated by the first-wave anti-tumor immune response (FIG. 2A(5)) Immune memory prevents tumor relapses.

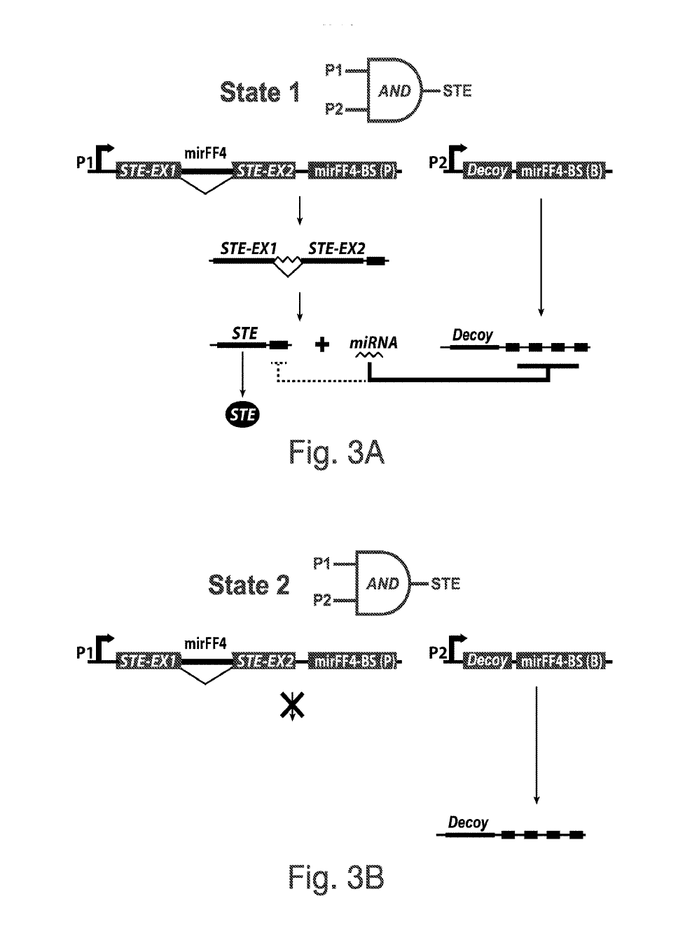

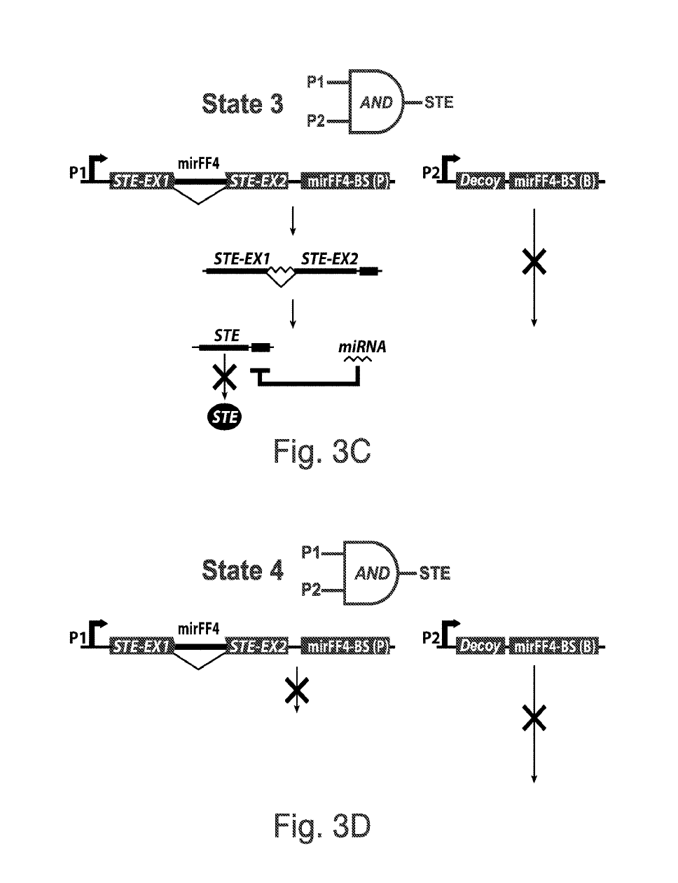

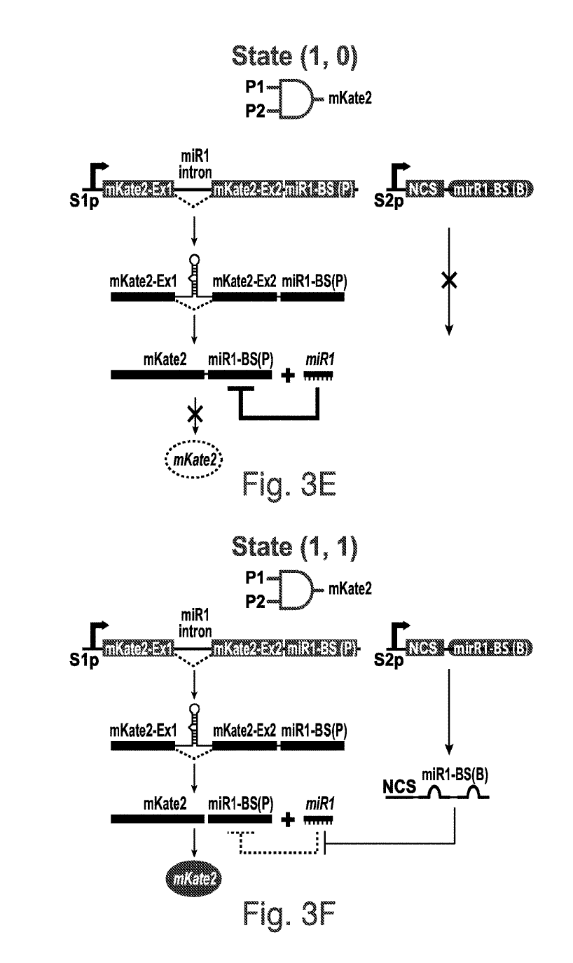

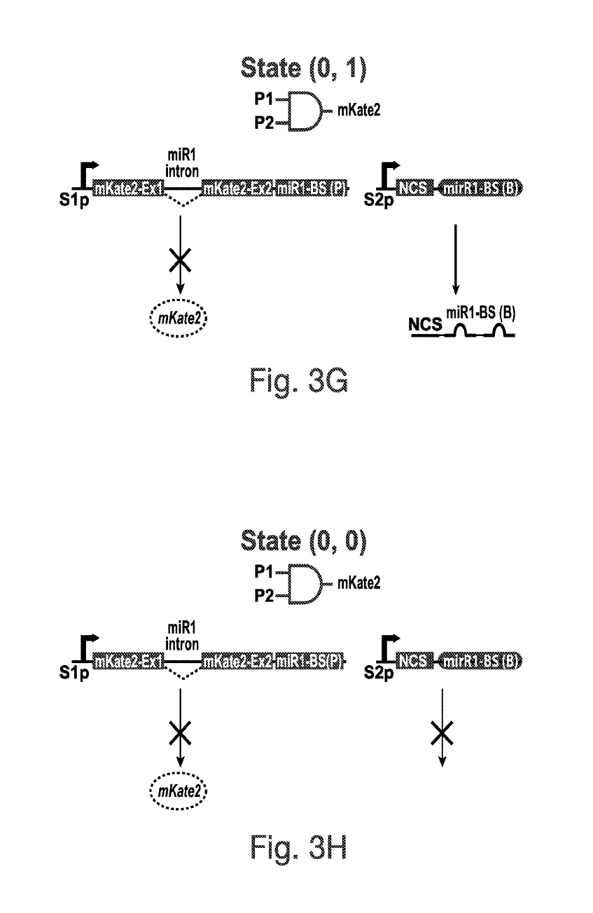

FIGS. 3A-3D depict RNA-based logic AND gates. The RNA-based logic AND gate integrates the activity of two input promoters, P1 and P2, and generates and output only when both promoters are decidedly active. In this architecture, the output is the Surface T-cell Engager (STE). Promoter P1 is regulating the expression of an STE mRNA that comprises a synthetic miRNA intron (mirFF4). A negative autoregulatory feedback loop was incorporated into the circuit by encoding perfect-match mirFF4 binding sites at the 3′ end of the STE/mirFF4 transcript (mirFF4-BS). Consequently, when only promoter P1 is active the STE mRNA is constantly degraded by the cellular miRNA machinery and no STE protein is produced (FIG. 3C, State 3). Promoter P2 regulates the expression of a miRNA sponge containing a non-coding RNA (Decoy) with multiple bulged mirFF4 binding sites at the 3′ end. Therefore, when only promoter P2 is active, no protein output is produced (FIG. 3B, State 2). When both promoters P1 and P2 are active, the mirFF4 that is produced by the STE/mirFF4 mRNA regulated by promoter P1 is tittered out by the mirFF4 sponge regulated by promoter P2, therefore allowing the production of the STE protein (FIG. 3A, State 1).

Some embodiments of the present disclosure provide engineered genetic circuits that include (a) a first nucleic acid comprising a first promoter operably linked to (i) a nucleotide sequence encoding an output messenger RNA (mRNA) containing an intronic micro RNA (miRNA) and (ii) a nucleotide sequence encoding at least one miRNA binding site complementary to the miRNA of (a)(i), and (b) a second nucleic acid comprising a second promoter different from the first promoter and operably linked to a nucleotide sequence encoding at least one miRNA binding site complementary to the miRNA of (a)(i).

In some embodiments, the output mRNA encodes an output protein that binds to a T cell surface marker. For example, an output protein may be a protein that elicits a cytotoxic T cell response. Thus, an output protein may be a receptor that binds to an antigen (e.g., a CD3 antigen) on the surface of a T cell. The surface marker may be, for example, CD3, CD4, CD 8 or CD45. Other T cell surface markers are encompassed by the present disclosure. In some embodiments, the output protein is an antibody or antibody fragment that binds specifically to the T cell surface antigen.

Specific non-limiting examples of output proteins are depict in FIG. 12. “STE v1” includes anti-CD3ε scFV V_{L} and V_{H} domains for triggering T cells. Thus, in some embodiments, the first nucleic acid of a genetic circuit comprises a first promoter operably linked to a nucleotide sequence encoding an output messenger RNA (mRNA) (containing an intronic micro RNA (miRNA)) that encodes anti-CD3ε scFV V_{L} and V_{H} domains of a transmembrane protein. “STE v2” includes anti-CD3ε scFv fused with human IgG1-Hinge-CH2-CH3 domain, followed by murine B7.1-transmembrane (TM) and cytoplasmic (CYP) domains. Thus, in some embodiments, the first nucleic acid of a genetic circuit comprises a first promoter operably linked to a nucleotide sequence encoding an output messenger RNA (mRNA) (containing an intronic micro RNA (miRNA)) that encodes anti-CD3ε scFv fused with human IgG1-Hinge-CH2-CH3 domain, followed by murine B7.1-transmembrane and cytoplasmic domains

In some embodiments, the output mRNA encodes a chemokine, a cytokine or a checkpoint inhibitor.

In some embodiments, the first promoter and/or the second promoter is an inducible promoter. Typically, the first promoter is different from the second promoter. For example, the promoters in genetic circuit, in some embodiments, may be regulated by different input signals (e.g., different transcription factors) present in a cell—Input 1 regulates the first promoter, Input 2 regulates the second promoter.

The first and/or second promoter (the first promoter, the second promoter, or both promoters) may be tumor-specific promoters (or disease-specific promoters), meaning that they are regulated by signals that are only expressed by tumor cells or cancer cells (or other disease cell) or by signals that are expressed in tumor/cancer cells at a level that is at least 30% (e.g., at least 40%, 50%, 60%, 70%, 80, 90%) higher than the level expressed in non-tumor/non-cancer cells.

Engineered nucleic acids of the genetic circuits, as provided herein, may include miRNA binding sites. A miRNA binding site is a nucleotide sequence to which a miRNA binds—a miRNA binding site is complementary the miRNA. Thus, a miRNA is said to bind to its cognate miRNA binding site. An engineered nucleic acid may contain 1-50 mirRNA binding sites. In some embodiments, an engineered nucleic acid encoding a decoy molecule (that functions to “soak up” cognate miRNA in a cell) encodes 5-10, 5-20 or 5-30 miRNA binding sites. In some embodiments, an engineered nucleic acid encoding a decoy molecule encodes 5, 6, 7, 8, 9, 10, 11, 12, 13, 14, 15, 16, 17, 18, 19 or 20 mRNA binding sites. In some embodiments, an engineered nucleic acid encoding an output mRNA, such as a STE mRNA, encodes 1-5 or 1-10 miRNA binding sites. In some embodiments, an engineered nucleic acid encoding an output mRNA encodes 1, 2, 3, 4, 5, 6, 7, 8, 9 or 10 mRNA binding sites. Typically, the number of miRNA binding sites on an mRNA encoding an immunomodulatory molecule is less than the number of miRNA binding sites on a decoy RNA (e.g., a promoter operably linked to a nucleic acid encoding miRNA binding sites and, optionally, non-coding mRNA). The length of an miRNA, and thus a cognate mRNA binding site, may vary. In some embodiments, the length of an miRNA is 15-50, 15-40, 15-30 or 15-20 nucleotides. In some embodiments, the length of an miRNA is 15, 16, 17, 18, 19, 20, 21, 22, 23, 24, 25, 26, 27, 28, 29 or 30 nucleotides.

In some embodiments, an output protein is a transcription factor (e.g., a protein that binds to DNA to control the rate of transcription).

Engineered Nucleic Acids and Genetic Circuits

The present disclosure provides engineered genetic circuits that are capable of triggering, from within a tumor/cancer cell, immunotherapy against that tumor/cancer cell and surrounding cancer cells. An “genetic circuit” refers to a collection of molecules (e.g., nucleic acids and proteins, such as transcription factors, co-factors and polymerases) that interact with each other in a cell to control expression of mRNA and proteins. Genetic circuits, as provided herein, typically include at least two nucleic acids, one encoding an output messenger RNA (mRNA) containing and intronic micro RNA (miRNA), and another encoding several miRNA binding sites. An “intronic miRNA” is a miRNA that is positioned within an mRNA transcript between two exons that together encode an output molecule. An intronic miRNA is “spliced out” of the mRNA transcript during transcript maturation. For example, with reference to FIG. 3A, ‘STE-EX1-mirFF4-STE-EX2’ (top row) represents a DNA sequence encoding micro RNA mirFF4 positioned between two exons of gene encoding a synthetic T cell engager (STE). The construct in the second row of FIG. 3 represents an mRNA transcript encoding the STE, undergoing maturation, whereby the intronic micro RNA mirFF4 is removed by RNA splicing. The mature mRNA encoding the STE may then be translated to produce the STE protein, depending on whether a decoy molecule (a molecule containing cognate mirFF4 binding sites) is present in the cell.

Thus, an “output messenger RNA” or “output mRNA” refers simply to mRNA encoded by a particular nucleotide sequence of an engineered nucleic acid. Output mRNA, typically including an intronic micro RNA, in some embodiments, encodes a output protein that binds to a T cell surface marker. In some embodiments, an output mRNA encodes an anti-cancer agent. An “anti-cancer” agent is any substance or molecule that, when exposed to a cancer cell, can be used to kill the cancer cell, or reduce the rate of cell division of the cancer cell (e.g., by at least 10%, 20%, 30%, 40% or 50% relative to the cancer cell not exposed to the anti-cancer agent). In some embodiments, an output mRNA encodes a killer gene, a neoantigen, a metabolic enzyme that degrade metabolites on which cancer cells depend for growth and/or survival, a chemokine, a cytokine or a checkpoint inhibitor, as discussed elsewhere herein. Other anti-cancer agents are encompassed by the present disclosure.

Genetic circuits of the present disclosure may also be referred to as, or function as, “logic gates,” which typically have two inputs and one output, although more or less inputs and/or outputs are encompassed by the present disclosure. Logic gates (e.g., AND, OR, XOR, NOT, NAND, NOR and XNOR) may be described in terms of an “ON” state, in which an output is produced, and an “OFF” state, in which an output is not produced. With genetic logic gates, each “input” may be regulated by an independent promoter, each promoter responsible for activating transcription of a nucleic acid encoding an output or a molecule that regulates the production of and/or the expression level of an output molecule. For example, FIGS. 3A-3D depict an AND logic gate—a genetic circuit that includes two constructs: one regulated by promoter P1, and one regulated by promoter P2. Transcription of the construct on the left, linked to P1, is activated in the presence of Input 1, while transcription of the construct on the right, linked to P2, is activated in the presence of Input 2. With this AND gate, the output molecule, STE protein, is only produced in the presence of Input 1 and Input 2 (FIG. 3A). In the presence of only Input 2 (FIG. 3B) or in the presence of only Input 1 (FIG. 3C), STE protein is not produced. Likewise, if neither Input 1 nor Input 2 is available, STE protein is not produced (FIG. 3D). In the presence of only Input 1, STE mRNA transcript is produced; however, the presence of the excised intronic miRNA prevents translation of STE mRNA and production of STE protein (FIG. 3C). In the presence of both Input 1 and Input 2, both the STE mRNA transcript and excised intronic miRNA are still produced; however, the excised intronic miRNA is “soaked up” by the decoy miRNA binding sites, the transcription of which is activated by Input 2. Thus, much of the STE mRNA is free from bound miRNA and can be translated to produce STE protein.

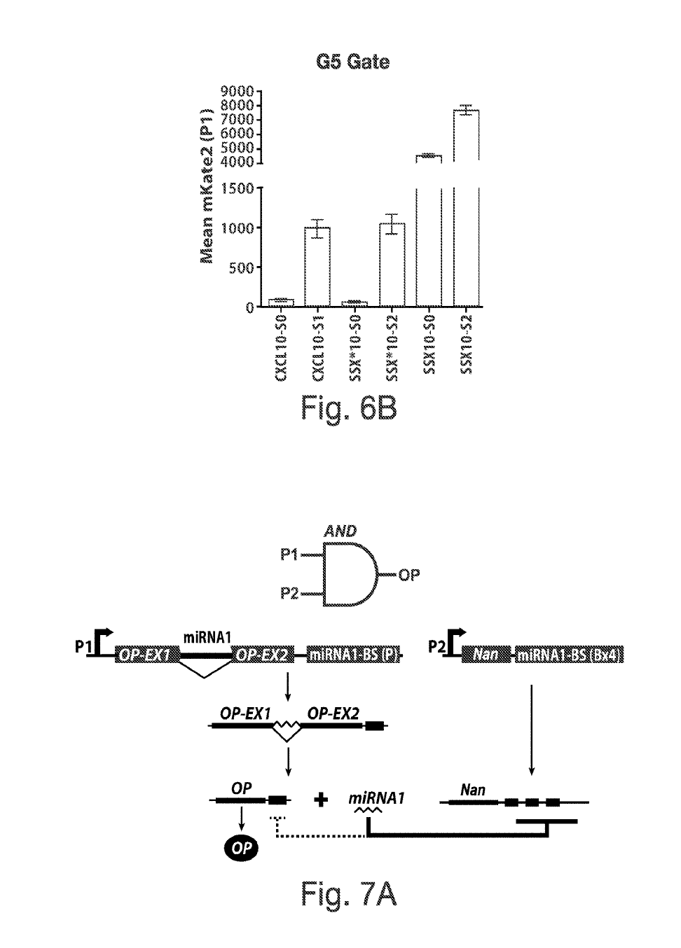

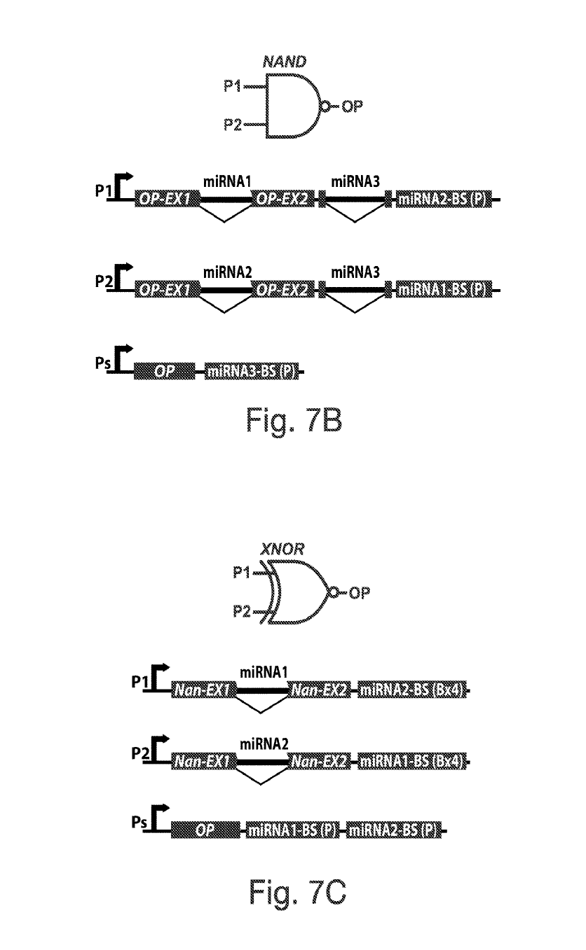

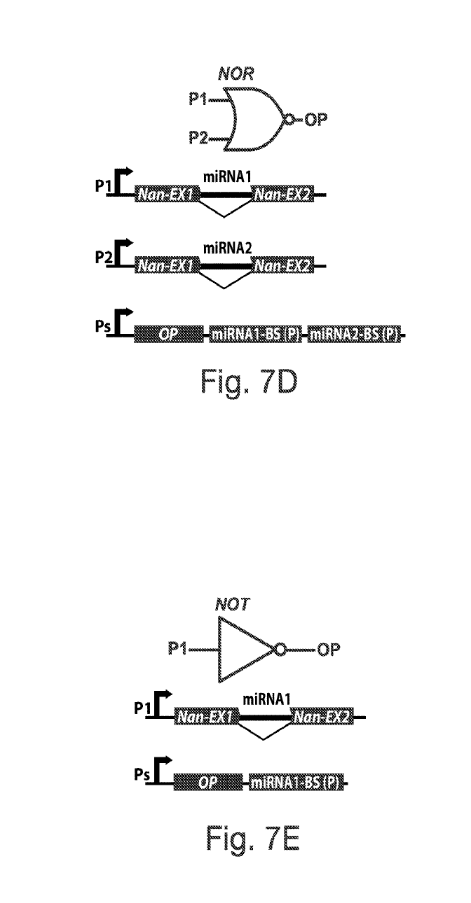

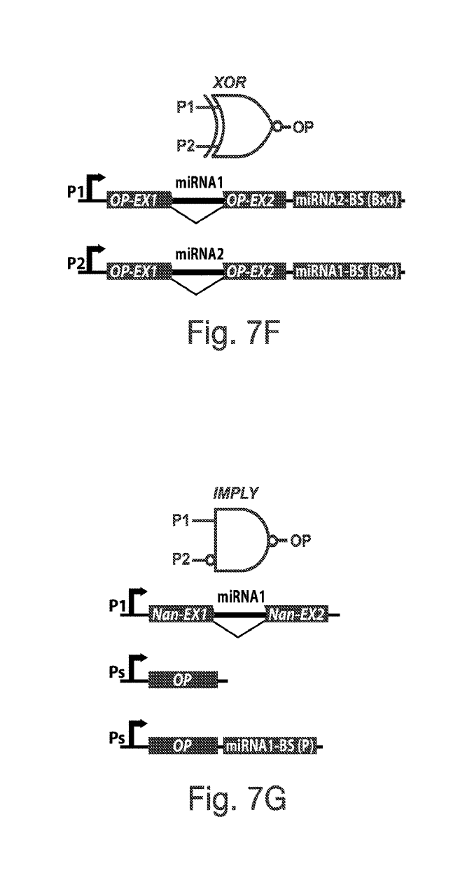

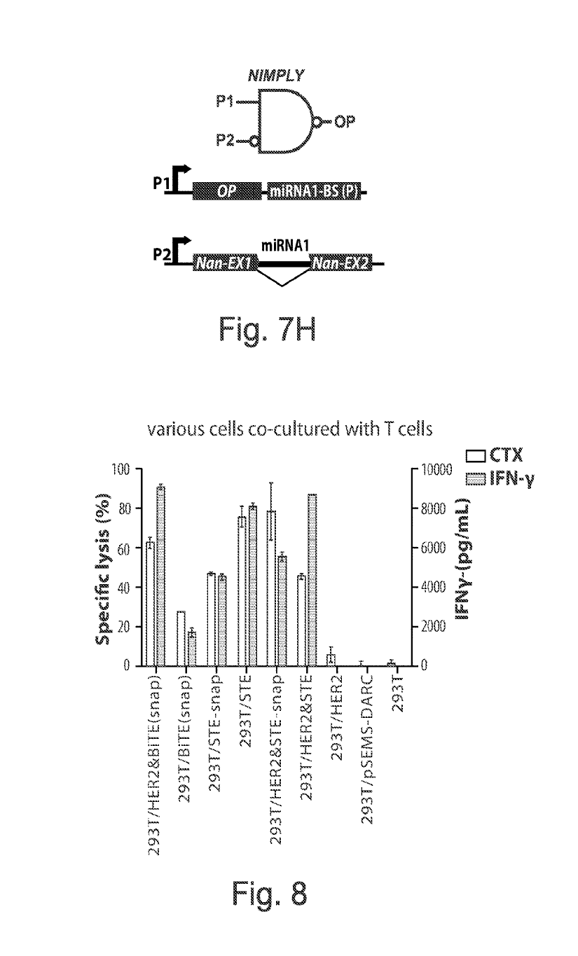

Other logic gates are depicted in FIGS. 7B-7H.

FIG. 7B depicts a logic gate comprising (a) a first nucleic acid comprising a promoter (P1) operably linked to (i) a nucleotide sequence encoding an output mRNA (OP-EX1-OB-EX2) containing an intronic miRNA (miRNA1), (ii) a nucleotide sequence encoding an intronic miRNA (miRNA3), and (iii) a nucleotide sequence encoding a miRNA binding site (miRNA2-BS (P)); (b) a second nucleic acid comprising a promoter (P2) operably linked to (i) a nucleotide sequence encoding an output mRNA (OP-EX1-OB-EX2) containing an intronic miRNA (miRNA2), (ii) a nucleotide sequence encoding an intronic miRNA (miRNA3) and (iii) a nucleotide sequence encoding a miRNA-BS (mirRNA1-BS (P)); and (c) a third nucleic acid comprising a promoter (Ps) operably linked to a nucleotide sequence encoding an output protein (OP) linked to a miRNA-BS (miRNA3-BS (P)), wherein miRNA1 is complementary to and binds to miRNA1-BS, miRNA3 is complementary to and binds to miRNA3-BS (P), and miRNA2 is complementary to and binds to miRNA2-BS (P).

FIG. 7C depicts a logic gate comprising (a) a first nucleic acid comprising a promoter (P1) operably linked to (i) a nucleotide sequence encoding a nascent RNA transcript (Nan-EX1-Nan-EX2) (e.g., a non-coding RNA transcript or and RNA transcript encoding a protein) containing an intronic miRNA (miRNA1) and (ii) a nucleotide sequence encoding four miRNA binding sites (miRNA2-BS (Bx4)); (b) a second nucleic acid comprising a promoter (P2) operably linked to (i) a nucleotide sequence encoding a nascent RNA transcript (Nan-EX1-Nan-EX2) containing an intronic miRNA (miRNA2), and (ii) a nucleotide sequence encoding four miRNA binding sites (miRNA1-BS (Bx4); and (c) a third nucleic acid comprising a promoter (Ps) operably linked to a nucleic acid encoding an output protein (OP) linked to (i) a first miRNA binding site (miRNA1-BS (P)) and (ii) a second miRNA binding site (miRNA2-BS (P)), wherein miRNA1 is complementary to and bind to miRNA1-BS (Bx4) and miRNA2 is complementary to and bind to miRNA2-BS (Bx4).

FIG. 7D depicts a logic gate comprising (a) a first nucleic acid comprising a promoter (P1) operably linked to a nucleotide sequence encoding a nascent RNA transcript (Nan-EX1-Nan-EX2) containing an intronic miRNA (miRNA1); (b) a second nucleic acid comprising a promoter (P2) operably linked to a nucleotide sequence encoding a nascent RNA transcript (Nan-EX1-Nan-EX2) containing an intronic miRNA (miRNA2); and (c) a third nucleic acid comprising a promoter (Ps) operably linked to a nucleic acid encoding an output protein (OP) linked to (i) a first miRNA binding site (miRNA1-BS (P)) and (ii) a second miRNA binding site (miRNA2-BS (P)), wherein miRNA1 is complementary to and binds to miRNA1-BS (P) and miRNA2 is complementary to and binds to miRNA2-BS.

FIG. 7E depicts a logic gate comprising (a) a first nucleic acid comprising a promoter (P1) operably linked to a nucleotide sequence encoding a nascent RNA transcript (Nan-EX1-Nan-EX2) containing an intronic microRNA (miRNA); and (b) a second nucleic acid comprising a promoter (Ps) operably linked to a nucleotide sequence encoding an output protein (OP) linked to a miRNA binding site (miRNA1-BS (P)), wherein miRNA1 is complementary to and binds to miRNA-BS (P).

FIG. 7F depicts a logic gate comprising (a) a first nucleic acid comprising a promoter (P1) operably linked to (i) a nucleotide sequence encoding an output mRNA (OP-EX1-OP-EX2) containing an intronic miRNA (miRNA1) and (ii) four miRNA binding sites (miRNA2-BS (Bx4)); and (b) a second nucleic acid comprising a promoter (P2) operably linked to (i) a nucleotide sequence encoding an output mRNA (OP-EX1-OP-EX2) containing an intronic miRNA (miRNA2) and (ii) four miRNA binding sites (miRNA1-BS (Bx4), wherein miRNA1 is complementary to and binds to miRNA1-BS (Bx4) and miRNA2 is complementary to and binds to miRNA2-BS (Bx4).

FIG. 7G depicts a logic gate comprising (a) a first nucleic acid comprising a promoter (P1) operably linked to a nucleotide sequence encoding a nascent RNA transcript (Nan-EX1-Nan-EX2) containing an intronic miRNA (miRNA1); (b) a second nucleic acid comprising a promoter (P2) operably linked to a nucleotide sequence encoding an output protein (OP); and (c) a third nucleic acid comprising a promoter (Ps) encoding an output protein (OP) linked to an miRNA binding site (miRNA1-BS (P), wherein miRNA1 is complementary to and binds to miRNA1-BS (P).

FIG. 7H depicts a logic gate comprising (a) a first nucleic acid comprising a promoter (P1) operably linked to a nucleotide sequence encoding an output protein (OP) linked to a miRNA binding site (miRNA1-BS); and (b) a second nucleic acid comprising a promoter (P2) operably linked to a nucleotide sequence encoding a nascent RNA transcript (Nan-EX1-Nan-EX2) containing an intronic miRNA (miRNA1), wherein miRNA1 is complementary to and binds to miRNA1-BS (P).

A “nucleic acid” is at least two nucleotides covalently linked together, and in some instances, may contain phosphodiester bonds (e.g., a phosphodiester “backbone”). An “engineered nucleic acid” (also referred to as a “construct”) is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing.

In some embodiments, a nucleic acid of the present disclosure is considered to be a nucleic acid analog, which may contain, at least in part, other backbones comprising, for example, phosphoramide, phosphorothioate, phosphorodithioate, O-methylphophoroamidite linkages and/or peptide nucleic acids. A nucleic acid may be single-stranded (ss) or double-stranded (ds), as specified, or may contain portions of both single-stranded and double-stranded sequence. In some embodiments, a nucleic acid may contain portions of triple-stranded sequence. A nucleic acid may be DNA, both genomic and/or cDNA, RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine.

Nucleic acids of the present disclosure may include one or more genetic elements. A “genetic element” refers to a particular nucleotide sequence that has a role in nucleic acid expression (e.g., promoter, enhancer, terminator) or encodes a discrete product of an engineered nucleic acid (e.g., a nucleotide sequence encoding a guide RNA, a protein and/or an RNA interference molecule, such as siRNA or miRNA).

Nucleic acids of the present disclosure may be produced using standard molecular biology methods (see, e.g., Green and Sambrook, Molecular Cloning, A Laboratory Manual, 2012, Cold Spring Harbor Press).

In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies. In some embodiments, an engineered nucleic acid is delivered to a cell on a vector. A “vector” refers to a nucleic acid (e.g., DNA) used as a vehicle to artificially carry genetic material (e.g., an engineered nucleic acid) into a cell where, for example, it can be replicated and/or expressed. In some embodiments, a vector is an episomal vector (see, e.g., Van Craenenbroeck K. et al. Eur. J. Biochem. 267, 5665, 2000, incorporated by reference herein). A non-limiting example of a vector is a plasmid (e.g., FIG. 3). Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. Another non-limiting example of a vector is a viral vector.

Thus, in some embodiments, engineered genetic circuits are delivered to cells (e.g., cancer cells) using a viral delivery system (e.g., retroviral, adenoviral, adeno-association, helper-dependent adenoviral systems, hybrid adenoviral systems, herpes simplex, pox virus, lentivirus, Epstein-Barr virus) or a non-viral delivery system (e.g., physical: naked DNA, DNA bombardment, electroporation, hydrodynamic, ultrasound or magnetofection; or chemical: cationic lipids, different cationic polymers or lipid polymer) (Nayerossadat N et al. Adv Biomed Res. 2012; 1: 27, incorporated herein by reference). In some embodiments, the non-viral based deliver system is a hydrogel-based delivery system (see, e.g., Brandl F, et al. Journal of Controlled Release, 2010, 142(2): 221-228, incorporated herein by reference).

A microRNA (“miRNA”) is a small non-coding RNA molecule (e.g., containing about 22 nucleotides) found in plants, animals, and some viruses, which typically functions under wild-type conditions in RNA silencing and post-transcriptional regulation of gene expression.

### Genetic Elements

Expression of engineered nucleic acids is driven by a promoter operably linked to a nucleic acid containing, for example, a nucleic acid encoding a molecule of interest. A “promoter” refers to a control region of a nucleic acid sequence at which initiation and rate of transcription of the remainder of a nucleic acid sequence are controlled. A promoter drives expression or drives transcription of the nucleic acid sequence that it regulates. A promoter may also contain sub-regions at which regulatory proteins and molecules may bind, such as RNA polymerase and other transcription factors. Promoters may be constitutive, inducible, activatable, repressible, tissue-specific or any combination thereof.

Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.”

In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see U.S. Pat. No. 4,683,202 and U.S. Pat. No. 5,928,906).

In some embodiments, a promoter is an “inducible promoter,” which refer to a promoter that is characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

The administration or removal of an inducer signal results in a switch between activation and inactivation of the transcription of the operably linked nucleic acid sequence. Thus, the active state of a promoter operably linked to a nucleic acid sequence refers to the state when the promoter is actively regulating transcription of the nucleic acid sequence (i.e., the linked nucleic acid sequence is expressed). Conversely, the inactive state of a promoter operably linked to a nucleic acid sequence refers to the state when the promoter is not actively regulating transcription of the nucleic acid sequence (i.e., the linked nucleic acid sequence is not expressed).

An inducible promoter of the present disclosure may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). An extrinsic inducer signal or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Inducible promoters of the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some embodiments, an inducer signal of the present disclosure is isopropyl β-D-1-thiogalactopyranoside (IPTG), which is a molecular mimic of allolactose, a lactose metabolite that triggers transcription of the lac operon, and it is therefore used to induce protein expression where the gene is under the control of the lac operator. IPTG binds to the lac repressor and releases the tetrameric repressor from the lac operator in an allosteric manner, thereby allowing the transcription of genes in the lac operon, such as the gene coding for beta-galactosidase, a hydrolase enzyme that catalyzes the hydrolysis of β-galactosides into monosaccharides. The sulfur (S) atom creates a chemical bond which is non-hydrolyzable by the cell, preventing the cell from metabolizing or degrading the inducer. IPTG is an effective inducer of protein expression, for example, in the concentration range of 100 μM to 1.0 mM. Concentration used depends on the strength of induction required, as well as the genotype of cells or plasmid used. If lacIq, a mutant that over-produces the lac repressor, is present, then a higher concentration of IPTG may be necessary. In blue-white screen, IPTG is used together with X-gal. Blue-white screen allows colonies that have been transformed with the recombinant plasmid rather than a non-recombinant one to be identified in cloning experiments.

Other inducible promoter systems are known in the art and may be used in accordance with the present disclosure.

### Immunomodulatory Agents

An immunomodulatory agent is an agent (e.g., protein) that regulates an immune response. The present disclosure provides, in some embodiments, engineered genetic circuits that include nucleic acids encoding immunomodulatory agents that are expressed at the surface of, or secreted from, a cancerous cell or secreted from a cancerous cell.

In some embodiments, the immunomodulatory agent is a synthetic T cell engager (STE). A “synthetic T cell engager” is a molecule (e.g., protein) that binds to (e.g., through a ligand-receptor binding interaction) a molecule on the surface of a T cell (e.g., a cytotoxic T cell), or otherwise elicits a cytotoxic T cell response. In some embodiments, an STE is a receptor that binds to a ligand on the surface of a T cell. In some embodiments, an STE is an anti-CD3 antibody or antibody fragment. A STE of the present disclosure is typically expressed at the surface of, or secreted from, a cancer cell or other disease cell to which a nucleic acid encoding the STEs is delivered.

Examples of STEs of the present disclosure include antibodies, antibody fragments and receptors that binds to T cell surface antigens. T cell surface antigens include, for example, CD3, CD4, CD 8 and CD45. STEs expressed by the genetic circuits of the present disclosure may also be selected from any of the immunomodulatory agents described below.

In some embodiments, a genetic circuit of the present disclosure modulates expression of a chemokine, a cytokine or a checkpoint inhibitor.

Immunomodulatory agents include immunostimulatory agents and immunoinhibitory agents. As used herein, an immunostimulatory agent is an agent that stimulates an immune response (including enhancing a pre-existing immune response) in a subject to whom it is administered, whether alone or in combination with another agent. Examples include antigens, adjuvants (e.g., TLR ligands such as imiquimod, imidazoquinoline, nucleic acids comprising an unmethylated CpG dinucleotide, monophosphoryl lipid A or other lipopolysaccharide derivatives, single-stranded or double-stranded RNA, flagellin, muramyl dipeptide), cytokines including interleukins (e.g., IL-2, IL-7, IL-15 (or superagonist/mutant forms of these cytokines), IL-12, IFN-gamma, IFN-alpha, GM-CSF, FLT3-ligand, etc.), immunostimulatory antibodies (e.g., anti-CTLA-4, anti-CD28, anti-CD3, or single chain/antibody fragments of these molecules), and the like.

As used herein, an immunoinhibitory agent is an agent that inhibits an immune response in a subject to whom it is administered, whether alone or in combination with another agent. Examples include steroids, retinoic acid, dexamethasone, cyclophosphamide, anti-CD3 antibody or antibody fragment, and other immunosuppressants.

Antigens may be, without limitation, a cancer antigen, a self-antigen, a microbial antigen, an allergen, or an environmental antigen. An antigen may be peptide, lipid, or carbohydrate in nature, but it is not so limited.

A cancer antigen is an antigen that is expressed preferentially by cancer cells (e.g., it is expressed at higher levels in cancer cells than on non-cancer cells) and in some instances it is expressed solely by cancer cells. The cancer antigen may be expressed within a cancer cell or on the surface of the cancer cell. The cancer antigen may be MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5). The cancer antigen may be selected from the group consisting of GAGE-1, GAGE-2, GAGE-3, GAGE-4, GAGE-5, GAGE-6, GAGE-7, GAGE-8, GAGE-9. The cancer antigen may be selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pmel117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, and c-erbB-2.

### Cells and Cell Expression

Engineered genetic circuits of the present disclosure are typically delivered systemically and activated (transcription of the circuits are activated) conditionally (based on the presence or absence of input signals) in a particular cell type, such as a cancerous cell, a benign tumor cell or other disease cell. Thus, in some embodiments, genetic circuits (logic gates) are delivered to a subject having tumor cells or cancer cells, and the genetic circuits (logic gates) are expressed in the tumor cells or cancer cells.

A cancerous cell may be any type of cancerous cell, including, but not limited to, premalignant neoplasms, malignant tumors, metastases, or any disease or disorder characterized by uncontrolled cell growth such that it would be considered cancerous or precancerous. The cancer may be a primary or metastatic cancer. Cancers include, but are not limited to, ocular cancer, biliary tract cancer, bladder cancer, pleura cancer, stomach cancer, ovary cancer, meninges cancer, kidney cancer, brain cancer including glioblastomas and medulloblastomas, breast cancer, cervical cancer, choriocarcinoma, colon cancer, endometrial cancer, esophageal cancer, gastric cancer, hematological neoplasms including acute lymphocytic and myelogenous leukemia, multiple myeloma, AIDS-associated leukemias and adult T-cell leukemia lymphoma, intraepithelial neoplasms including Bowen's disease and Paget's disease, liver cancer, lung cancer, lymphomas including Hodgkin's disease and lymphocytic lymphomas, neuroblastomas, oral cancer including squamous cell carcinoma, ovarian cancer including those arising from epithelial cells, stromal cells, germ cells and mesenchymal cells, pancreatic cancer, prostate cancer, rectal cancer, sarcomas including leiomyosarcoma, rhabdomyosarcoma, liposarcoma, fibrosarcoma, and osteosarcoma, skin cancer including melanoma, Kaposi's sarcoma, basocellular cancer, and squamous cell cancer, testicular cancer including germinal tumors such as seminoma, non-seminoma, teratomas, choriocarcinomas, stromal tumors and germ cell tumors, thyroid cancer including thyroid adenocarcinoma and medullar carcinoma, and renal cancer including adenocarcinoma and Wilms' tumor. Commonly encountered cancers include breast, prostate, lung, ovarian, colorectal, and brain cancer. In some embodiments, the tumor is a melanoma, carcinoma, sarcoma, or lymphoma.

Engineered nucleic acids of the present disclosure may be expressed in a broad range of host cell types. In some embodiments, engineered nucleic acids are expressed in mammalian cells (e.g., human cells), bacterial cells (Escherichia coli cells), yeast cells, insect cells, or other types of cells. Engineered nucleic acids of the present disclosure may be expressed in vivo, e.g., in a subject such as a human subject.

In some embodiments, engineered nucleic acids are expressed in mammalian cells. For example, in some embodiments, engineered nucleic acids are expressed in human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, engineered nucleic acids are expressed in human embryonic kidney (HEK) cells (e.g., HEK 293 or HEK 293T cells). In some embodiments, engineered nucleic acids are expressed in stem cells (e.g., human stem cells) such as, for example, pluripotent stem cells (e.g., human pluripotent stem cells including human induced pluripotent stem cells (hiPSCs)). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Cells of the present disclosure, in some embodiments, are modified. A modified cell is a cell that contains an exogenous nucleic acid or a nucleic acid that does not occur in nature. In some embodiments, a modified cell contains a mutation in a genomic nucleic acid. In some embodiments, a modified cell contains an exogenous independently replicating nucleic acid (e.g., an engineered nucleic acid present on an episomal vector). In some embodiments, a modified cell is produced by introducing a foreign or exogenous nucleic acid into a cell. A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), transduction, conjugation, or microinjection of purified DNA directly into the nucleus of the cell (see, e.g., Capecchi M. R. Cell. 1980 November; 22(2 Pt 2): 479-88).

In some embodiments, a cell is modified to express a reporter molecule. In some embodiments, a cell is modified to express an inducible promoter operably linked to a reporter molecule (e.g., a fluorescent protein such as green fluorescent protein (GFP) or other reporter molecule).

In some embodiments, a cell is modified to overexpress an endogenous protein of interest (e.g., via introducing or modifying a promoter or other regulatory element near the endogenous gene that encodes the protein of interest to increase its expression level). In some embodiments, a cell is modified by mutagenesis. In some embodiments, a cell is modified by introducing an engineered nucleic acid into the cell in order to produce a genetic change of interest (e.g., via insertion or homologous recombination).

In some embodiments, an engineered nucleic acid may be codon-optimized, for example, for expression in mammalian cells (e.g., human cells) or other types of cells. Codon optimization is a technique to maximize the protein expression in living organism by increasing the translational efficiency of gene of interest by transforming a DNA sequence of nucleotides of one species into a DNA sequence of nucleotides of another species. Methods of codon optimization are well-known.

Engineered nucleic acids of the present disclosure may be transiently expressed or stably expressed. “Transient cell expression” refers to expression by a cell of a nucleic acid that is not integrated into the nuclear genome of the cell. By comparison, “stable cell expression” refers to expression by a cell of a nucleic acid that remains in the nuclear genome of the cell and its daughter cells. Typically, to achieve stable cell expression, a cell is co-transfected with a marker gene and an exogenous nucleic acid (e.g., engineered nucleic acid) that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. Examples of marker genes and selection agents for use in accordance with the present disclosure include, without limitation, dihydrofolate reductase with methotrexate, glutamine synthetase with methionine sulphoximine, hygromycin phosphotransferase with hygromycin, puromycin N-acetyltransferase with puromycin, and neomycin phosphotransferase with Geneticin, also known as G418. Other marker genes/selection agents are contemplated herein.

Expression of nucleic acids in transiently-transfected and/or stably-transfected cells may be constitutive or inducible. Inducible promoters for use as provided herein are described above.

Some aspects of the present disclosure provide cells that comprises 1 to 10 engineered nucleic acids. In some embodiments, a cell comprises 1, 2, 3, 4, 5, 6, 7, 8, 9, 10 or more engineered nucleic acids. It should be understood that a cell that “comprises an engineered nucleic acid” is a cell that comprises copies (more than one) of an engineered nucleic acid. Thus, a cell that “comprises at least two engineered nucleic acids” is a cell that comprises copies of a first engineered nucleic acid and copies of an engineered second nucleic acid, wherein the first engineered nucleic acid is different from the second engineered nucleic acid. Two engineered nucleic acids may differ from each other with respect to, for example, sequence composition (e.g., type, number and arrangement of nucleotides), length, or a combination of sequence composition and length. For example, the SDS sequences of two engineered nucleic acids in the same cells may differ from each other.

Some aspects of the present disclosure provide cells that comprises 1 to 10 episomal vectors, or more, each vector comprising, for example, an engineered nucleic acids. In some embodiments, a cell comprises 1, 2, 3, 4, 5, 6, 7, 8, 9, 10 or more vectors.

Also provided herein, in some aspects, are methods that comprise introducing into a cell an (e.g., at least one, at least two, at least three, or more) engineered nucleic acid or an episomal vector (e.g., comprising an engineered nucleic acid). As discussed elsewhere herein, an engineered nucleic acid may be introduced into a cell by conventional methods, such as, for example, electroporation, chemical (e.g., calcium phosphate or lipid) transfection, fusion with bacterial protoplasts containing recombinant plasmids, transduction, conjugation, or microinjection of purified DNA directly into the nucleus of the cell.

### In Vivo Delivery

Engineered nucleic acids of the present disclosure may be delivered to a subject (e.g., a mammalian subject, such as a human subject) by any in vivo delivery method known in the art. For example, engineered nucleic acids may be delivered intravenously. In some embodiments, engineered nucleic acids are delivered in a delivery vehicle (e.g., non-liposomal nanoparticle or liposome). In some embodiments, engineered genetic circuits are delivered systemically to a subject having a cancer or other disease and activated (transcription is activated) specifically in cancer cells or diseased cells of the subject.

Engineered genetic circuits, as discussed above, may be delivered to cells (e.g., cancer cells) of a subject using a viral delivery system (e.g., retroviral, adenoviral, adeno-association, helper-dependent adenoviral systems, hybrid adenoviral systems, herpes simplex, pox virus, lentivirus, Epstein-Barr virus) or a non-viral delivery system (e.g., physical: naked DNA, DNA bombardment, electroporation, hydrodynamic, ultrasound or magnetofection; or chemical: cationic lipids, different cationic polymers or lipid polymer) (Nayerossadat N et al. Adv Biomed Res. 2012; 1: 27, incorporated herein by reference). In some embodiments, the non-viral based deliver system is a hydrogel-based delivery system (see, e.g., Brandl F, et al. Journal of Controlled Release, 2010, 142(2): 221-228, incorporated herein by reference).

### Synthetic Promoter Libraries

Synthetic promoter libraries are provided that include a plurality of nucleic acids, wherein each nucleic acid in the library comprises a synthetic promoter sequence. Three designs for synthetic promoter libraries are provided. In two of the designs (“Design 1” and “Design 2”), the promoter sequences of the library comprise 8 mer nucleotide sequences that are joined in tandem (head-to-tail). In one of these designs (“Design 2”), 3 mer nucleotide spacers are placed in between each pair of 8 mer nucleotide sequences. In the third design (“Design 3”), the nucleic acid sequences of the library comprise 11 mer nucleotide sequences that are joined in tandem (head-to-tail), with 3 mer nucleotide spacers placed in between each pair of 11 mer nucleotide sequences.

The number of 8 mer or 11 mer nucleotide sequences in tandem can be at least: 2, 3, 4, 5, 6, 7, 8, 9, 10, 11, 12, 13, 14, 15, 16, 17, 18, 19, or 20 8 mer or 11 mer nucleotide sequences. The sequence of each 8 mer or 11 mer nucleotide sequence in a nucleic acid can be random (i.e., the sequence, wherein each N represents any nucleotide) and the 8 mer or 11 mer nucleotide sequences in any nucleic acid can be randomly selected so that the plurality of nucleic acids in the promoter library represents substantially all possible sequences or all possible sequences of the length of the nucleic acid that is selected for the library. Alternatively, if a particular nucleotide sequences or compositions (e.g., pyrimidine content) are to be favored or required, or disfavored or avoided, then the 8 mer or 11 mer nucleotide sequences can be designed to have certain nucleotides in certain positions, or certain nucleotide content, as desired. In such cases, the plurality of nucleic acids in the promoter library represents a selected subset of all possible sequences.

In some embodiments, a nucleotide spacer of defined sequence is placed between each 8 mer or 11 mer nucleotide sequence. The nucleotide spacer preferably is a 3 mer nucleotide, but other length spacers can be used, such as 1, 2, 4, or 5 nucleotides. The 3 mer nucleotide spacers in some embodiments are selected from AGC, ATC, GAC, ACT, AGT, GTC, GAT, and GCT. In some embodiments, each nucleotide spacer used in a nucleic acid in the library is different than other nucleotide spacers in the same nucleic acid.

In some embodiments, the nucleic acids in the synthetic promoter library further includes restriction endonuclease sites at the 5′ and 3′ ends. In some embodiments, the restriction endonuclease site at the 5′ end is a SbfI site and the restriction endonuclease site at the 3′ end is an AscI site. Other restriction endonuclease sites may be used.

In some embodiments, each of the nucleic acids in the synthetic promoter library further includes a nucleotide sequence encoding an output molecule operably linked to the promoter sequence. The output molecule in some embodiments is a detectable molecule, such as a fluorescent or colored protein (e.g., mKate2), an enzyme, or any other type of detectable nucleic acid or polypeptide known in the art.

The synthetic promoter libraries can be used in method of selecting synthetic promoters. The method includes obtaining a library comprising nucleic acid molecules comprising synthetic promoter sequences operably linked to an output molecule, expressing the library in one or more types of cells, detecting the expression of the output molecule, and isolating the cells in which the output molecule is expressed. Optionally the method also includes determining the sequence of the synthetic promoter sequences in the isolated cells.

In some embodiments, the one or more types of cells are at least two different types of cells, such as cancer cells and matched non-cancer cells, such as ovarian cancer cells and ovarian cells, or breast cancer cells and breast cells, etc.

By comparing the synthetic promoter sequences that drive the expression of the output molecule in each of the at least two different types of cells, synthetic promoter sequences that are more active in one of the at least two different types of cells than in another of the at least two different types of cells can be identified. Thus if the at least two different types of cells are cancer cells and non-cancer cells, then promoters can be identified that are active in cancer cells but not in non-cancer cells, or vice versa.

By “more active in one of the at least two different types of cells than in another of the at least two different types of cells” is meant that the promoter has at least 10%, 50%, 100%, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 500-fold, or 1000-fold (or even more) greater activity in one of the two types of cells. For example, a synthetic promoter isolated from a library by these methods can be essentially inactive in one type of cell and active in another type of cell, which provides cell type-specific synthetic promoters.

### EXAMPLES

### Example 1

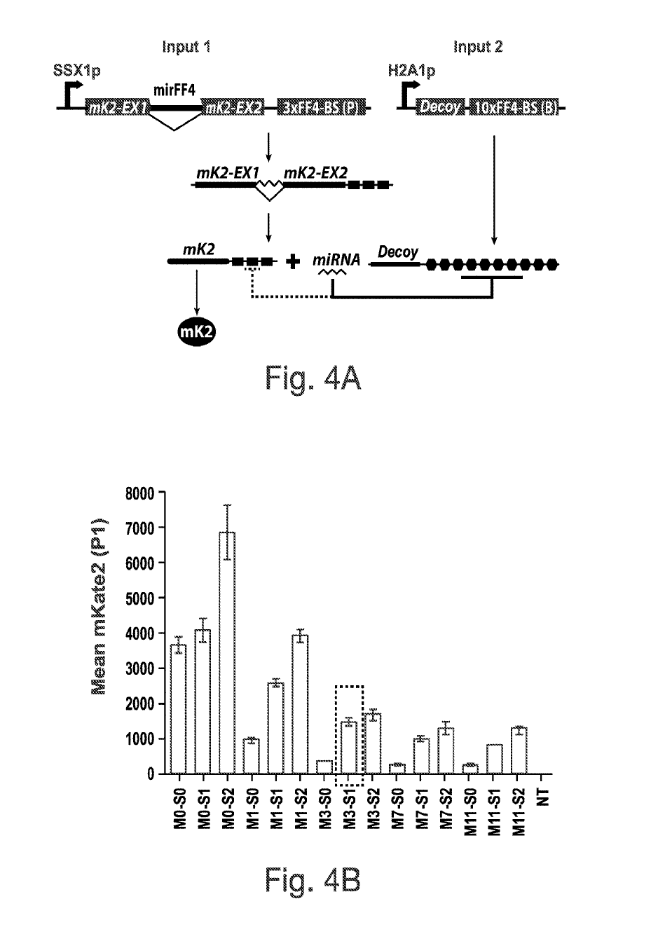

Two human promoters were used as promoter inputs for the engineered genetic circuit used in this Example. These human promoters, SSX1 (Input 1) and H2A1 (Input 2), are over-expressed in many human cancers (Input 1 encodes the mKate2 output containing an intervening mirFF4) (FIG. 4A). The mKate2 output levels were measured for different circuit configurations, with respect to (a) the number of perfect-match mirFF4 binding sites (FF4-BS) encoded in Input 1 (downstream of mK2) and (b) two different configurations for the “sponge” construct in Input 2. FIG. 4B, x-axis annotations: M# represents Input 1 with the number of mirFF4 binding sites (FF4-BS) encoded downstream from mKate2/mirFF4. For example, M3 represents Input 1 with 3 perfect-match mirFF4 binding sites (FF4-BS) (FIG. 4A). S0, S1 and S2 represent three different sponge/Input 2 configurations. S0 is a negative control transcript with no mirFF4 binding sites. S1 is a Decoy transcript with 10 bulged mirFF4 binding sites encoded at the 3′ end of the construct (FIG. 4A). S2 is similar to S1, but with an additional circular intron with 10 bulged FF4-BS located upstream from the 10 bulged mirFF4-BS encoded at the 3′ end of the construct. The engineered genetic circuit (logic gate) depicted in FIG. 4A corresponds to M3-S1 in FIG. 4B (highlighted by a dashed box). The results are represented in mean mKate2 expression (P1), which is the average mKate2 for cells gated for SSC/FSC in FACS to remove cell clumps and debris. Error bars represent SEM. NT represents non-transfected cells.

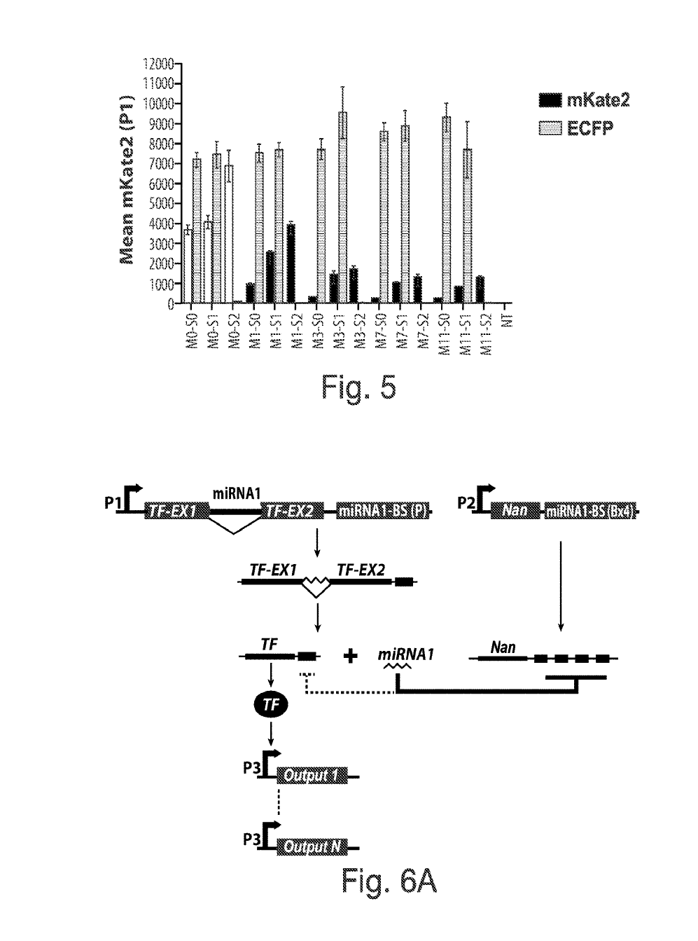

The experiment was repeated with ECFP labeling (FIG. 5).

### Example 2

The engineered genetic circuit (G5) described in this Example is based on the circuit (AND gate) encoding mKate2, described in Example 1, with the exception that the AND gate product is not mKate2, but rather a synthetic transcription factor (annotated “TF” in FIG. 6A). In this example, the TF is the fusion protein GAL4BD-VP16 AD (the yeast GALA DNA binding domain fused to the viral VP16 transcription activation domain), although it can be any transcription activator such as rtTA3, TALE-TFs and ZF-TFs. Alternatively, this can also be a transcriptional repressor such as GAL4BD-KRAB. Because the output is a transcription factor rather than a reporter/effector protein, it can regulate the expression of multiple outputs encoded downstream from the TF target promoter. In this example, the target promoter (annotated P3) is the synthetic G5 promoter that consists of a minimal viral or human promoter with 5 upstream GAL4 DNA binding sites. The I/O curve of this synthetic promoter can be tuned with the number of the GALA binding sites. Therefore, the ratio between any multiple outputs, together with the activation threshold for each output can be determined by the number of GALA binding sites in the synthetic P3 promoter.

FIG. 6B shows experimental results. CXCL10 is CXCL1p regulating a GAL4BD-VP16AD harboring a mirFF4v2B intron and 10 downstream mirFF4-Bs. SSX10 is SSX1p regulating a GAL4BD-VP16AD harboring a mirFF4v2B intron and 10 downstream mirFF4-Bs. SSX*10 is truncated SSX1p in which part of the 5′ UTR was removed together with the KOZAK sequence, regulating a GAL4BD-VP16AD harboring a mirFF4v2B intron and 10 downstream mirFF4-Bs. Sponge S0 is a negative control transcript mirFF4-BS. Sponge S2 is Decoy transcript with 10 bulged FF4-BS encoded on the 3′ end, with an additional circular intron with 10 bulged mirFF4-BS located upstream to the 10 bulged mirFF4-BS which are encoded in the transcript3′. In all samples, the mKate2 output is encoded under a Gyp.

### Example 3

### BiTE and STE Trigger Robust Tumor Killing HEK-293T Cells

Anti-HER2 bispecific T cell engager (BiTE) and surface T cell engager (STE) trigger T cells to mediate robust tumor killing and IFN-γ secretion (FIG. 8). HEK-293T (minimally expressing HER2) cells were transfected with various DNA constructs as indicated. 48 hours post transfection, various HEK-293T cells were harvested and co-cultured with human T cells for 5 hrs or 24 hrs. 5 hr cytotoxicity by T cells was measured by lactate dehydrogenase (LDH) release assay (Korzeniewski C and Callewaert D M, Journal of Immunological Methods, 1983, 64(3):313-320, incorporated herein by reference) and 24 hr IFN-γ secretion by T cells was measured by IFN-γ ELISA. Data show that T cells mediate robust tumor killing and IFN-γ secretion on BiTE secreting tumor cells (group 1-2). The tumor killing and IFN-γ secretion correlate with HER2 expression level on tumor cells (group 1-2). T cells also mediate robust tumor killing and IFN-γ secretion on STE expressing tumor cells (group 3-6), and the cytotoxicity and IFN-γ secretion are independent of tumor antigen (HER2) expression (group 3-6). Furthermore, T cells mediate minimal tumor killing and IFN-γ secretion when co-cultured with HEK-293T cells expressing non-BiTE and non-STE control proteins (group 7-9).

### Stable 4T1 Cells

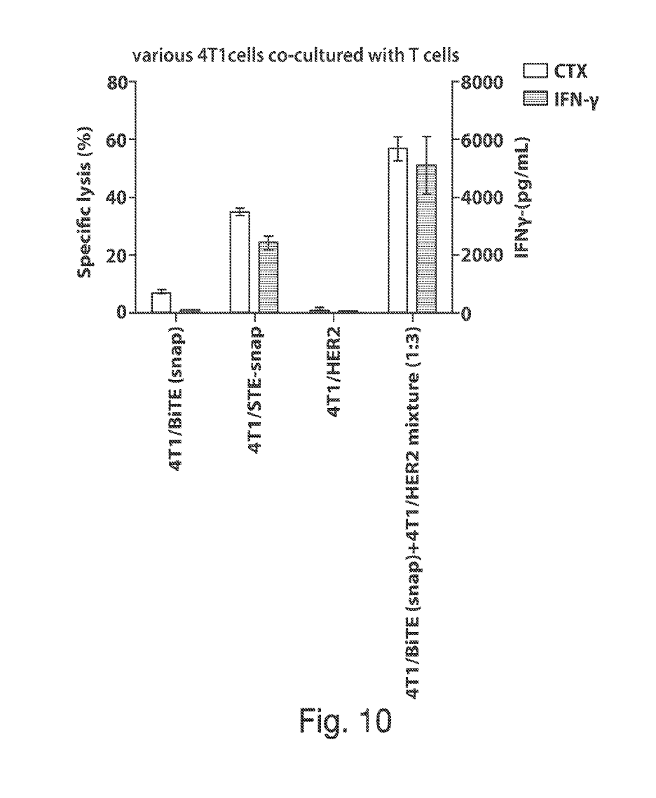

Stable 4T1 cells (HER2-) expressing indicated DNA constructs (STRICT017 +018) were co-cultured with human T cells for 5 hrs or 24 hrs (FIG. 10). 5 hr cytotoxicity by T cells was measured by LDH release assay and 24 hr IFN-γ secretion by T cells was measured by IFN-γ ELISA (FIG. 10A). Data show that T cells mediate minimal killing and IFN-γ secretion on HER2- or STE-tumor cells (groups 1 and 3). T cells mediate robust tumor killing and IFN-γ secretion on STE-expressing tumor cells (group 2). T cells also mediate robust tumor killing and IFN-γ secretion when co-cultured with cell mixtures consisting of low numbers of BiTE secreting cells with non-BiTE secreting tumor. This indicates minimal numbers of BiTE secreting cells in the tumor mass can elicit robust tumor mass killing and IFN-γ release (group 4).

### Stable HEK-293T Cells

Stable HEK-293T cells (minimally expressing HER2) expressing indicated DNA constructs were co-cultured with human T cells for 5 hrs or 24 hrs. 5 hr cytotoxicity by T cells was measured by LDH release assay and 24 hr IFN-γ secretion by T cells was measured by IFN-γ ELISA (FIG. 10B). Data show that T cells mediate minimal killing and IFN-γ secretion on BiTE- or STE-tumor cells (group 4). T cells mediate robust cytotoxicity and IFN-γ secretion on BiTE secreting tumor cells (group 1). T cells also mediate robust cytotoxicity and IFN-γ secretion on STE-expressing tumor cells (groups 2 and 3). Furthermore, T cells also mediate robust tumor killing and IFN-γ secretion when co-cultured with cell mixtures consisting of low numbers of BiTE secreting cells with non-BiTE secreting tumor cells. This indicates minimal numbers of BiTE secreting cells in the tumor mass can elicit robust tumor mass killing and IFN-γ release (group 5 & 6).

### Stable MDA-MB452 (HER2+) Cells (Human Breast Cancer Cell Line)

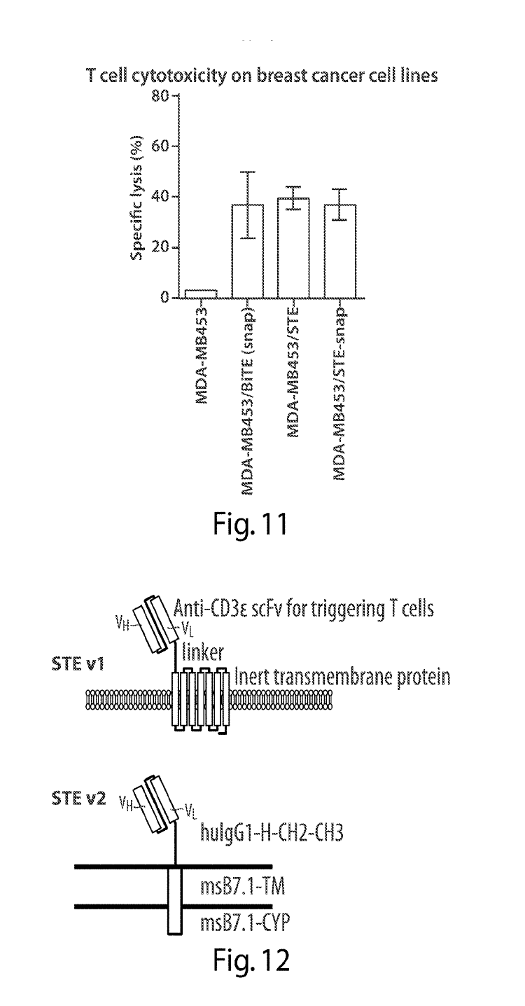

Anti-HER2 bispecific T cell engager (BiTE) and surface T cell engager (STE) trigger T cells to mediate robust tumor killing on human breast cancer cell line (FIG. 11). Stable MDA-MB453 (HER2+) cell lines were created by lentiviral transduction with various DNA constructs (STRICT034, 035) as indicated. Donor #2 T cells were used. The E:T ratio was 10:1; 6×10⁵:6×10⁴. Various MDA-MB453 cells were harvested and co-cultured with human T cells for 5 hrs. 5 hr cytotoxicity by T cells was measured by LDH release assay. Data show that T cells mediate robust tumor killing on BiTE secreting tumor cells (group 2). T cells also mediate robust tumor killing on STE expressing tumor cells (group 3-4). Furthermore, T cells mediate minimal tumor killing when co-cultured with parental MDA-MB453 tumor cell line (group 1).

### Example 4

### T Cells Kill Doxycycline-Induced STE-Expressing Cells Efficiently

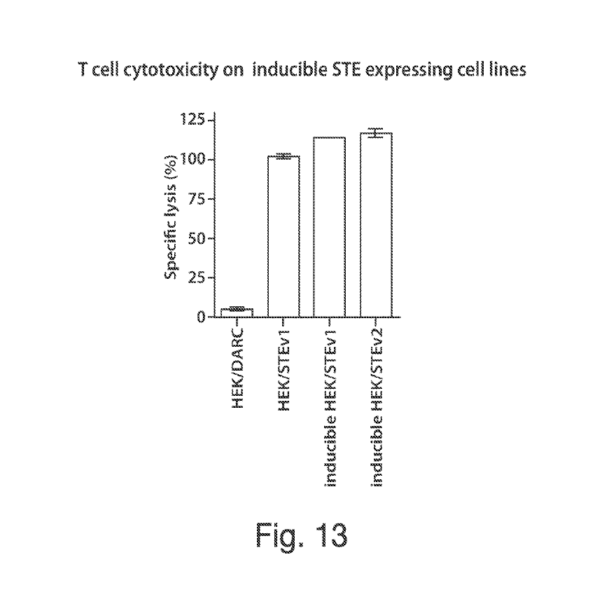

Surface T cell engager (STE) version 1 (v1) and version 2 (v2) both trigger T cells to mediate robust tumor killing on HEK-293T cells (FIG. 13). Various inducible STE expressing HEK-293T cell lines were created by lentiviral transduction. Various HEK-293T cells were harvested and co-cultured with human T cells for 5 hrs. 5 hr cytotoxicity by T cells was measured by LDH release assay. Data show that T cells mediate robust tumor killing on transfected STEv1 expressing tumor cells (column 2). T cells also mediate robust tumor killing on inducible STEv1 and STEv2 expressing tumor cells (columns 3 and 4). Furthermore, T cells mediate minimal tumor killing when co-cultured with non-STE expressing HEK-293T cell line (column 1).

### Example 5

### Increase in T Cell Killing Efficiency of Tumor Cells HEK-293T Cells

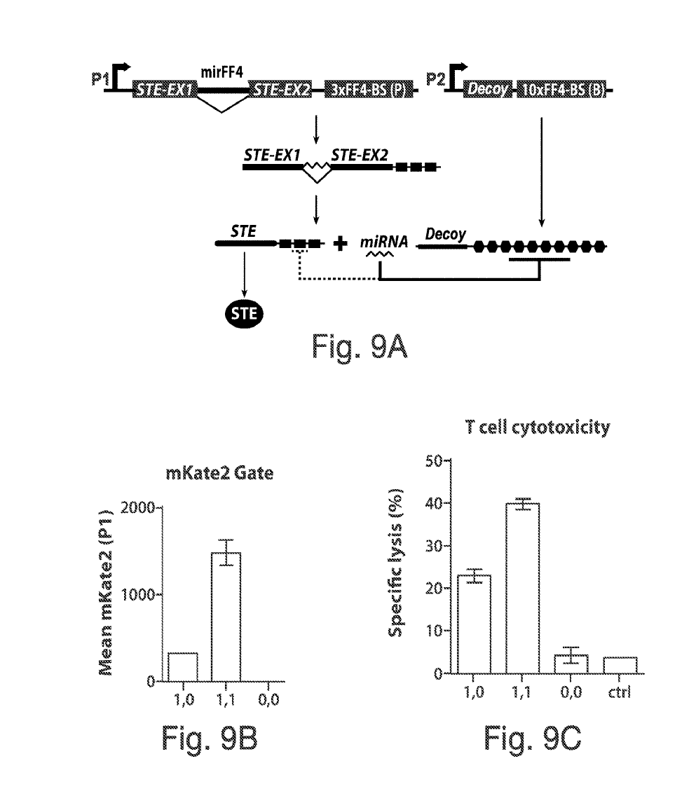

AND gate architecture was harnessed increase the T cell killing efficiency of tumor cells (FIG. 9). HEK-293T cells were transfected with various DNA constructs (STRICT014) as indicated (FIG. 9A) and Donor #S T cells were used. The E:T ratio was 10:1; 6×10⁵:6×10⁴. For the right panel (FIG. 9B), (1,0) indicated cells transfected with STE only). (1,1) indicated cells transfected with STE and sponge. (0,0) indicated cells transfected with a non-STE protein. Ctrl indicated non-transfected cells. 48 hrs post transfection, various HEK-293T cells were harvested and co-cultured with human T cells for 5 hrs. Cytotoxicity by T cells was measured by LDH release assay. Data show that T cells kill 293T/STE expressing cells (column 1) and the killing can be greatly enhanced by the AND gate architecture (column 2). T cells exhibit minimal killing on not STE expressing cells (column 3 & 4). For the left panel (FIG. 9C), the Input 2 condition was not tested since it does not encode the output protein. (0,0) represents non-transfected cells. An additional experiment is conducted to further decrease the output of the AND gate at state (1,0) by removing the Kozak sequence and the 5′ UTR of SSX1 promoter.

### HEK-293T Cells (GAL4 Gate v1 for STE)

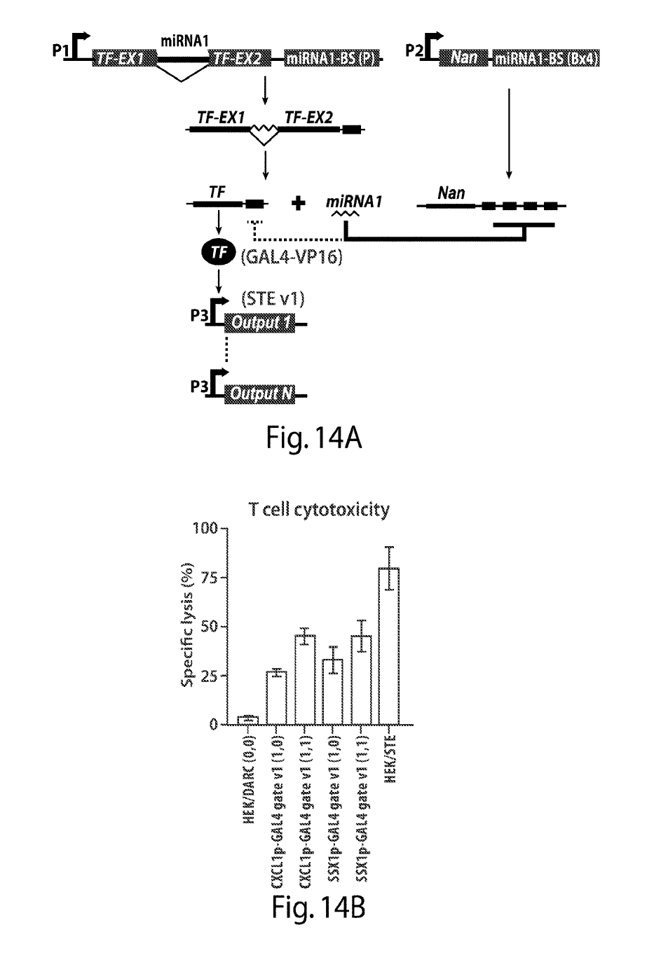

HEK-293T cells were transfected with various DNA constructs (STRICT037, 039, 040) as indicated and Donor #2's T cells were used (FIG. 14). The E:T ratio was 10:1; 6×10⁵:6×10⁴. The left panel showed the circuit used for this T cell cytotoxicity experiment (FIG. 14A). In the right panel, (1,0) indicated cells transfected with STE only. (1,1) indicated cells transfected with STE and sponge. (0,0) indicated cells transfected with a non-STE protein. 48 hrs post transfection, various HEK-293T cells were harvested and co-cultured with human T cells for 5 hrs. Cytotoxicity by T cells was measured by LDH release assay (FIG. 14B). Data show that T cells kill STE expressing (1,0) cells (column 2 and 4) and the killing can be greatly enhanced by the AND gate (1,1) architecture (column 3 and 5). T cells exhibit minimal killing on not STE expressing cells (column 1).

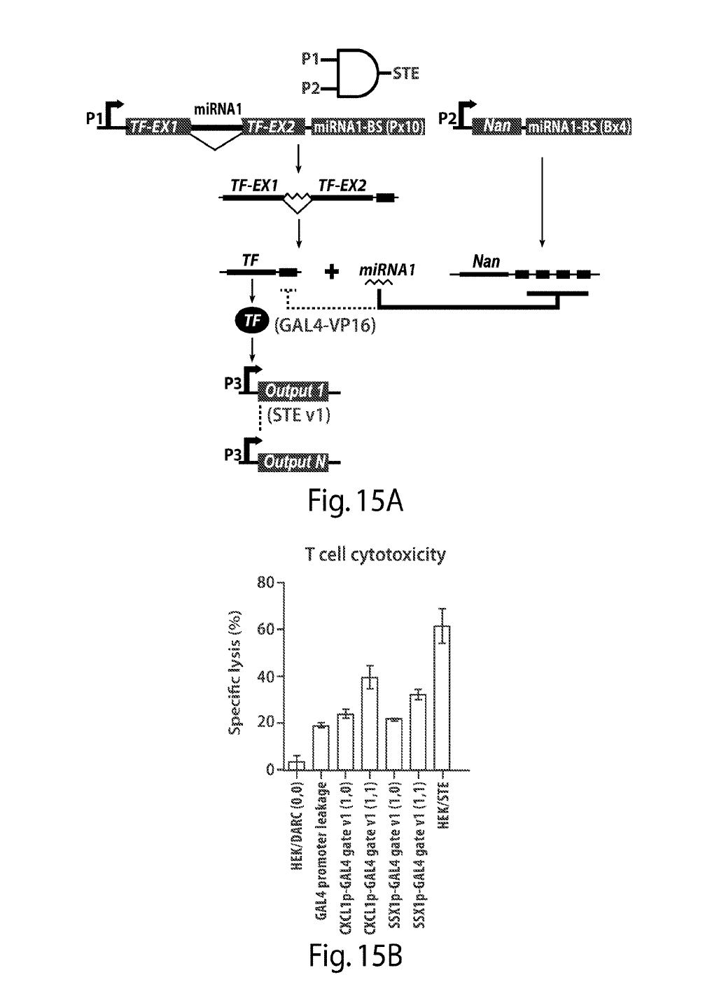

HEK-293T cells were transfected with various DNA constructs (STRICT039, 040) as indicated and Donor #2's T cells were used (FIG. 15). The E:T ratio was 10:1; 6×10⁵:6×10⁴. FIG. 15A shows the circuit used for this T cell cytotoxicity experiment. In FIG. 15B, (1,0) indicated cells transfected with STE only. (1,1) indicated cells transfected with STE and sponge. (0,0) indicated cells transfected with a non-STE protein. 48 hrs post transfection, various HEK-293T cells were harvested and co-cultured with human T cells for 5 hrs. Cytotoxicity by T cells was measured by LDH release assay. Data show that T cells kill STE expressing (1,0) cells (column 3 and 5) and the killing can be greatly enhanced by the AND gate (1,1) architecture (column 4 and 6). T cells exhibit minimal killing on not STE expressing cells (column 1). The killing on (1,0) condition is mainly caused by the leakage of GALA promoter output (column 2 vs. 3 or 5). An additional experiment is conducted to decrease the GAL4 promoter leakage by removing the Kozak sequence of STE v1, making STE v1 output self-degrading by adding miRNA binding sites at 3′ end, and the combination of both mechanisms.

### HEK-293T Cells (GAL4 Gate v2 for STE)

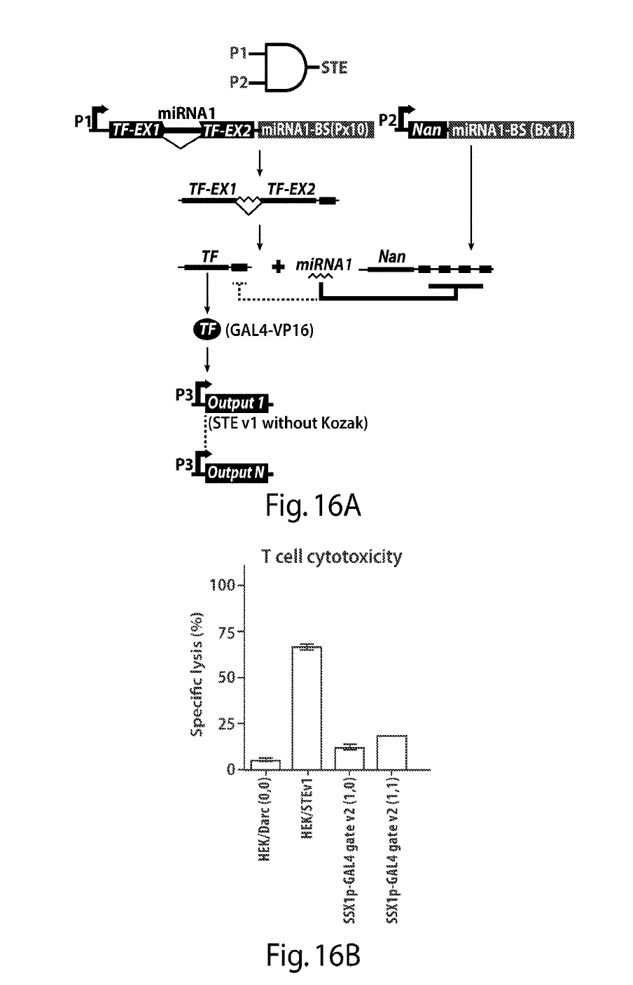

GALA-gate version 2 (v2) architecture can be harnessed to fine tune T cell killing efficiency of tumor cells and exhibit less cytotoxicity at (1,0) state. HEK-293T cells were transfected with various DNA constructs (STRICT039, 040) as indicated and Donor #2's T cells were used (FIG. 16). The E:T ratio was 10:1; 6×10⁵:6×10⁴. FIG. 16A shows the circuit used for this T cell cytotoxicity experiment. In FIG. 16B, (1,0) indicated cells transfected with STE only. (1,1) indicated cells transfected with STE and sponge. (0,0) indicated cells transfected with a non-STE protein. 48 hrs post transfection, various HEK-293T cells were harvested and co-cultured with human T cells for 5 hrs. Cytotoxicity by T cells was measured by LDH release assay (FIG. 16B). Data show that T cells kill STE expressing (1,0) cells (column 3) and the killing can be enhanced by the AND gate (1,1) architecture (column 4). T cells exhibit minimal killing on not STE expressing cells (column 1). The killing on (1,0) state of this version is improved compared to GALA gate v1 architecture (v2 is more closer to basal level (0,0)). An additional experiment is conducted to decrease the killing at (1,0) state. The GAL4 promoter output at (1,0) state is decreased by adding miR binding sites at 3′ end of STE gene.

### HEK-293T Cells (GAL4 Gate v3 for STE)

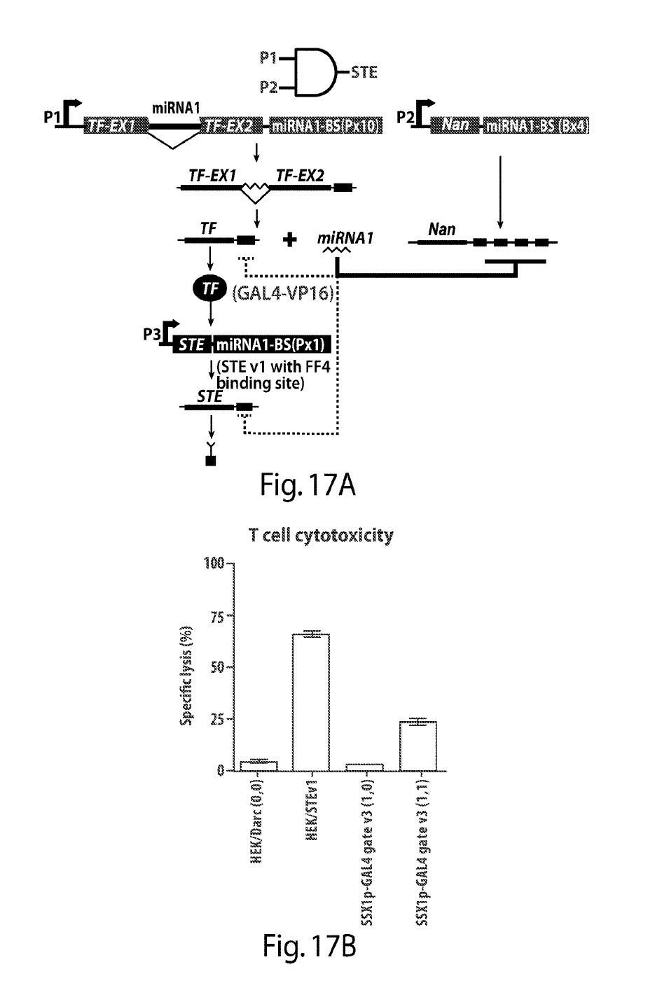

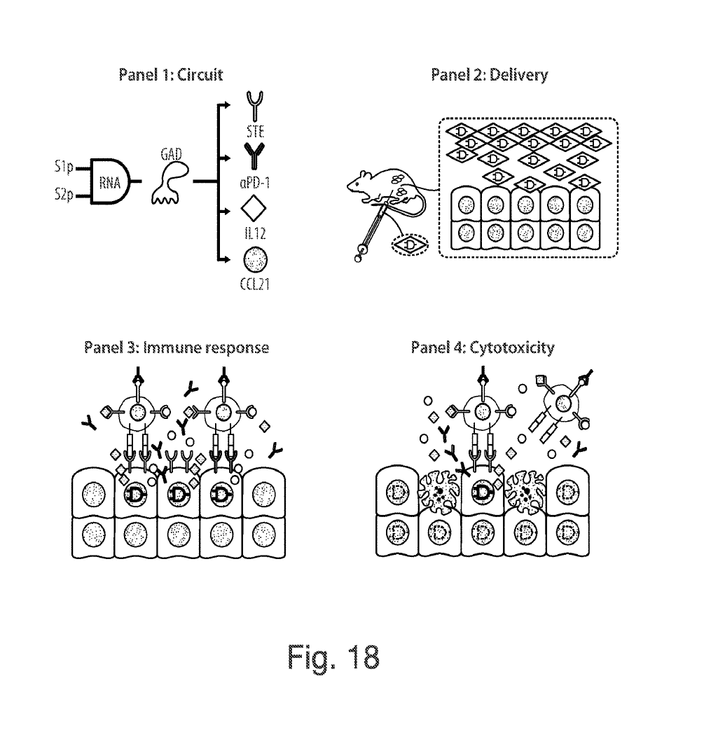

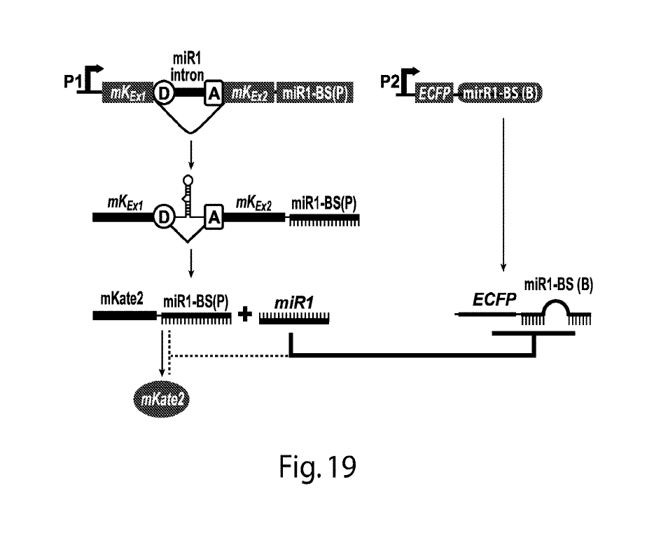

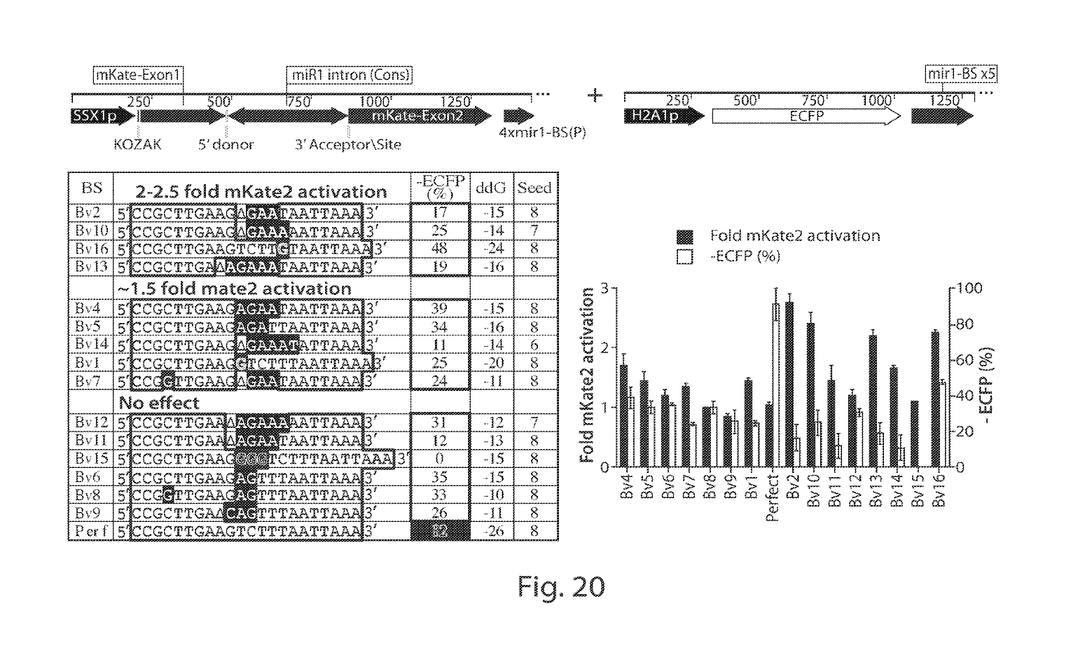

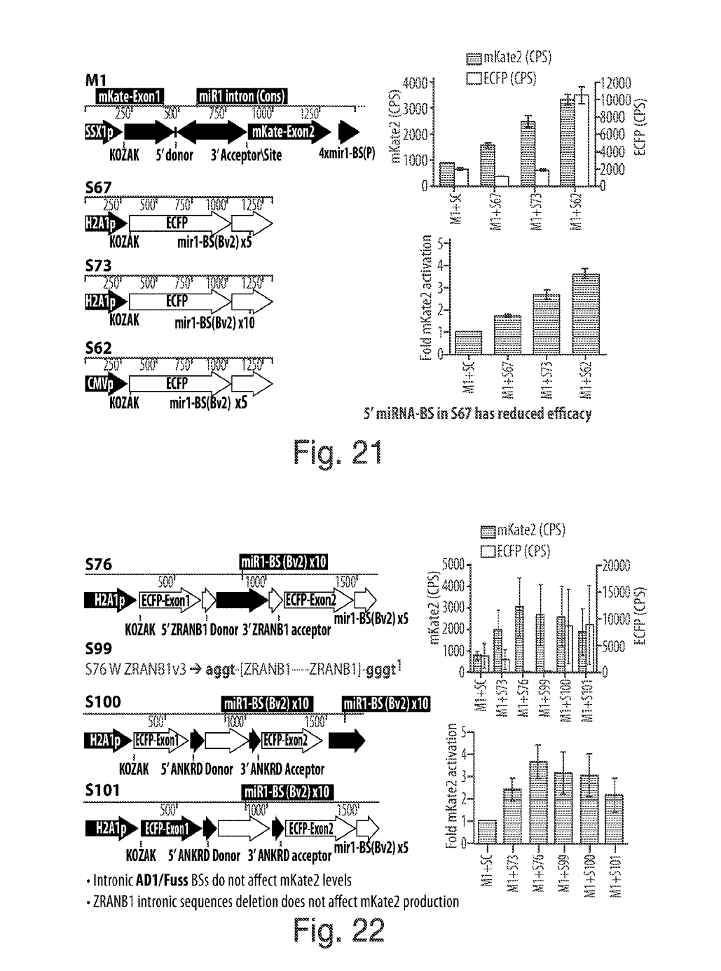

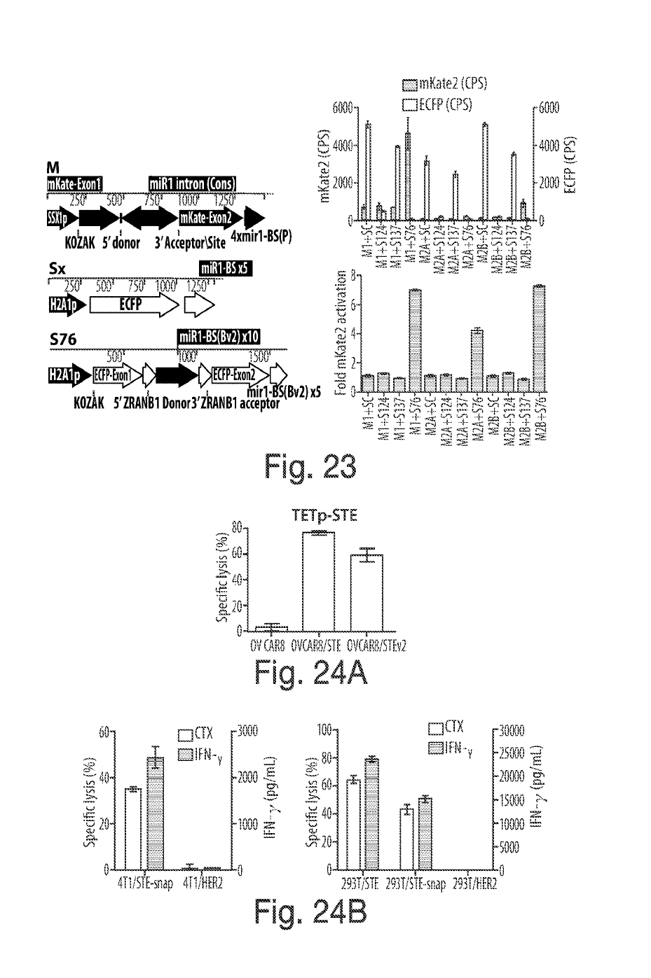

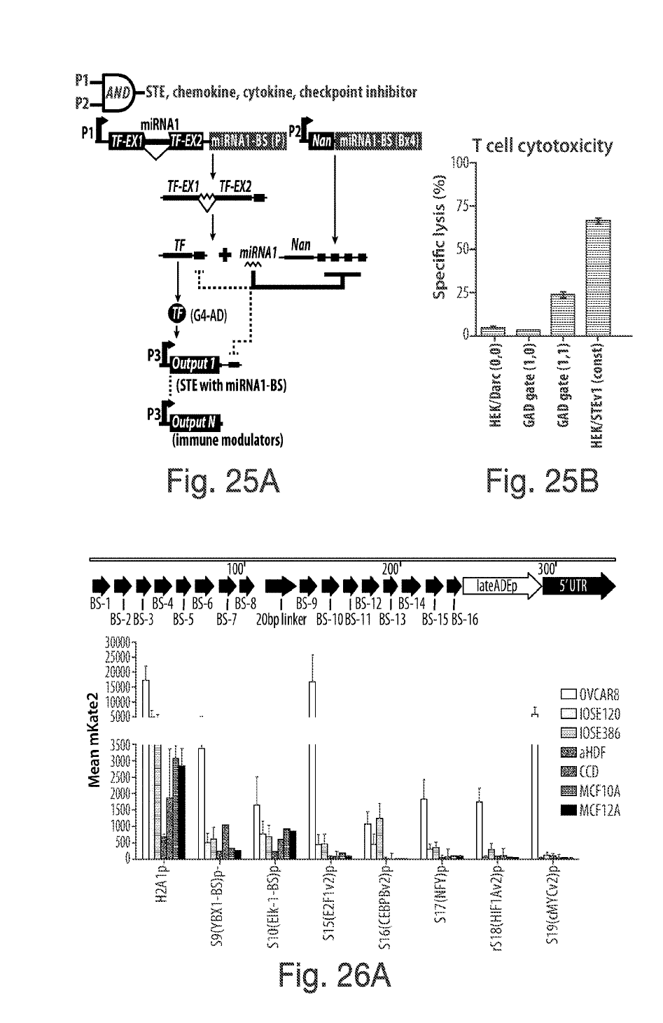

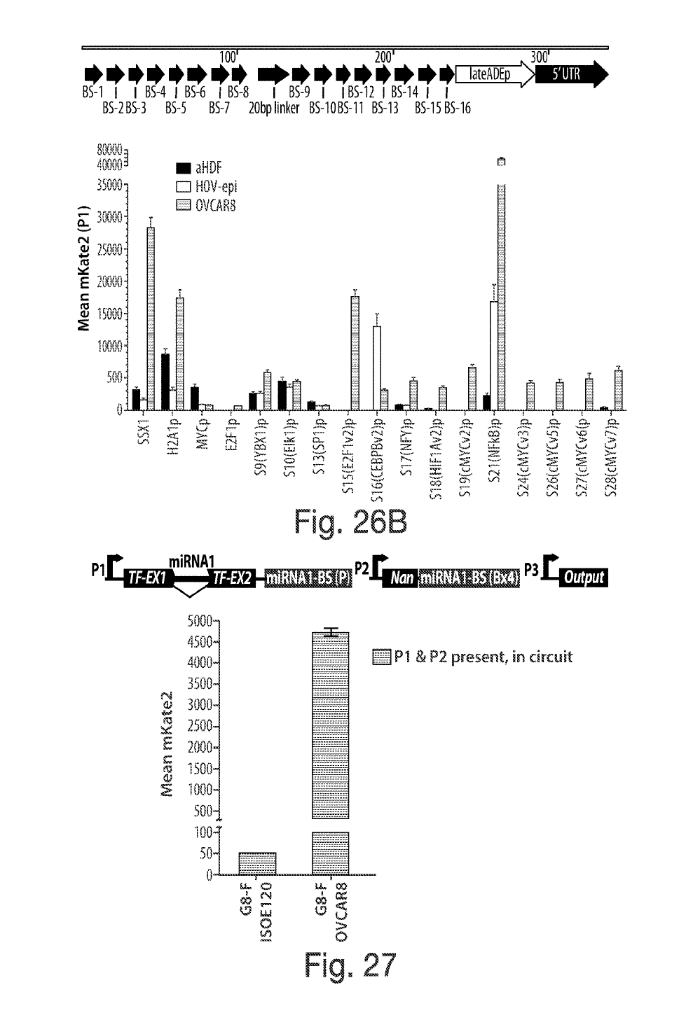

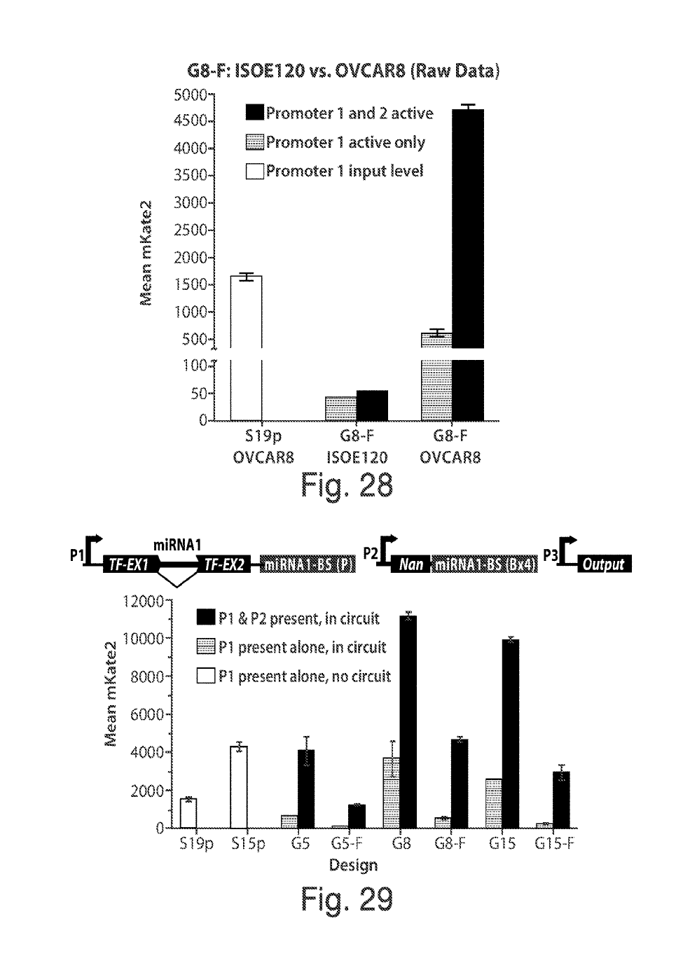

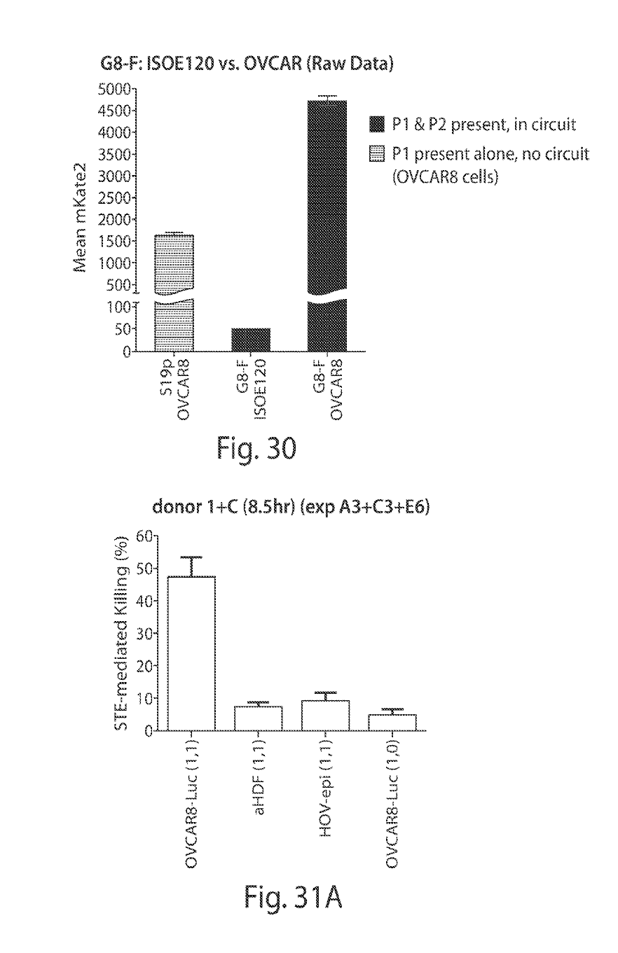

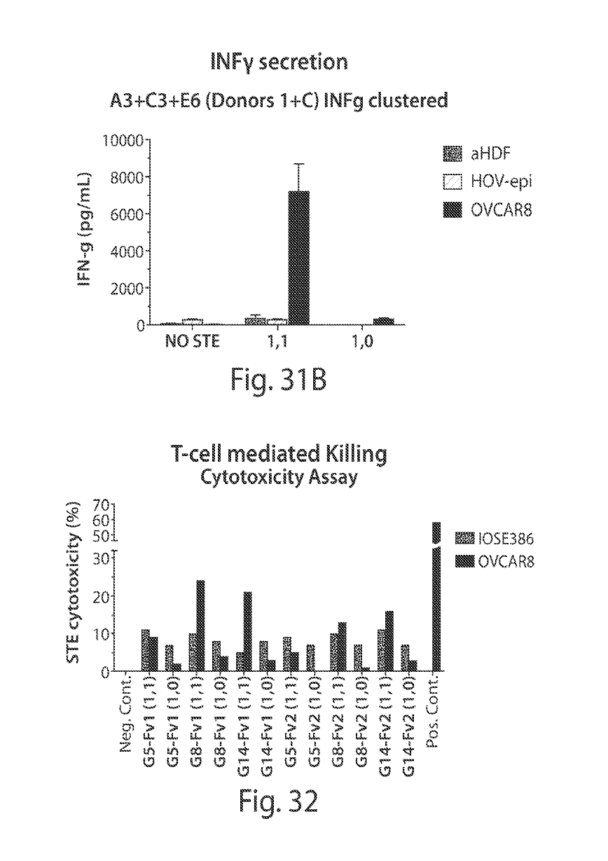

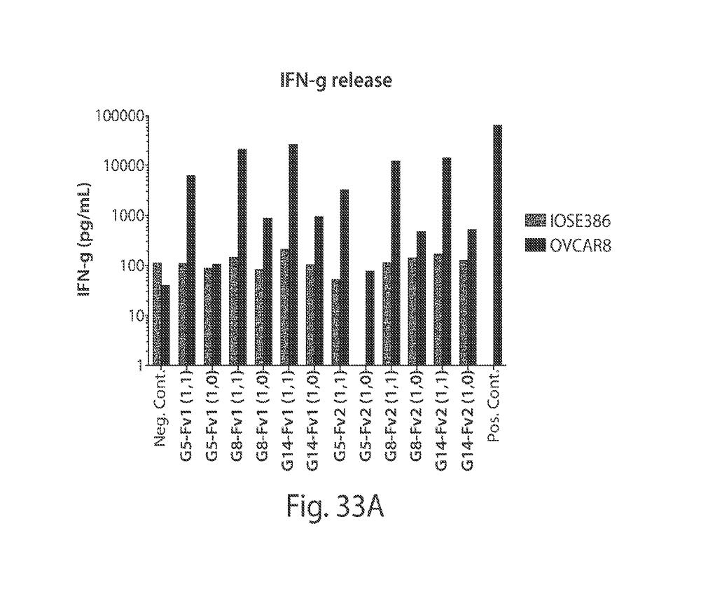

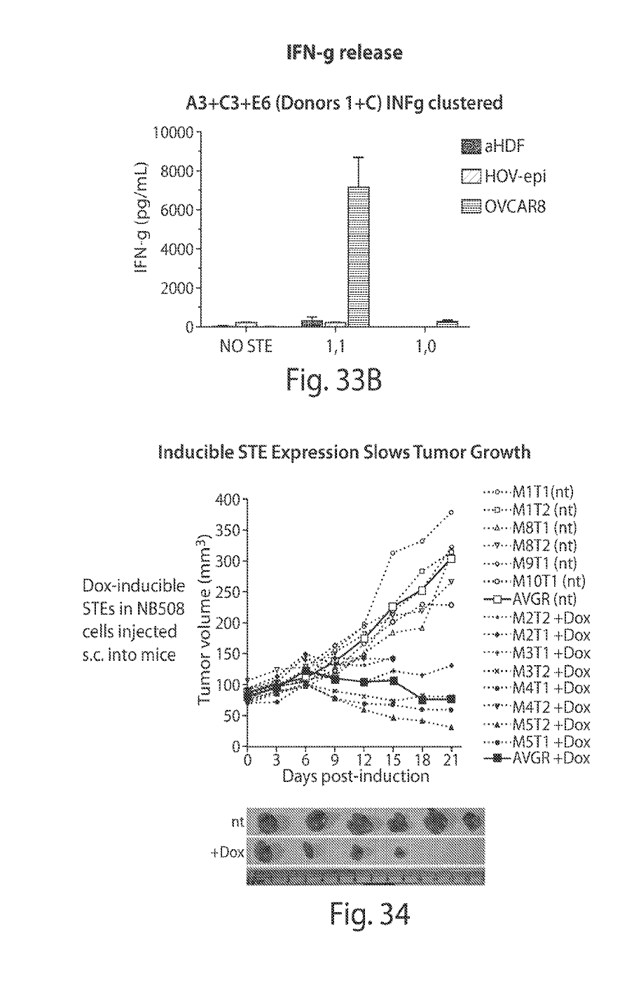

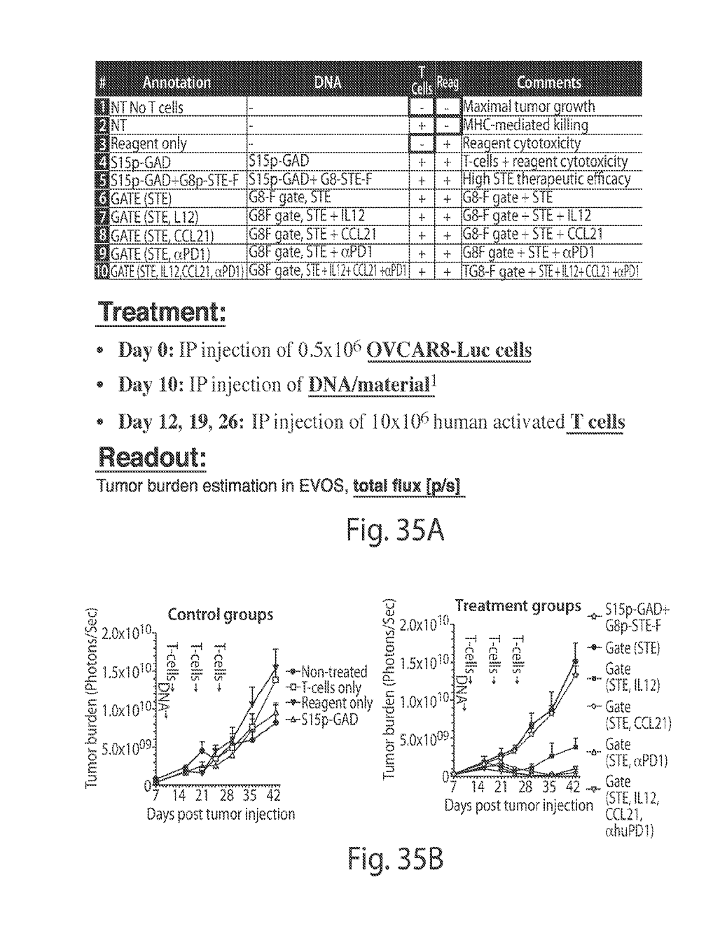

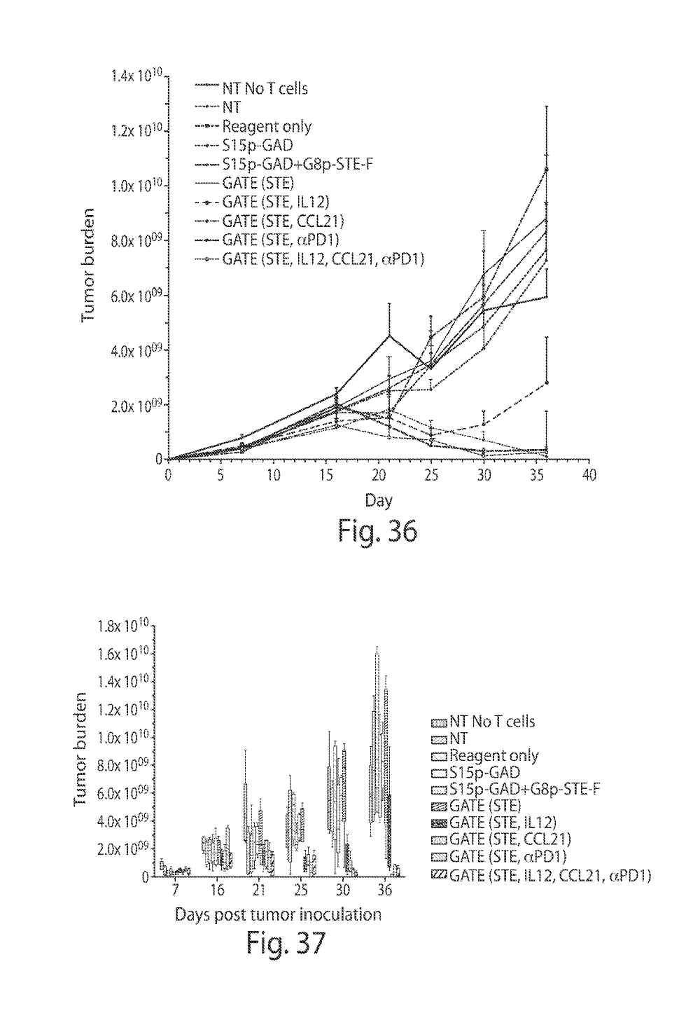

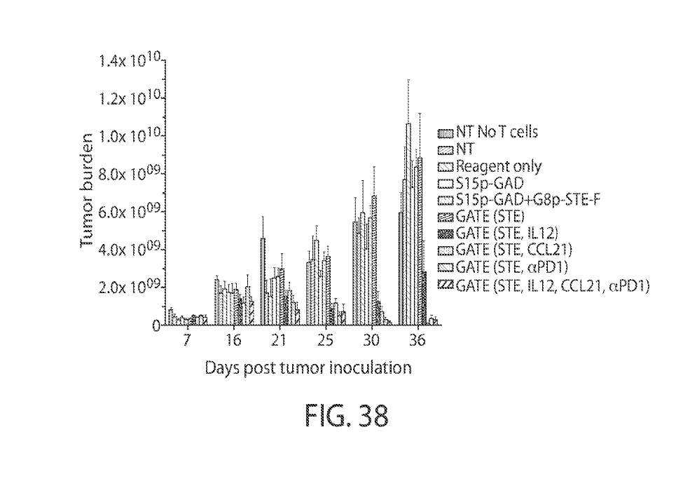

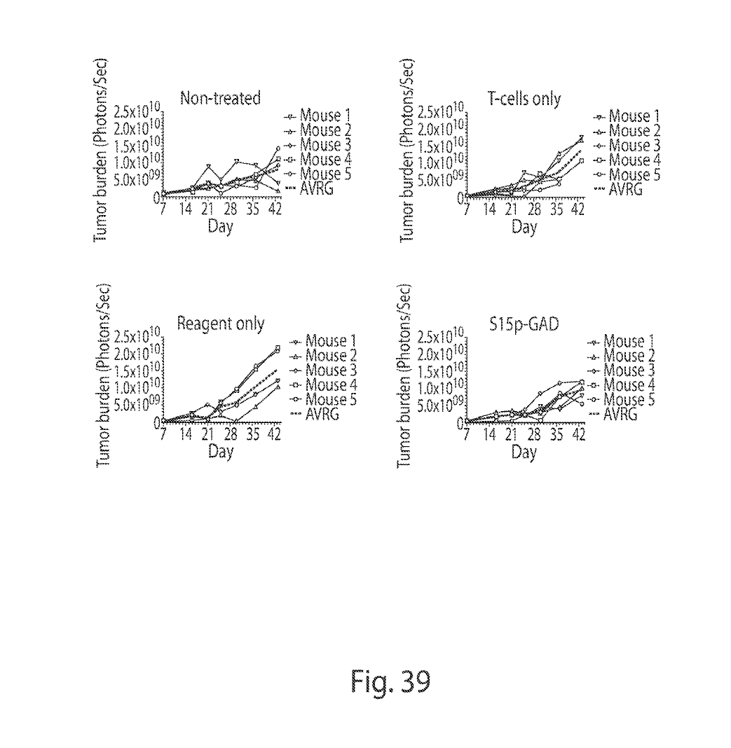

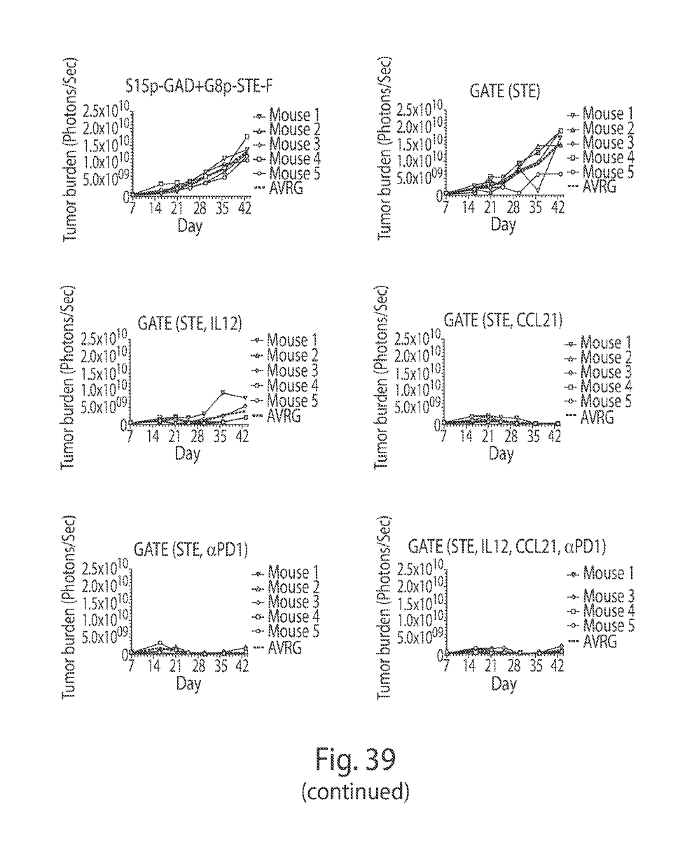

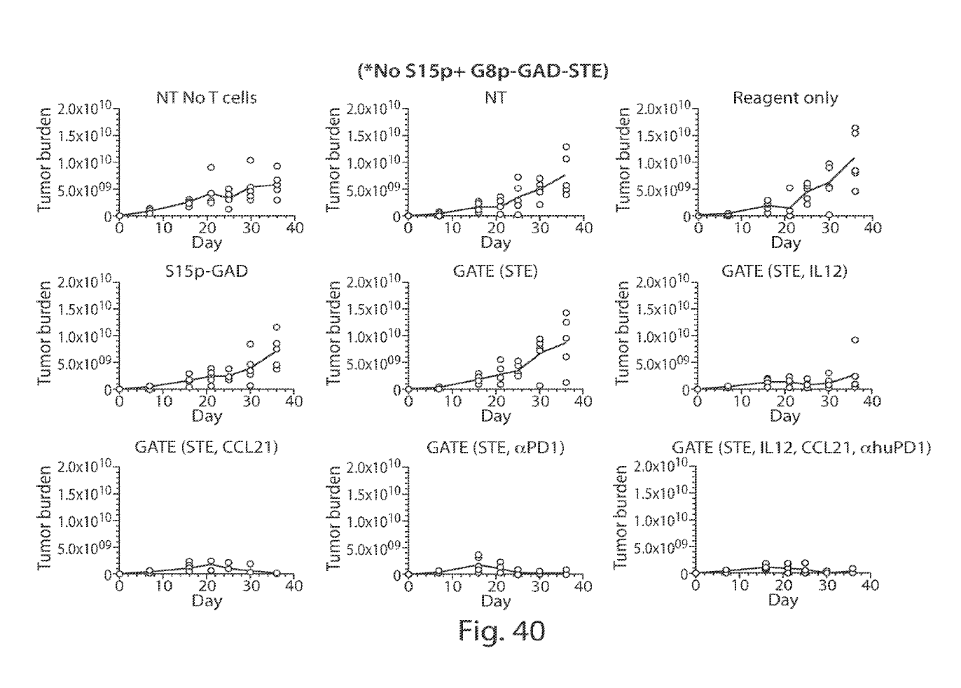

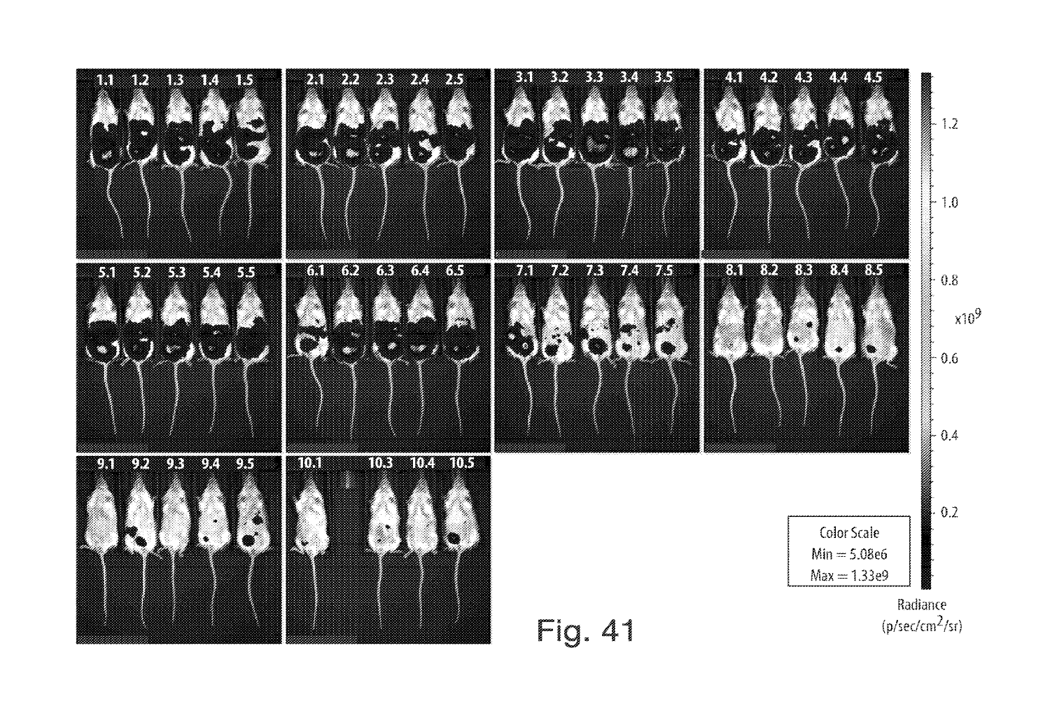

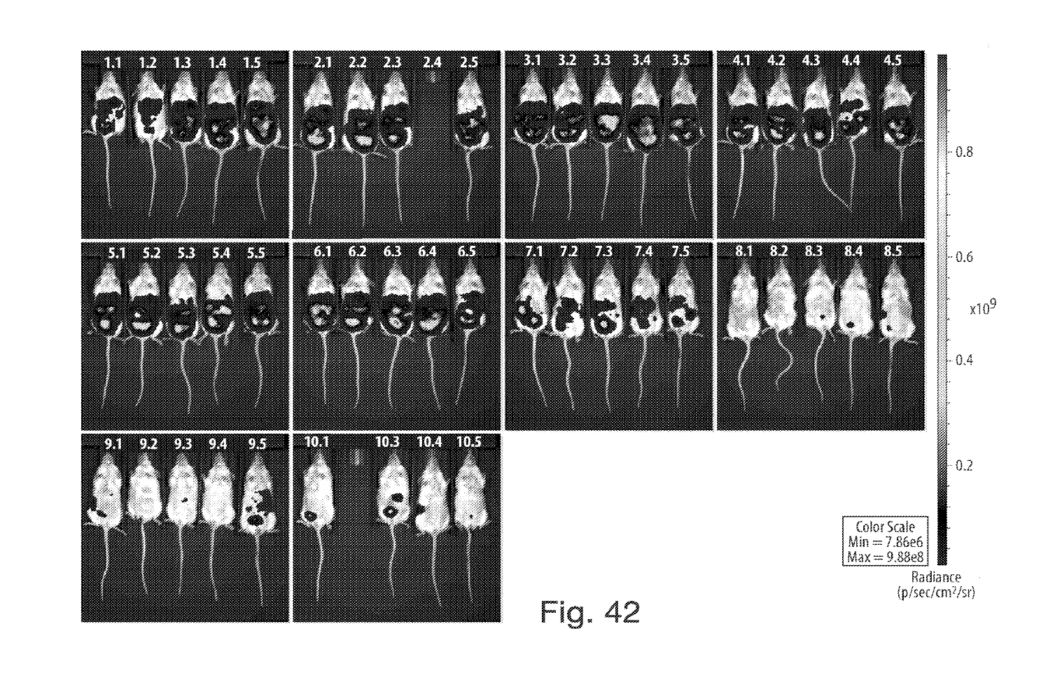

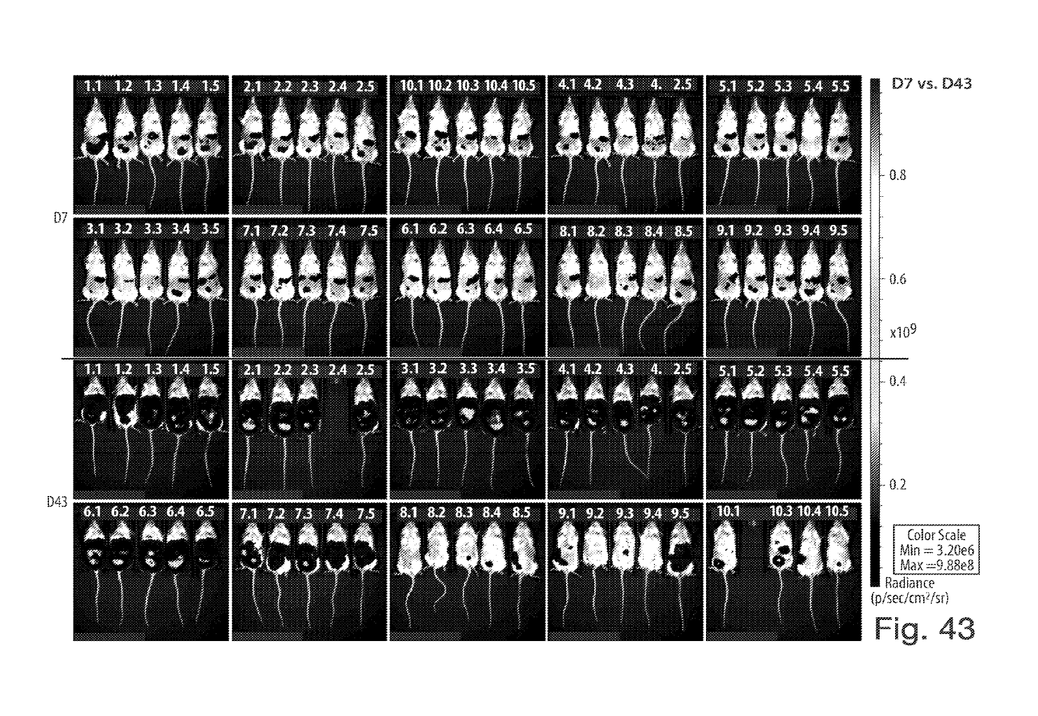

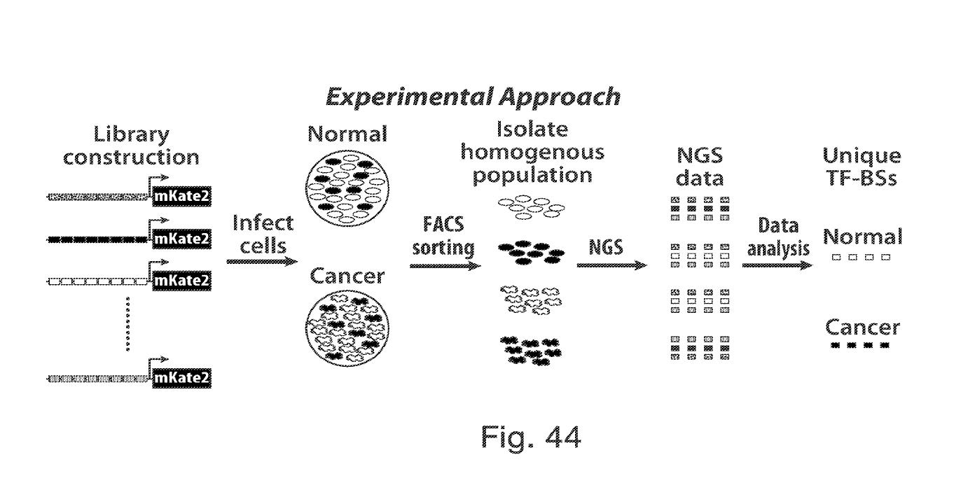

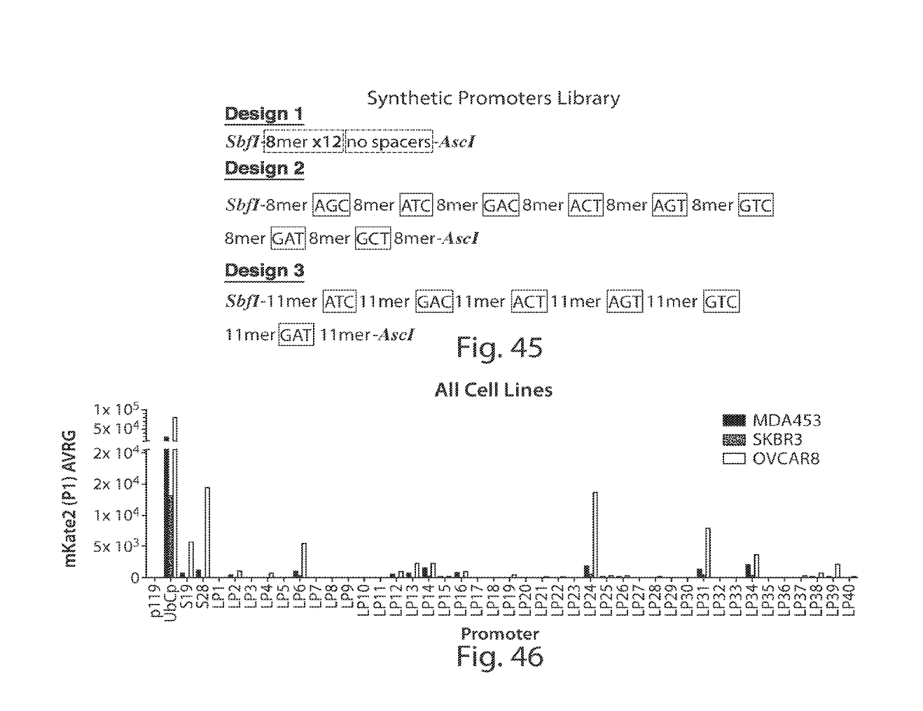

GALA-gate version 3 (v3) architecture can be harnessed to fine tune T cell killing efficiency of tumor cells and exhibit less cytotoxicity at (1,0) state. HEK-293T cells were transfected with various DNA constructs (STRICT039, 040) as indicated and Donor #2's T cells were used (FIG. 16). The E:T ratio was 10:1; 6×10⁵:6×10⁴. FIG. 17A shows the circuit used for this T cell cytotoxicity experiment. In FIG. 17B, (1,0) indicated cells transfected with STE only. (1,1) indicated cells transfected with STE and sponge. (0,0) indicated cells transfected with a non-STE protein. 48 hrs post transfection, various HEK-293T cells were harvested and co-cultured with human T cells for 5 hrs. Cytotoxicity by T cells was measured by LDH release assay (FIG. 17B). Data show that T cells minimally kill STE expressing (1,0) cells (column 3) and only reach efficient killing when the AND gate is active (1,1) (column 4). T cells exhibit minimal killing on not STE expressing cells (column 1). The killing on (1,0) state is as long as (0,0) state. An additional experiment is conducted increase GAL4-VP16 output level or increase GALA binding sites to enhance the killing efficacy of (1,1) state.

### Example 6

This Example addresses two overarching challenges (FIGS. 2A-2B): (1) to create novel breast-cancer therapies that are safe and effective for replacing interventions that have life-threatening toxicities; and (2) to use these new therapies to eliminate the mortality associated with metastatic breast cancer.

Immunotherapy has achieved robust and potentially curative efficacy against cancers in clinical trials Immunotherapies that harness T cell effector functions, such as chimeric antigen receptor (CAR) T cells or bispecific T-cell engagers (BiTEs), can have potent effects [1, 2]. However, there are major challenges associated with these therapies, especially for solid tumors such as breast cancer. Current CAR-T cell therapy requires custom cell isolation, engineering, and expansion for every patient, which is expensive and challenging to scale. Also, CAR-T cells must traffic to tumor sites to mediate killing and require long-term persistence for robust efficacy, which can pose challenges for solid tumors [3].

BiTEs are fusion proteins that include two single-chain variable fragments (scFvs) fused in tandem to enable engagement of tumor cells by T cells, thus resulting in T-cell-triggered tumor killing. BiTE therapy is potent and can confer tumor killing at a concentration five orders-of-magnitude lower than tumor-targeting antibodies (Abs) [2]. However, even multi-bolus injections cannot maintain high serum BiTE concentrations due to their short half lives in vivo (˜2 hours) [4]. Successful BiTE clinical trials treating hematological cancers have all required continuous intravenous infusions for 4 to 8 weeks [2]. Since solid tumors are generally less accessible to immune cells than hematological malignancies, successful BiTE therapy for solid tumors will likely require even longer periods of continuous BiTE infusions, which is undesirable due to potential side effects, patient inconvenience, and reduced efficacy. Finally, both CAR T-cells and BiTE therapies target extracellular tumor-specific antigens that are not available in many cancer types, including triple-negative breast cancer. Furthermore, target antigens can be displayed by normal cells and thus immunotherapy can result in off-target immune responses with severe consequences [5].

In addition to harnessing T cell effector function with CARs or BiTEs, an alternative approach is to deliver genetic circuits into tumor cells that express T-cell-engaging proteins on cancer cell surfaces and activate T-cell-based killing. These Surface T cell Engagers (STEs) can trigger antigen-independent T cell killing of tumor cells in vitro and in vivo [6-10]. However, previous STE studies were not able to build genetic circuits that were only activated in tumor cells. Thus, to avoid systemic toxicity, these constructs were only limited to intra-tumoral injections, resulting in decreased efficacy and the inability to treat systemic diseases [7, 10]. This is a major limitation, because for many cancers and especially breast cancers, metastatic disease is the main reason for mortality. Thus, a scalable therapy that can harness the immune system to treat systemic and metastatic cancers with high anti-tumor specificity is urgently needed, which is provided herein.

Synthetic biologists have developed gene circuits for highly specific intracellular detection of cancer states based on cancer-specific promoters or miRNA profiles [11, 12]. However, further development is required before these tumor-detecting circuits can be used in the clinic. For example, these synthetic tumor-detecting circuits have only been coupled with intracellular killing mechanisms, which restricts their efficacy against tumors because it is virtually impossible to deliver the circuits to 100% of cancer cells. In addition, high targeting specificity is required to avoid damaging healthy tissues. Finally, past circuits have utilized foreign proteins but minimizing ectopic protein expression is essential to avoid inducing host immune responses in normal cells.

To overcome limitations of existing cancer immunotherapies and tumor-detecting gene circuits, provided herein are Tumor Immunotherapy by Gene-circuit Recruited Immunomodulatory Systems (TIGRIS), also referred to as Synthetic Tumor Recruited Immuno-Cellular Therapy (STRICT), a platform technology to trigger potent and effective immunotherapy against tumors from within tumors themselves. TIGRIS is combination of tumor-detecting gene circuits with anti-cancer immunotherapies. Engineered genetic circuits can be delivered to tumors. These engineered genetic circuits are selectively activated only in cancer cells, resulting in the surface display of STEs and the secretion of other immunomodulatory molecules to recruit T cells to target the tumor. We designed tumor-detecting gene circuits with very high specificity to enable TIGRIS therapy to be administered systemically but only be activated locally in cancer cells, resulting in enhanced safety and reduced side effects. Therefore, TIGRIS combines the advantages of systemic delivery (e.g., treating metastasis) with the advantages of localized treatment (e.g., safety, minimal side effects), and enables the benefits below.

We developed TIGRIS against triple-negative breast cancer (TNBC), a difficult-to-treat subset of breast cancer that exhibits aggressive behavior and is correlated with poorer prognosis [13-15]. There are no ideal targeted therapies for TNBC since this subset of breast cancers does not express the estrogen receptor, progesterone receptor, or HER2. TIGRIS should overcome key obstacles associated with other therapies, including:

1) The challenge of breast cancer heterogeneity. Breast cancers are known to be very inter-tumorally and intra-tumorally heterogeneous [16]. For example, HER2 expression heterogeneity is correlated with poor prognosis [17] and traditional targeted therapies cannot cover entire heterogeneous cancer populations. In contrast, we hypothesize that tumor-specific STE expression will first recruit T cells to kill STE-expressing cancer cells. The initial killing should release immunogenic mutant antigens [18] that should recruit additional waves of T cells with a variety of targeting specificities. This would generate a polyclonal immune response against the tumor antigens, cover the broad mutational landscape of the heterogeneous tumor population, and prevent immunoediting-mediated tumor relapse. In addition, almost all targeted therapy can create target-negative tumor variant outgrowth. Since TIGRIS does not require a known tumor-specific antigen to be expressed by tumor cells, it should not be affected by tumor escape mechanisms that involve downregulation of surface antigens.

2) Limited targeting spectra. Unlike CAR-T cell or BiTE therapy, TIGRIS does not depend on the surface expression of tumor-specific antigens that can be hard to identify for many cancers. Rather, TIGRIS is activated by the concerted activity of multiple tumor-specific/tissue-specific promoters via AND gate logic, which results in enhanced specificity versus single promoter systems. These logic circuits can be customized for different promoters and even incorporate tumor-specific/tissue-specific microRNAs for further specificity, thus enabling flexible therapeutic efficacy. Furthermore, these promoters can be identified via tumor cell sequencing and customized for different tumors to overcome immunoedited cancers and heterogeneous cancer cell types.

3) The deadly consequence of metastasis. Metastatic tumor cells are difficult to treat and are responsible for 90% of breast cancer deaths [19]. Our gene circuits can be delivered systemically but only have local effects due to their specificity, thus potentially enabling the detection and destruction of metastases. In addition, we expect that anti-cancer T cells activated by TIGRIS will patrol the body to target metastases for destruction.

4) Evolution of tumor escape variants during targeted therapy. TIGRIS can initiate epitope spreading, and this phenomenon recruits many T cells bearing different tumor-targeting specificities. The probability of tumor escape variants will be much smaller than traditional targeted therapy.

5) The challenge of tumor relapse. Many advanced breast cancers eventually recur and no predictive or preventive measures for relapse are available. Since T cells can differentiate into memory T cells and reside in the body for a long period of time, TIGRIS can prevent future tumor relapse. Here, we provide, as an example, TNBC, a difficult subset of breast cancer to treat using traditional therapies.

6) The challenge of therapeutic delivery. The delivery of discrete therapies, such as nucleic acids or gene circuits using viral or non-viral vectors, is usually unable to target all tumor cells. Since STEs can recruit T cells to initiate tumor killing and initiate epitope-spreading phenomena, this technology can kill surrounding cancer cells as long as the immune response triggered by STE is robust enough, even if our tumor-detecting circuits can only be delivered to a small fraction of tumor cells.

By engineering highly specific cancer-detection circuits to command tumor cells to express STE and other immunomodulators, we can elicit a robust host immune response to eliminate primary tumor cells, target heterogeneous tumors, inhibit local lymph node invasion, and target systemic metastases, while also forming immune memory to protect against future tumor relapse.

### Engineer TIGRIS Constructs and Validate Therapeutic Efficacy In Vitro and In Vivo.

We created novel cancer-detection circuits that command tumor cells to display STEs. We test if STEs can trigger robust immune responses and effectively kill breast cancer cells in vitro and in vivo. The key parameters needed to achieve robust efficacy against solid tumors such as breast cancer with TIGRIS (e.g., the minimal fraction of STE-expressing tumor cells and the minimal STE expression level on tumor cell surfaces) are unknown, so we determine these with in vitro and in vivo assays. We also test whether the TIGRIS-triggered immune response can enable effective anti-tumor therapy despite intratumoral heterogeneity in breast cancers.

Create and validate cancer-detecting circuits that display STEs on tumor cells. We created human and murine STEs (FIG. 12A (top)) by fusing an scFv derived from an anti-human CD3ε Ab (clone: OKT3) or anti-murine CD3ε Ab (clone: 2C11) with inert membrane anchoring proteins (e.g., cytoplasmic truncated Duffy Antigen/Receptor for Chemokines (DARC)), respectively. We performed in vitro T cell cytotoxicity assays and cytokine release assays to test the functionality of the human STE when expressed by various tumor cell lines representing TNBC, chronic myeloid leukemia, and embryonic kidney tumors (4T1, K562, and HEK-293T, respectively). We observed robust cytotoxicity and IFN-γ production by T cells when T cells were co-cultured with STE-expressing tumor cells (FIG. 24B). Since human and murine STEs should only bind to human and murine T cells, respectively, these constructs enable us to confirm that specific T-cell engagement is necessary for therapeutic efficacy.

In addition, we designed synthetic gene circuits to specifically detect intracellular signatures of cancer. We previously engineered cancer-detecting circuits referred to as Dual Promoter Integrators (DPIs) whose output was only expressed when two cancer-specific promoters were activated beyond a minimal threshold, thus implementing an AND gate [12]. The DPI was implemented using non-human transcription factors, which are not ideal for clinical use since they may introduce foreign proteins that could become immunogenic in normal cells. Here, we create an AND gate using RNA only (FIGS. 3A-3D), which have the additional benefit of being more compact than protein-based circuits. This circuit design only expresses an output when two promoters are activated in cancer cells. We constructed and validated the tunability, modularity, and functionality of our RNA-only AND gate architecture using the SSX1 and H2A1 cancer-specific promoters in HEK-293T cells with fluorescent proteins and STEs as outputs (see description below).

We adapt our RNA gates for specifically recognizing breast cancer cells. With our current circuit, there is a ˜2-fold enhancement in T-cell-mediated killing between cells that contain both inputs to the AND gate activated in cancer cells (40% lysis, State 1 in FIG. 4) over cells that contain just one input active in cancer cells (the one that expresses the STE protein only, State 3 in FIGS. 3A-3D). The performance of this circuit (e.g., enhanced ON:OFF ratio) can be further enhanced by increasing the number of miRNA binding sites in the STE transcript, modifying the miRNA backbone for more robust miRNA production, producing multiple miRNA copies per STE transcript, testing libraries of different miRNAs and sponges, modifying sponge sequences and architectures, minimizing leakiness with mRNA degradation tags, implementing trans-cleaving ribozymes for the removal of the miRNA-binding sites in the STE transcript, and including additional miRNA binding sites in the STE transcript that are bound and repressed by endogenous miRNAs that are highly expressed in normal cells but downregulated in tumor cells [31].

We also test other cancer-specific and tissue-specific promoters (e.g., RPC1 and RRM2 that are highly breast cancer specific and have been validated in TNBC cell lines [32]) and validate that our circuit is activated in 4T1 cancer cells but not in normal cells (e.g., COMMA-1D, EpH4, MCF10A).

We tested circuit functionality by transfecting or stably integrating the circuits into tumor cells. We further encode our circuits in adenoviral, AAV, or HSV vectors in order to enable delivery into 4T1 and normal breast cell lines to verify tumor detection sensitivity, specificity, and tunability. We also leverage oncolytic HSV vectors, such as T-VEC, which have been used for cancer therapy in human patients [33].

If some of the cancer-specific promoters described above, in some instances, do not achieve specific activation in 4T1 cells, additional cancer-specific promoters may be identified with comparative transcriptomics and by screening barcoded promoter libraries for specific activation in target cells using FACS and sequencing. If some RNA-only circuits do not achieve significant ON:OFF ratios, human transcription factors (such as artificial zinc-finger proteins [27]) may be used to minimize the introduction of potentially immunogenic foreign proteins.

Identify the minimal percentage of tumor cells that need to be targeted by TIGRIS for in vivo efficacy. We elucidate the minimal percentage of tumor cells that need to be targeted by our gene circuits to achieve robust therapeutic efficacy in vivo. This information is used for designing systemic delivery strategies, since these are unlikely, in some instances, to target 100% of tumor cells. We mix STE-displaying tumor cells (4T1/STE+) with non-STE-displaying counterparts (4T1/STE-) at various ratios and directly implant them into immune-competent BALB/c mice mammary pads to create orthotopic breast cancer models. The 4T1 murine model resembles advanced human TNBC and is highly malignant and metastatic [34, 35]. Tumor growth kinetics will be monitored by measuring tumor volume with calipers every other day. We monitor animal survival over time with experiments that will be kept running for at least two times longer than the mean survival time of control mice. The minimal percentage of STE-expressing tumor cells needed to efficiently inhibit the growth of injected tumor cells will be identified. Tumor cell lines expressing human STEs are used as controls to validate T-cell-engagement specificity. We utilize 4-6 mice per experimental condition.

When there are sufficient STE-expressing cells, tumor growth should be partially or totally suppressed, resulting in surviving mice that are disease free over long time periods. We use Student's t-test and one-way ANOVA to compare tumor volumes between 2 groups and between >2 groups, respectively. To analyze survival experiments, we use Kaplan-Meier survival analysis. We also adoptively transfer T cells engineered with a dual bioluminescent reporter system to track the dynamics of T-cell tumor infiltration and activation with in vivo imaging [36]. We extend this work with C3(1)/SV40 T-antigen transgenic mice [37], a very aggressive spontaneous TNBC model, to verify our findings in a more physiologically relevant tumor model.

We determine the lower limit of tumor cells that need to express STEs to confer robust in vivo efficacy. For limits greater than the average gene delivery efficiency, we design new circuits that can simultaneously secrete multiple immunostimulatory effectors. These molecules include chemokines that actively attract T cells (e.g., CCL19 and CCL21) [38], cytokines that are immunostimulatory and can condition tumor microenvironments (e.g., IL-12, IL-15, and IL-21) [39], and immune-checkpoint blockade Abs (e.g., anti-CTLA4 or anti-PD1 Abs) that can unleash brakes in T cell activity [40]. This combinatorial approach should enhance therapeutic efficacy against heterogeneous breast cancers. For example, anti-PD1 Abs have achieved response rates of 20-50% in multiple clinical trials targeting various solid tumor types. However, pre-existing immunity is required for patients to respond to anti-PD1 Abs [41, 42]. By expressing STEs and anti-PD1 Abs together, STEs can help create pre-existing immunity against tumor-associated and mutated antigens while anti-PD1 Abs can enhance T-cell function, proliferation, and infiltration into tumors, especially those that express PD-L1 (PD-1 ligand) to shut down T-cell function [43, 44].

### Evaluate TIGRIS Against Metastatic Cancer and Relapse.

In advanced breast cancer, tumor cell lymph node infiltration and systemic metastasis is commonly observed and is responsible for 90% of breast cancer mortality. The standard of care after surgery is chemotherapy combined with targeted therapy, but this is not very effective for TNBCs [13-15]. In addition, 20-30% of patients diagnosed with invasive breast cancer will relapse after therapy but there are no preventive measures or diagnostic markers for early detection of recurrence. We test whether immune cells triggered by TIGRIS can eliminate lymph node and systemic metastasis, and establish long-term immune memory. TIGRIS may obviate the need for systemic chemotherapy and surgical removal of lymph nodes, which is the most common cause of morbidity, and provide protection against tumor relapse.

Determine if TIGRIS can eliminate primary tumors and metastases via systemic delivery. We test if systemic viral delivery of the engineered genetic circuits can eliminate primary and metastatic tumors in vivo. We engineer 4T1 cells to express luciferase for in vivo imaging. To test for efficacy against metastases, we use the 4T1 orthotopic model from above but only initiate our virally delivered circuit therapy when metastases in lymph nodes and vital organs (expected in lung, liver, bone, and brain) are observed. We monitor the overall tumor burden (primary+metastatic tumors) in the mouse models.

We test different treatment protocols by varying parameters such as viral vector concentration, timing, and types [45]. We track the in vivo immune response generated by TIGRIS via live animal imaging. We should see reductions in tumor growth in primary and metastatic tumors after treatment, especially in organs that immune cells can readily enter, such as lung, liver, and bone. Reduction in brain metastases may also be possible since T-cell-based immunotherapy has been shown to infiltrate the cerebral spinal fluid [1]. We compare TIGRIS versus known chemotherapy regimens, such as taxane and anthracycline [46]

If, in some instances, primary tumors are not eliminated with STE expression alone, we augment the therapy with multiple immunostimulatory effectors described above. We also test whether multiple viral injections can enhance therapeutic efficacy. In addition, we surgically remove the primary tumor before and after circuit therapy to mimic common clinical practice and to test how surgical removal of primary tumors may affect the immune response against metastases.

Systemic circuit delivery may, in some instances, pose a challenge for achieving high therapeutic efficacy. We improve viral delivery, in some embodiments, by pseudotyping our vectors (e.g., adenovirus) with small peptides to target other cell surface receptors [47]. In some embodiments, we adapt oncolytic viruses that have been shown to target breast cancers to take advantage of simultaneous tumor lysis and immunotherapy [48]. In some instances, viral particles may only penetrate the tumor periphery in many solid tumors. Thus, we can express iRGD tumor penetrating peptides as additional circuit outputs [49]. These peptides can significantly enhance the tumor penetration of many therapeutic agents, including Abs, oncolytic viruses, and nanoparticles [49-51].

In addition to testing systemic delivery, we also determine the therapeutic efficacy of localized circuit delivery into primary tumor cells for treating systemic metastases. A localized tumor injection of the immunomodulatory oncolytic virus, T-Vec, can cause shrinkage of uninjected tumors [33]. This finding indicates that localized delivery of TIGRIS circuits, which can be achieved with viral or non-viral vectors, may also confer therapeutic efficacy. By generating a local immune response in injected tumors, TIGRIS may initiate a systemic immune response that could target metastatic tumors.

STE expression should be terminated when all gene-circuit-containing tumor cells are killed. However, to enhance controllability and safety, in some embodiments, we build synthetic safety mechanisms into our gene circuits. In these designs, if the gate is operating properly in normal cells, it should be OFF and should not express any foreign proteins. Thus, only if the gate malfunctions in normal cells or if the gate operates properly in cancer cells would the therapeutic output proteins be expressed along with safety mechanisms that can be externally toggled. First, we engineer inducible circuits to terminate STE expression and/or kill STE-expressing cells. Specifically, the STE output is replaced with a synthetic transcription factor, such as GAL4BD-VP16AD (GAD). In this architecture, genes for the STE, immunostimulatory molecules, and iRGD peptides, together with the conditional killer gene TK1, are regulated by the GAD-responsive promoter, G5p. Thus, foreign proteins are expressed, along with STEs, TK1, and other output genes, only when the logic gate is active. Addition of the TK1 substrate (e.g., ganciclovir or acyclovir) enables the killing of cells in which a circuit is active. Alternatively, we generate inducible transcription factors as outputs of our logic gates (e.g., the doxycycline-responsive transcription factor rtTA3), instead of GAD, to drive therapeutic output expression. In this case, the whole system would not be activated without the administration of exogenous inducers (e.g., doxycycline), thus providing a simple and safe mechanism to control treatment initiation and termination with FDA-approved small molecules. As a final layer of safety, we implement inducible expression of secreted STE antagonists, such as CD3ε on its own, that can titrate out functional STEs.

Test if TIGRIS can elicit immune memory to protect against future tumor relapse. TIGRIS should initiate long-term immune memory against recurrent breast cancer. To show this, we re-challenge long-term survivors (from above) with 4T1 tumor cells via tail vein injection. Tail vein injection of 4T1 tumor cells mainly results in lung metastases, which is a common metastatic site for breast cancers [52]. Live animal imaging is performed to monitor tumor seeding in the lung and other vital organs to determine if there is protective immunity against re-introduced tumor cells.

If, in some instances, initial treatment elicits very robust responses against primary tumors but no significant protection against re-challenges, we design the tumor-detecting circuits to additionally secrete IL-7 and IL-15, since these drive memory T cell formation [53]. Furthermore, the 4T1 tumor model is very immunosuppressive [54]. Thus, incorporating checkpoint-blockade Abs and/or pro-inflammatory cytokines (see above), in some instances, may help to generate a more robust memory response.

### REFERENCES FOR EXAMPLE 6

1. Maude, S. L., et al., Chimeric antigen receptor T cells for sustained remissions in leukemia. N Engl J Med, 2014. 371(16): p. 1507-17.
2. Bargou, R., et al., Tumor regression in cancer patients by very low doses of a T cell-engaging antibody. Science, 2008. 321(5891): p. 974-7.
3. Gilham, D. E., et al., CAR-T cells and solid tumors: tuning T cells to challenge an inveterate foe. Trends Mol Med, 2012. 18(7): p. 377-84.
4. Klinger, M., et al., Immunopharmacologic response of patients with B-lineage acute lymphoblastic leukemia to continuous infusion of T cell-engaging CD19/CD3-bispecific BiTE antibody blinatumomab. Blood, 2012. 119(26): p. 6226-33.
5. Kershaw, M. H., et al., Clinical application of genetically modified T cells in cancer therapy. Clin Trans Immunol, 2014. 3: p. e16.
6. Liao, K. W., Y. C. Lo, and S. R. Roffler, Activation of lymphocytes by anti-CD3 single-chain antibody dimers expressed on the plasma membrane of tumor cells. Gene Ther, 2000. 7(4): p. 339-47.
7. Paul, S., et al., Tumor gene therapy by MVA-mediated expression of T-cell-stimulating antibodies. Cancer Gene Ther, 2002. 9(5): p. 470-7.
8. Liao, K. W., et al., Stable expression of chimeric anti-CD3 receptors on mammalian cells for stimulation of antitumor immunity. Cancer Gene Ther, 2003. 10(10): p. 779-90.
9. Yang, Z. M., et al., Anti-CD3 scFv-B7.1 fusion protein expressed on the surface of HeLa cells provokes potent T-lymphocyte activation and cytotoxicity. Biochem Cell Biol, 2007. 85(2): p. 196-202.
10. Liu, Y., et al., Adenovirus-mediated intratumoral expression of immunostimulatory proteins in combination with systemic Treg inactivation induces tumor-destructive immune responses in mouse models. Cancer Gene Ther, 2011. 18(6): p. 407-18.
11. Weber, W. and M. Fussenegger, Emerging biomedical applications of synthetic biology. Nat Rev Genet, 2012. 13(1): p. 21-35.
12. Nissim, L. and R. H. Bar-Ziv, A tunable dual-promoter integrator for targeting of cancer cells. Mol Syst Biol, 2010. 6: p. 444.
13. Hudis, C. A. and L. Gianni, Triple-negative breast cancer: an unmet medical need. Oncologist, 2011. 16 Suppl 1: p. 1-11.
14. Andre, F. and C. C. Zielinski, Optimal strategies for the treatment of metastatic triple-negative breast cancer with currently approved agents. Ann Oncol, 2012.23 Suppl 6: p. vi46-51.
15. Foulkes, W. D., I. E. Smith, and J. S. Reis-Filho, Triple-negative breast cancer. N Engl J Med, 2010. 363(20): p. 1938-48.
16. Polyak, K., Heterogeneity in breast cancer. J Clin Invest, 2011. 121(10): p. 3786-8.
17. Seol, H., et al., Intratumoral heterogeneity of HER2 gene amplification in breast cancer: its clinicopathological significance. Mod Pathol, 2012. 25(7): p. 938-48.
18. Lawrence, M. S., et al., Discovery and saturation analysis of cancer genes across 21 tumour types. Nature, 2014. 505(7484): p. 495-501.
19. Weigelt, B., J. L. Peterse, and L. J. van 't Veer, Breast cancer metastasis: markers and models. Nat Rev Cancer, 2005. 5(8): p. 591-602.
20. Yang, L., et al., Permanent genetic memory with >1-byte capacity. Nat Methods, 2014.
21. Nissim, L., et al., Multiplexed and Programmable Regulation of Gene Networks with an Integrated RNA and CRISPR/Cas Toolkit in Human Cells. Molecular Cell, 2014. 54(4): p. 698-710.
22. Farzadfard, F. and T. K. Lu, Genomically encoded analog memory with precise in vivo DNA writing in living cell populations. Science, 2014.
23. Siuti, P., J. Yazbek, and T. K. Lu, Synthetic circuits integrating logic and memory in living cells. Nat Biotechnol, 2013. 31(5): p. 448-52.
24. Purcell, O., J. Peccoud, and T. K. Lu, Rule-Based Design of Synthetic Transcription Factors in Eukaryotes. ACS Synthetic Biology, 2013.
25. Farzadfard, F., S. D. Perli, and T. K. Lu, Tunable and Multi-Functional Eukaryotic Transcription Factors Based on CRISPR/Cas. ACS Synthetic Biology, 2013.
26. Daniel, R., et al., Synthetic analog computation in living cells. Nature, 2013. 497(7451): p. 619-23.
27. Khalil, A., et al., A Synthetic Biology Framework for Programming Eukaryotic Transcription Functions. Cell, 2012. 150(3): p. 647-658.
28. Cheng, A. A. and T. K. Lu, Synthetic Biology: An Emerging Engineering Discipline. Annual Review of Biomedical Engineering, 2012. 14(1): p. 155-178.
29. Lu, T. K., A. S. Khalil, and J. J. Collins, Next-generation synthetic gene networks. Nat Biotechnol, 2009. 27(12): p. 1139-50.
30. Friedland, A. E., et al., Synthetic gene networks that count. Science, 2009. 324(5931): p. 1199-202.
31. Boyerinas, B., et al., The role of let-7 in cell differentiation and cancer. Endocr Relat Cancer, 2010. 17(1): p. F19-36.
32. Yun, H. J., et al., Transcriptional targeting of gene expression in breast cancer by the promoters of protein regulator of cytokinesis 1 and ribonuclease reductase 2. Exp Mol Med, 2008. 40(3): p. 345-53.
33. Senzer, N. N., et al., Phase II clinical trial of a granulocyte-macrophage colony-stimulating factor-encoding, second-generation oncolytic herpesvirus in patients with unresectable metastatic melanoma. J Clin Oncol, 2009. 27(34): p. 5763-71.
34. Fantozzi, A. and G. Christofori, Mouse models of breast cancer metastasis. Breast Cancer Res, 2006. 8(4): p. 212.
35. Kaur, P., et al., A mouse model for triple-negative breast cancer tumor-initiating cells (TNBC-TICs) exhibits similar aggressive phenotype to the human disease. BMC Cancer, 2012. 12: p. 120.
36. Na, I. K., et al., Concurrent visualization of trafficking, expansion, and activation of T lymphocytes and T-cell precursors in vivo. Blood, 2010. 116(11): p. e18-25.
37. Green, J. E., et al., The C3(1)/SV40 T-antigen transgenic mouse model of mammary cancer: ductal epithelial cell targeting with multistage progression to carcinoma. Oncogene, 2000. 19(8): p. 1020-7.
38. Nguyen-Hoai, T., et al., CCL21 (SLC) improves tumor protection by a DNA vaccine in a Her2/neu mouse tumor model. Cancer Gene Ther, 2012. 19(1): p. 69-76.
39. Ferrone, C. R., et al., Adjuvanticity of plasmid DNA encoding cytokines fused to immunoglobulin Fc domains. Clin Cancer Res, 2006. 12(18): p. 5511-9.
40. Hirano, F., et al., Blockade of B7-H1 and PD-1 by monoclonal antibodies potentiates cancer therapeutic immunity. Cancer Res, 2005. 65(3): p. 1089-96.
41. Tumeh, P. C., et al., PD-1 blockade induces responses by inhibiting adaptive immune resistance. Nature, 2014. 515(7528): p. 568-71.
42. Gubin, M. M., et al., Checkpoint blockade cancer immunotherapy targets tumour-specific mutant antigens. Nature, 2014. 515(7528): p. 577-81.
43. Ghebeh, H., et al., The B7-H1 (PD-L1) T lymphocyte-inhibitory molecule is expressed in breast cancer patients with infiltrating ductal carcinoma: correlation with important high-risk prognostic factors. Neoplasia, 2006. 8(3): p. 190-8.
44. Soliman, H., F. Khalil, and S. Antonia, PD-L1 expression is increased in a subset of basal type breast cancer cells. PLoS One, 2014. 9(2): p. e88557.
45. Magnusson, M. K., et al., A transductionally retargeted adenoviral vector for virotherapy of Her2/neu-expressing prostate cancer. Hum Gene Ther, 2012. 23(1): p. 70-82.
46. Nabholtz, J. M. and A. Riva, Taxane/anthracycline combinations: setting a new standard in breast cancer? Oncologist, 2001. 6 Suppl 3: p. 5-12.
47. Dmitriev, I., et al., An adenovirus vector with genetically modified fibers demonstrates expanded tropism via utilization of a coxsackievirus and adenovirus receptor-independent cell entry mechanism. J Virol, 1998. 72(12): p. 9706-13.
48. Zhang, Z., et al., Intravenous administration of adenoviruses targeting transforming growth factor beta signaling inhibits established bone metastases in 4T1 mouse mammary tumor model in an immunocompetent syngeneic host. Cancer Gene Ther, 2012. 19(9): p. 630-6.
49. Sugahara, K. N., et al., Coadministration of a tumor-penetrating peptide enhances the efficacy of cancer drugs. Science, 2010. 328(5981): p. 1031-5.
50. Hamilton, A. M., et al., Nanoparticles coated with the tumor-penetrating peptide iRGD reduce experimental breast cancer metastasis in the brain. J Mol Med (Berl), 2015.
51. Puig-Saus, C., et al., iRGD tumor-penetrating peptide-modified oncolytic adenovirus shows enhanced tumor transduction, intratumoral dissemination and antitumor efficacy. Gene Ther, 2014. 21(8): p. 767-74.
52. Tao, K., et al., Imagable 4T1 model for the study of late stage breast cancer. BMC Cancer, 2008. 8: p. 228.
53. Cieri, N., et al., IL-7 and IL-15 instruct the generation of human memory stem T cells from naive precursors. Blood, 2013. 121(4): p. 573-84.
54. Chen, L., et al., Rejection of metastatic 4T1 breast cancer by attenuation of Treg cells in combination with immune stimulation. Mol Ther, 2007. 15(12): p. 2194-202.

### Example 7

Synthetic Tumor Recruited Immuno-Cellular Therapy for Ovarian Cancer. New therapeutic strategies are needed to treat primary and metastatic ovarian cancer and to achieve long-term efficacy. Existing treatments for ovarian cancer, such as chemotherapies and targeted therapies, are unable to cure metastatic disease and prevent tumor relapse. In addition, standard-of-care treatments such as chemotherapy can cause significant morbidity and toxicity.

Provided herein is a transformative new class of immunotherapies for ovarian cancer that is highly specific, effective, and long lasting. This therapeutic strategy, Synthetic Tumor Recruited Immuno-Cellular Therapy (STRICT), leverages tumors themselves to recruit immune cells to destroy the tumors (FIGS. 2A-2B), thereby inducing a strong polyclonal anti-tumor response that should be tunable, safe, long lasting, and effective.

Specifically, we provide synthetic gene circuits that are selectively turned on in ovarian cancer cells only when multiple tumor-specific promoters are active (for example, via digital gene circuits that implement AND logic). These synthetic circuits can be delivered systemically via viral vectors or locally into tumors. We utilize recent advances in synthetic biology to design these synthetic gene circuits to be highly compact, RNA-based (to avoid expressing immunogenic foreign proteins in normal cells), and specifically activated only in ovarian cancer cells (not in any other normal cell type). When activated, these circuits display Surface T-cell Engagers (STEs) and other immunomodulatory molecules, such as checkpoint inhibitors and cytokines, to trigger a robust and targeted anti-tumor immune response. STEs will be designed to engage T-cell receptors on T cells and trigger the T cells to kill the STE-displaying cells. Furthermore, we incorporate safety switches into the gene circuits to enable them to be turned on or off externally.

The first wave of T cells should enact STE-directed killing of tumor cells, followed by secondary waves of polyclonal T cells that target a broader spectrum of cancer antigens released by cell lysis. Thus, the immunotherapy triggered by STRICT may suppress both primary and metastatic tumors, since T cells can provide disseminated immune surveillance throughout the body. Furthermore, these immune responses may enable long-term memory to be established against ovarian cancer. We adapt STRICT to target ovarian adenocarcinoma, the most common and difficult-to treat subset of ovarian cancer that exhibits aggressive behavior and is correlated with poor prognosis (1).

Immunotherapies that harness T-cell effector functions, such as chimeric antigen receptor (CAR) T cells or bispecific T-cell engagers (BiTEs), have achieved potent effects (2, 3). However, the use of these therapies poses significant challenges, especially for solid tumors such as ovarian cancer. Current CAR-T therapy requires custom cell engineering and expansion for every patient, which is expensive and difficult to scale. CAR-T cells need to traffic to tumor sites, target tumor-specific antigens, and persist long-term to mediate robust tumor killing and efficacy (4), which are major challenges for ovarian cancer (5).

BiTEs include of two single-chain variable fragments fused in tandem to enable the engagement and killing of tumor cells by T cells. BiTEs can confer potent and robust tumor killing at concentrations five orders-of-magnitude lower than tumor-targeting antibodies (Abs) (3). However, because BiTEs have short half-lives in vivo (˜2 hours) (6) and solid tumors are generally less accessible to immune cells than hematological malignancies, successful therapy for solid tumors will likely require long periods of continuous i.v. BiTE infusions, which is challenging due to side effects, patient convenience, and therapeutic efficacy. In addition, Surface T-cell Engagers (STEs) have been displayed on cancer cells to recruit T-cell-mediated killing (7-11), but such systems have not been specifically targeted to make systemic therapy possible without significant side effects.

Recently, oncolytic viruses, such as T-Vec, have neared FDA approval to treat melanoma. Oncolytic viruses rely on viral replication to kill tumor cells. However, it can be challenging to engineer oncolytic viruses to only replicate in specific tumor cells and oncolytic viruses have not yet demonstrated good efficacy versus ovarian cancers in clinical trials. In addition, synthetic biologists have developed gene circuits for highly specific intracellular detection of cancer cells based on cancer-specific promoters or microRNA profiles (12, 13). However, synthetic tumor-detecting circuits have only been coupled with intracellular killing mechanisms, which limits their efficacy against cancer because it is virtually impossible to deliver the circuits to 100% of cancer cells.

By harnessing synthetic cancer-detection circuits to command tumor cells to display STEs and to secrete other immunomodulators, we can elicit a robust host immune response to eliminate primary tumor cells and trigger secondary polyclonal T-cell responses. We test whether STRICT can inhibit local lymph node invasion, target systemic metastases, and form immune memory to protect against future relapse. Robust immune responses can be effective against cancer and that synthetic gene circuits can be designed to specifically detect cancer cells with intracellular markers.

We provide at least two methods for target primary, metastatic, and recurring ovarian cancer with STRICT:

1) We provide cancer-detection circuits to command tumor cells to display STEs and secrete immunomodulatory effectors. We validate their therapeutic efficacy in vitro and in vivo. In vitro, we measure T-cell induced cytotoxicity and key cytokines secreted by T cells due to STRICT. In vivo, we determine the minimal number of STE-displaying tumor cells that need to be targeted in order to achieve efficient tumor clearance by STRICT using the ID8 murine model (14).

2) We evaluate STRICT against primary ovarian tumors, metastases, and relapse in mouse models. We use the ID8 murine model to show that metastatic tumors can be eliminated by STRICT and that STRICT can prevent cancer relapse in mice that have survived after initial treatment. Controls to test the efficacy, specificity, and tunability of STRICT include gene circuits that display inactive STEs, gene circuits that are inactive, testing gene circuits in non-cancerous ovarian cells and other normal tissues, and using human versus murine STEs, as well as human versus murine T cells.

This disclosure provides methods for treating ovarian cancer by turning tumors against themselves. STRICT enables long-term activity against ovarian cancer and disseminated T-cell activity against primary and metastatic tumors. Our therapeutic constructs can be customized against a variety of different ovarian cancers, and are easier to scale and deploy in clinical practice versus engineered cell therapies.

STRICT may achieve strong therapeutic effects against primary and metastatic disease, induce long-lasting immune memory, incorporate safety switches, and reduce the cost, labor, and infrastructure needed for therapeutic application. STRICT may be effective against primary and metastatic tumors and achieve long-term protection against tumor relapse. STRICT should overcome limitations of other treatments by enabling convenient, targeted, and safe induction of polyclonal anti-tumor immune responses and long-lasting immune memory from within tumors. STRICT could ultimately replace standard-of-care treatments for ovarian cancer that have toxicities and side effects, and be broadly extensible to other cancers. STRICT is a transformative new treatment modality that can suppress long-term disease by harnessing the immune system against ovarian cancers.

STRICT may be effective against primary and metastatic tumors and achieve long-term protection against tumor relapse. STRICT may be able to replace standard-of-care treatments for ovarian cancer that have limited efficacy and significant toxicities and side effects. Furthermore, this technology establishes a powerful technology platform that can be broadly applied and reprogrammed against a broad range of cancers.

### REFERENCES CITED IN EXAMPLE 7

1. Ries LAG Y J, Keel G E, Eisner M P, Lin Y D, Horner M-J (editors) (2007) SEER Survival Monograph: Cancer Survival Among Adults: U.S. SEER Program, 1988-2001, Patient and Tumor Characteristics. National Cancer Institute, SEER Program, NIH Pub No. 07-6215.
2. Maude S L, Frey N, Shaw P A, Aplenc R, Barrett D M, Bunin N J, Chew A, Gonzalez V E, Zheng Z, Lacey S F, Mahnke Y D, Melenhorst J J, Rheingold S R, Shen A, Teachey D T, Levine B L, June C H, Porter D L, & Grupp S A (2014) Chimeric antigen receptor T cells for sustained remissions in leukemia. N Engl J Med 371(16):1507-1517.
3. Bargou R, Leo E, Zugmaier G, Klinger M, Goebeler M, Knop S, Noppeney R, Viardot A, Hess G, Schuler M, Einsele H, Brandl C, Wolf A, Kirchinger P, Klappers P, Schmidt M, Riethmuller G, Reinhardt C, Baeuerle P A, & Kufer P (2008) Tumor regression in cancer patients by very low doses of a T cell-engaging antibody. Science 321(5891):974-977.
4. Kershaw M H, Westwood J A, Slaney C Y, & Darcy P K (2014) Clinical application of genetically modified T cells in cancer therapy. Clin Trans Immunol 3:e16.
5. Gilham D E, Debets R, Pule M, Hawkins R E, & Abken H (2012) CAR-T cells and solid tumors: tuning T cells to challenge an inveterate foe. Trends Mol Med 18(7):377-384.
6. Klinger M, Brandl C, Zugmaier G, Hijazi Y, Bargou R C, Topp M S, Gokbuget N, Neumann S, Goebeler M, Viardot A, Stelljes M, Bruggemann M, Hoelzer D, Degenhard E, Nagorsen D, Baeuerle P A, Wolf A, & Kufer P (2012) Immunopharmacologic response of patients with B-lineage acute lymphoblastic leukemia to continuous infusion of T cell-engaging CD19/CD3-bispecific BiTE antibody blinatumomab. Blood 119(26):6226-6233.
7. Liao K W, Lo Y C, & Roffler S R (2000) Activation of lymphocytes by anti-CD3 single-chain antibody dimers expressed on the plasma membrane of tumor cells. Gene Ther 7(4):339-347.
8. Paul S, Regulier E, Rooke R, Stoeckel F, Geist M, Homann H, Balloul J M, Villeval D, Poitevin Y, Kieny M P, & Acres R B (2002) Tumor gene therapy by MVA-mediated expression of T-cell-stimulating antibodies. Cancer Gene Ther 9(5):470-477.
9. Liao K W, Chen B M, Liu T B, Tzou S C, Lin Y M, Lin K F, Su C I, & Roffler S R (2003) Stable expression of chimeric anti-CD3 receptors on mammalian cells for stimulation of antitumor immunity. Cancer Gene Ther 10(10):779-790.
10. Yang Z M, Li E M, Lai B C, Wang Y L, & Si L S (2007) Anti-CD3 scFv-B7.1 fusion protein expressed on the surface of HeLa cells provokes potent T-lymphocyte activation and cytotoxicity. Biochem Cell Biol 85(2):196-202.
11. Liu Y, Tuve S, Persson J, Beyer I, Yumul R, Li Z Y, Tragoolpua K, Hellstrom K E, Roffler S, & Lieber A (2011) Adenovirus-mediated intratumoral expression of immunostimulatory proteins in combination with systemic Treg inactivation induces tumor-destructive immune responses in mouse models. Cancer Gene Ther 18(6):407-418.
12. Weber W & Fussenegger M (2012) Emerging biomedical applications of synthetic biology. Nat Rev Genet 13(1):21-35.
13. Nissim L & Bar-Ziv R H (2010) A tunable dual-promoter integrator for targeting of cancer cells. Mol Syst Biol 6:444.
14. Roby K F, Taylor C C, Sweetwood J P, Cheng Y, Pace J L, Tawfik O, Persons D L, Smith P G, & Terranova P F (2000) Development of a syngeneic mouse model for events related to ovarian cancer. Carcinogenesis 21(4):585-591.

### Example 8

Synthetic Tumor Recruited Immuno-Cellular Therapy for Lung Cancer. New therapeutic strategies are needed to treat primary and metastatic lung cancer and to achieve long-term efficacy. Existing treatments for lung cancer, such as chemotherapies and targeted therapies, are unable to cure the disease and prevent tumor relapse. In addition, standard-of-care treatments such as chemotherapy can cause significant morbidity and toxicity.

Provided herein, in some embodiments, are immunotherapies for lung cancer that are be highly specific, effective, and long lasting. This therapeutic strategy, Synthetic Tumor Recruited Immuno-Cellular Therapy (STRICT), leverages tumors themselves to recruit immune cells to destroy the tumors (FIGS. 2A-2B), thereby inducing a strong polyclonal anti-tumor response that should be tunable, safe, long lasting, and effective.

Specifically, we design synthetic gene circuits that are selectively turned on in lung cancer cells only when multiple tumor-specific promoters are active (for example, via digital gene circuits that implement AND logic). These synthetic circuits can be delivered systemically via viral vectors or locally into tumors. We utilize recent advances in synthetic biology to design these synthetic gene circuits to be highly compact, RNA-based (to avoid expressing immunogenic foreign proteins in normal cells), and specifically activated only in lung cancer cells (not in any other normal cell type). When activated, these circuits display Surface T-cell Engagers (STEs) and other immunomodulatory molecules, such as checkpoint inhibitors and cytokines, to trigger a robust and targeted anti-tumor immune response. STEs are designed to engage T-cell receptors on T cells and trigger the T cells to kill the STE-displaying cells. Furthermore, we incorporate safety switches into the gene circuits to enable them to be turned on or off externally.

The first wave of T cells should enact STE-directed killing of tumor cells, followed by secondary waves of polyclonal T cells that target a broader spectrum of cancer antigens released by cell lysis. Thus, the immunotherapy triggered by STRICT may be able to suppress both primary and metastatic tumors, since T cells can provide disseminated immune surveillance throughout the body. Furthermore, these immune responses may enable long-term memory to be established against lung cancer.

We adapt STRICT to target non-small-cell lung cancer (NSCLC), the most common and difficult-to treat subset of lung cancer. STRICT should exhibit efficacy against NSCLC since NSCLC is responsive to some immunotherapies, such as with anti-PD-1 immune checkpoint blockade antibodies (Abs), an immunotherapy that activates host T-cell effector functions (1, 2).

Although anti-PD-1 Abs are approved by the FDA for treating NSCLC, the enhanced survival benefit of anti-PD-1 Abs is only 3.2 months over docetaxel and needs to be further be improved. Other immunotherapies that harness T-cell effector functions, such as chimeric antigen receptor (CAR) T cells or bispecific T-cell engagers (BiTEs), have achieved potent effects against other cancers (3, 4). However, the use of these therapies poses significant challenges for solid tumors such as lung cancer. Current CAR-T therapy requires custom cell engineering and expansion for every patient, which is expensive and difficult to scale. CAR-T cells need to traffic to tumor sites, target tumor-specific antigens, and persist long-term to mediate robust tumor killing and efficacy (5), which are major challenges for lung cancer (6).

BiTEs include of two single-chain variable fragments fused in tandem to enable the engagement and killing of tumor cells by T cells. BiTEs can confer potent and robust tumor killing at concentrations five orders-of-magnitude lower than tumor-targeting Abs (4). However, because BiTEs have short half-lives in vivo (-2 hours) (7) and solid tumors are generally less accessible to immune cells than hematological malignancies, successful therapy for solid tumors will likely require long periods of continuous i.v. BiTE infusions, which is challenging due to side effects, patient convenience, and therapeutic efficacy. In addition, Surface T-cell Engagers (STEs) have been displayed on cancer cells to recruit T-cell-mediated killing (8-12), but such systems have not been specifically targeted to make systemic therapy possible without significant side effects.

Recently, oncolytic viruses that kill tumor cells based on viral replication, such as T-Vec, have neared FDA approval to treat melanoma. However, it can be challenging to engineer oncolytic viruses to only replicate in specific tumor cells and oncolytic viruses have not yet demonstrated good efficacy versus lung cancers in clinical trials. In addition, synthetic biologists have developed gene circuits for highly specific intracellular detection of cancer cells based on cancer-specific promoters or microRNA profiles (13, 14). However, synthetic tumor-detecting circuits have only been coupled with intracellular killing mechanisms, which limits their efficacy against cancer because it is virtually impossible to deliver the circuits to 100% of cancer cells.

By harnessing synthetic cancer-detection circuits to command tumor cells to display STEs and to secrete other immunomodulators, we can elicit a robust host immune response to eliminate primary tumor cells and trigger secondary polyclonal T-cell responses. We show that STRICT can inhibit local lymph node invasion, target systemic metastases, and form immune memory to protect against future relapse. Robust immune responses can be effective against cancer and synthetic gene circuits can be designed to specifically detect cancer cells with intracellular markers.

We provide at least two methods for targeting primary, metastatic, and recurring lung cancer with STRICT:

1) We create cancer-detection circuits to command tumor cells to display STEs and secrete immunomodulatory effectors. We validate their therapeutic efficacy in vitro and in vivo. In vitro, we measure T-cell induced cytotoxicity and key cytokines secreted by T cells due to STRICT. In vivo, we determine the minimal number of STE-displaying tumor cells that need to be targeted in order to achieve efficient tumor clearance by STRICT using the A549 xenograft lung cancer model (15).

2) We evaluate STRICT against primary lung tumors, metastases, and relapse in mouse models. We use the A549 xenograft model and LSL-KrasG12D spontaneous tumor models (16) to show that metastatic tumors can be eliminated by STRICT, and that STRICT can prevent cancer relapse in mice that have survived after initial treatment. Controls to test the efficacy, specificity, and tunability of STRICT include circuits that display inactive STEs, circuits that are inactive, testing circuits in non-cancerous lung cells and other normal tissues, and using human versus murine STEs, and human versus murine T cells.

This disclosure provides methods for treating lung cancer by turning tumors against themselves. Highly specific cancer-detecting circuits have not yet been integrated with immunotherapy against lung cancer. STRICT should enable long-term activity against lung cancer and disseminated T-cell activity against primary and metastatic tumors. Our therapeutic constructs can be customized against a variety of different lung cancers, and should be easier to scale and deploy in clinical practice versus engineered cell therapies.

STRICT can achieve strong therapeutic effects against primary and metastatic disease, induce long-lasting immune memory, incorporate safety switches, and reduce the cost, labor, and infrastructure needed for therapeutic application. STRICT should be effective against primary and metastatic tumors and achieve long-term protection against tumor relapse. STRICT should overcome limitations of other treatments by enabling convenient, targeted, and safe induction of polyclonal anti-tumor immune responses and long-lasting immune memory from within tumors. STRICT may ultimately replace standard-of-care treatments for lung cancer that have toxicities and side effects, and be broadly extensible to other cancers. Here, we aim to how that STRICT is a transformative new treatment modality that may suppress long-term disease by harnessing the immune system against lung cancers.

This disclosure provides a powerful technology platform that can be broadly applied and reprogrammed against a broad range of cancers, including lung cancer.

### REFERENCES CITED IN EXAMPLE 8

1. S. L. Topalian et al., Safety, activity, and immune correlates of anti-PD-1 antibody in cancer. N Engl J Med 366, 2443-2454 (2012).
2. J. R. B. Scott N. Gettinger, Naiyer A. Rizvi, Neal Ready, Laura Quan Man Chow, Scott J. Antonia, Marc E. Buyse, Jacek Jassem, Friedrich Graf Finckenstein, Lucio Crinò, Thomas James Lynch, A phase III comparative study of nivolumab (anti-PD-1; BMS-963558; ONO-4538) versus docetaxel in patients (pts) with previously treated advanced/metastatic nonsquamous non-small-cell lung cancer (NSCLC). J Clin Oncol, suppl; abstr TPS8121 (2013).
3. S. L. Maude et al., Chimeric antigen receptor T cells for sustained remissions in leukemia. N Engl J Med 371, 1507-1517 (2014).
4. R. Bargou et al., Tumor regression in cancer patients by very low doses of a T cell-engaging antibody. Science 321, 974-977 (2008).
5. M. H. Kershaw, J. A. Westwood, C. Y. Slaney, P. K. Darcy, Clinical application of genetically modified T cells in cancer therapy. Clin Trans Immunol 3, e16 (2014).
6. D. E. Gilham, R. Debets, M. Pule, R. E. Hawkins, H. Abken, CAR-T cells and solid tumors: tuning T cells to challenge an inveterate foe. Trends Mol Med 18, 377-384 (2012).
7. M. Klinger et al., Immunopharmacologic response of patients with B-lineage acute lymphoblastic leukemia to continuous infusion of T cell-engaging CD19/CD3-bispecific BiTE antibody blinatumomab. Blood 119, 6226-6233 (2012).
8. K. W. Liao, Y. C. Lo, S. R. Roffler, Activation of lymphocytes by anti-CD3 single-chain antibody dimers expressed on the plasma membrane of tumor cells. Gene Ther 7, 339-347 (2000).
9. S. Paul et al., Tumor gene therapy by MVA-mediated expression of T-cell-stimulating antibodies. Cancer Gene Ther 9, 470-477 (2002).
10. K. W. Liao et al., Stable expression of chimeric anti-CD3 receptors on mammalian cells for stimulation of antitumor immunity. Cancer Gene Ther 10, 779-790 (2003).
11. Z. M. Yang, E. M. Li, B. C. Lai, Y. L. Wang, L. S. Si, Anti-CD3 scFv-B7.1 fusion protein expressed on the surface of HeLa cells provokes potent T-lymphocyte activation and cytotoxicity. Biochem Cell Biol 85, 196-202 (2007).
12. Y. Liu et al., Adenovirus-mediated intratumoral expression of immunostimulatory proteins in combination with systemic Treg inactivation induces tumor-destructive immune responses in mouse models. Cancer Gene Ther 18, 407-418 (2011).
13. W. Weber, M. Fussenegger, Emerging biomedical applications of synthetic biology. Nat Rev Genet 13, 21-35 (2012).
14. L. Nissim, R. H. Bar-Ziv, A tunable dual-promoter integrator for targeting of cancer cells. Mol Syst Biol 6, 444 (2010).
15. K. Kondo et al., Orthotopically implanted SCID mouse model of human lung cancer suitable for investigating metastatic potential and anticancer drug effects. Oncol Rep 12, 991-999 (2004).
16. M. C. Kwon, A. Berns, Mouse models for lung cancer. Mol Oncol 7, 165-177 (2013).

### Example 9

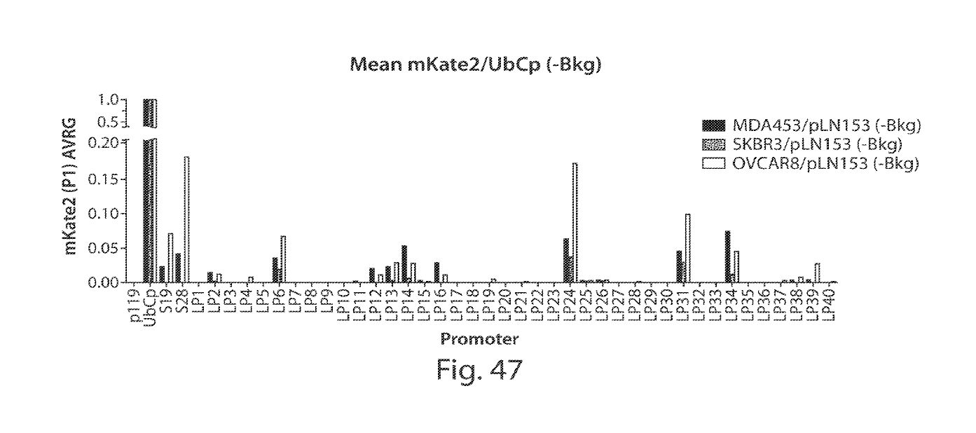

Synthetic Promoters Library. Provided herein, in some embodiments, is a simple, fast and cost-efficient method to characterize the post translational regulation of transcription factors. The methods may be used, for example, to identify highly specific and very short synthetic promoters that can be used to target a cell state of interest, which is important both for research and personalized medicine. This may be done, for example, by identifying highly specific binding motifs which are activated in a specific cell state. Current methods such as RNA-Seq and ChIP-Seq can be misleading, since RNA levels are not always correlated with protein activity (p53 is a great example) and binding of TFs to the DNA is not always correlated with transcriptional activation (for example, the TF can function as a repressor). The method of the present disclosure, in some embodiments, provides direct evidence of the binding motifs which are activated in specific cell state and the activation levels of these motifs. The Bioinformatics layer enables characterizing the transcription factors associated with these motifs and therefore deciphering the transcriptional cascades activated in the cell state of interest. For FIGS. 46 and 47, synthetic promoters were isolated from NB508-low library.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An engineered genetic circuit, comprising:
(a) a first nucleic acid comprising a promoter operably linked to (i) a nucleotide sequence encoding an output messenger RNA (mRNA) containing an intronic microRNA (miRNA) and (ii) a nucleotide sequence encoding at least one miRNA binding site complementary to the miRNA of (a)(i); and
(b) a second nucleic acid comprising a promoter operably linked to a nucleotide sequence encoding at least one miRNA binding site complementary to the miRNA of (a)(i).

2. The engineered genetic circuit of claim 1, wherein output mRNA encodes a synthetic T cell engager (STE) or a bispecific T cell engager (BiTE).

3. The engineered genetic circuit of claim 1, wherein the output mRNA encodes an output protein that binds to a T cell surface marker.

4. The engineered genetic circuit of claim 3, wherein the T cell surface marker is CD3, CD4, CD8 or CD45.

5. The engineered genetic circuit of any one of claims 1-4, wherein the output protein is an antibody or antibody fragment that binds specifically to the T cell surface antigen.

6. The engineered genetic circuit of any one of claims 1-5, wherein the output mRNA encodes an anti-cancer agent.

7. The engineered genetic circuit of claim 6, wherein the anti-cancer agent is a chemokine, a cytokine or a checkpoint inhibitor.

8. The engineered genetic circuit of any one of claims 1-7, wherein the promoter of (a) and/or (b) is an inducible promoter.

9. The engineered genetic circuit of claim 8, wherein the promoter of (a) and/or (b) is a tumor-specific promoter or a cancer-promoter.

10. The engineered genetic circuit of claim 9, wherein the promoter of (a) and/or (b) is SSX1 or H2A1.

11. The engineered genetic circuit of any one of claims 1-10, wherein the nucleotide sequence of (a)(ii) encodes 2-5 miRNA binding sites complementary to the miRNA of (a)(i)

12. The engineered genetic circuit of any one of claims 1-11, wherein the nucleotide sequence of (b) encodes 2-10 miRNA binding sites complementary to the miRNA of (a)(i)

13. The engineered genetic circuit of any one of claims 1-12, wherein the output protein is a transcription factor.

14. The engineered genetic circuit of claim 13, further comprising at least one nucleic acid comprising a promoter operably linked to a nucleic acid encoding an output nucleic acid or an output protein.

15. The engineered genetic circuit of claim 14, wherein the output mRNA encodes a transcription factor that can bind to and activate transcription of the promoter of the at least one nucleic acid.

16. The engineered genetic circuit of any one of claims 1-15, further comprising a nucleic acid comprising a promoter operably linked to (i) a nucleotide sequence encoding an additional output messenger RNA (mRNA) containing an intronic microRNA (miRNA) and (ii) a nucleotide sequence encoding at least one miRNA binding site complementary to the miRNA of (a)(i), wherein the additional output mRNA encodes a chemokine, a cytokine, a checkpoint inhibitor or a combination thereof.

17. A cell comprising at least one engineered genetic circuit of any one of claims 1-16.

18. The cell of claim 17, wherein the cell is a tumor cell.

19. A method, comprising administering to a subject having a tumor at least one engineered genetic circuit of any one of claims 1-15.

20. The method of claim 19, wherein the subject has ovarian cancer, breast cancer or lung cancer.

21. The method of claim 19 or 20, wherein the engineered genetic circuit is administered systemically to the subject.

22. The method of any one of claims 19-21, wherein the engineered genetic circuit is delivered using a viral delivery system.

23. The method of claim 22, wherein the viral delivery system is a lentiviral delivery system, an adenoviral delivery system or an adeno-associated viral delivery system.

24. The method of any one of claims 19-21, wherein the engineered genetic circuit is delivered using a non-viral delivery system.

25. The method of claim 19 or 20, wherein the engineered genetic circuit is administered locally to the tumor of the subject.

26. The method of claim 25, wherein the engineered genetic circuit is administered locally to the tumor using a hydrogel-based delivery system.

27. The method of any one of claims 19-26, wherein the output mRNA of at least one of the engineered genetic circuits encodes an output protein that binds to a T cell surface marker, and the output mRNA of at least one other engineered genetic circuit encodes a chemokine, a cytokine or a checkpoint inhibitor.

28. A composition comprising an anti-CD3e scFv antibody fragment fused with an transmembrane protein.

29. The composition of claim 28, wherein the transmembrane protein comprises cytoplasmic truncated Duffy Antigen/Receptor for Chemokines (DARC).

30. A composition comprising an anti-CD3e scFv antibody fragment fused with a human IgG1-Hinge-CH2-CH3 domain, a murine B7.1-transmembrane and a cytoplasmic domain

31. An engineered genetic circuit, comprising:
(a) a first nucleic acid comprising a first tumor-specific promoter operably linked to (i) a nucleotide sequence encoding an output messenger RNA (mRNA) containing an intronic microRNA (miRNA) and (ii) a nucleotide sequence encoding at least one miRNA binding site complementary to the miRNA of (a)(i), wherein the output mRNA encodes a synthetic T cell engager or a bispecific T cell engager; and
(b) a second nucleic acid comprising a second promoter different from the first promoter and operably linked to a nucleotide sequence encoding at least one miRNA binding site complementary to the miRNA of (a)(i).

32. The engineered genetic circuit of claim 31 further comprising a nucleic acid comprising a tumor-specific promoter operably linked to (i) a nucleotide sequence encoding an additional output messenger RNA (mRNA) containing an intronic microRNA (miRNA) and (ii) a nucleotide sequence encoding at least one miRNA binding site complementary to the miRNA of (a)(i), wherein the additional output mRNA encodes a chemokine, a cytokine, a checkpoint inhibitor or a combination thereof.

33. An engineered genetic circuit, comprising:
(a) a first nucleic acid comprising a promoter operably linked to
(i) a nucleotide sequence encoding an output messenger RNA (mRNA) containing an intronic microRNA (miRNA),
(ii) a nucleotide sequence encoding an intronic miRNA, and
(iii) a nucleotide sequence encoding a miRNA binding site (miRNA-BS);
(b) a second nucleic acid comprising a promoter operably linked to
(i) a nucleotide sequence encoding an output mRNA containing an intronic miRNA,
(ii) a nucleotide sequence encoding an intronic miRNA, and
(iii) a nucleotide sequence encoding a miRNA-BS; and
(c) a third nucleic acid comprising a promoter operably linked to a nucleotide sequence encoding an output protein linked to a miRNA-BS,
wherein the miRNA-BS of (a)(iii) is complementary to the miRNA of (b)(i), the miRNA-BS of (b)(iii) is complementary to the miRNA of (a)(i), and the miRNA-BS of (c) is complementary to the miRNA of (a)(ii) and the miRNA of (b)(ii).

34. An engineered genetic circuit, comprising:
(a) a first nucleic acid comprising a promoter operably linked to
(i) a nucleotide sequence encoding a nascent RNA transcript containing an intronic microRNA (miRNA), and
(ii) a nucleotide sequence encoding at least one miRNA binding site (miRNA-BS);
(b) a second nucleic acid comprising a promoter operably linked to
(i) a nucleotide sequence encoding a nascent RNA transcript containing an intronic miRNA, and
(ii) a nucleotide sequence encoding at least one miRNA-BS; and
(c) a third nucleic acid comprising a promoter operably linked to a nucleic acid encoding an output protein linked to (i) a first miRNA-BS and (ii) a second miRNA-BS,
wherein the at least one miRNA-BS of (a)(ii) is complementary to the miRNA of (b)(i), the at least one miRNA-BS of (b)(ii) is complementary to the miRNA of (a)(i), the first miRNA-BS of (c)(i) is complementary to the miRNA of (a)(i), and the second miRNA-BS of (c)(ii) is complementary to the miRNA of (b)(i).

35. An engineered genetic circuit, comprising:
(a) a first nucleic acid comprising a promoter operably linked to a nucleotide sequence encoding a nascent RNA transcript containing an intronic microRNA (miRNA);
(b) a second nucleic acid comprising a promoter operably linked to a nucleotide sequence encoding a nascent RNA transcript containing an intronic miRNA; and
(c) a third nucleic acid comprising a promoter operable linked to a nucleotide sequence encoding an output protein linked to (i) a first miRNA-BS and (ii) a second miRNA-BS,
wherein the first miRNA-BS of (c)(i) is complementary to the miRNA of (a), and the second miRNA-BS of (c)(ii) is complementary to the miRNA of (b).

36. An engineered genetic circuit, comprising:
(a) a first nucleic acid comprising a promoter operably linked to a nucleotide sequence encoding a nascent RNA transcript containing an intronic microRNA (miRNA); and
(b) a second nucleic acid comprising a promoter operably linked to a nucleotide sequence encoding an output protein linked to a miRNA binding site (miRNA-BS);
wherein the miRNA-BS of (b) is complementary to the miRNA of (a).

37. An engineered genetic circuit, comprising:
(a) a first nucleic acid comprising a promoter operably linked to (i) a nucleotide sequence encoding an output messenger RNA (mRNA) containing an intronic microRNA (miRNA) and (ii) at least one miRNA binding site (miRNA-BS); and
(b) a second nucleic acid comprising a promoter operably linked to (i) a nucleotide sequence encoding an output mRNA containing an intronic miRNA and (ii) at least one miRNA-BS,
wherein the at least one miRNA-BS of (a) is complementary to the miRNA of (b), the at least one miRNA-BS of (b) is complementary to the miRNA of (a).

38. An engineered genetic circuit, comprising:
(a) a first nucleic acid comprising a promoter operably linked to a nucleotide sequence encoding a nascent RNA transcript containing an intronic microRNA (miRNA);
(b) a second nucleic acid comprising a promoter operably linked to a nucleotide sequence encoding an output protein; and
(c) a third nucleic acid comprising a promoter operable linked to a nucleotide sequence encoding an output protein linked to an miRNA binding site,
wherein the miRNA-BS of (c) is complementary to the miRNA of (a).

39. An engineered genetic circuit, comprising:
(a) a first nucleic acid comprising a promoter operably linked to a nucleotide sequence encoding an output protein linked to a microRNA binding site (miRNA-BS); and
(b) a second nucleic acid comprising a promoter operably linked to a nucleotide sequence encoding a nascent RNA transcript containing an intronic miRNA,
wherein the miRNA-BS of (a) is complementary to the miRNA of (b).

40. A synthetic promoter library comprising a plurality of nucleic acids, wherein each nucleic acid comprises a promoter sequence having at least two 8 mer nucleotide sequences in tandem without any spacer nucleotides between each 8 mer nucleotide sequence.

41. The synthetic promoter library of claim 40, wherein each nucleic acid comprises at least six 8 mer nucleotide sequences in tandem without any spacer nucleotides between each 8 mer nucleotide sequence.

42. The synthetic promoter library of claim 40 or claim 41, wherein each nucleic acid comprises at least twelve 8 mer nucleotide sequences in tandem without any spacer nucleotides between each 8 mer nucleotide sequence.

43. The synthetic promoter library of any one of claims 40-42, wherein the 8 mer nucleotide sequence is NNNNNNNN, wherein each N represents any nucleotide.

44. The synthetic promoter library of any one of claims 40-43, wherein each of the nucleic acids further comprises a restriction endonuclease site at the 5′ and 3′ ends.

45. The synthetic promoter library of claim 44, wherein the restriction endonuclease site at the 5′ end is a SbfI site and the restriction endonuclease site at the 3′ end is an AscI site.

46. The synthetic promoter library of any one of claims 40-45, wherein each of the nucleic acids further comprises a nucleotide sequence encoding an output molecule operably linked to the promoter sequence.

47. The synthetic promoter library of claim 46, wherein the output molecule is a detectable molecule.

48. A synthetic promoter library comprising a plurality of nucleic acids, wherein each nucleic acid comprises a promoter sequence having at least two 8 mer nucleotide sequences in tandem with a 3 mer nucleotide spacer between each 8 mer nucleotide sequence.

49. The synthetic promoter library of claim 48, wherein each nucleic acid comprises at least six 8 mer nucleotide sequences in tandem with a 3 mer nucleotide spacer between each 8 mer nucleotide sequence.

50. The synthetic promoter library of claim 48 or claim 49, wherein each nucleic acid comprises at least nine 8 mer nucleotide sequences in tandem with a 3 mer nucleotide spacer between each 8 mer nucleotide sequence.

51. The synthetic promoter library of any one of claims 48-50, wherein the 8 mer nucleotide sequence is NNNNNNNN, wherein each N represents any nucleotide.

52. The synthetic promoter library of any one of claims 48-51, wherein each of the nucleic acids further comprises a restriction endonuclease site at the 5′ and 3′ ends.

53. The synthetic promoter library of claims 52, wherein the restriction endonuclease site at the 5′ end is a SbfI site and the restriction endonuclease site at the 3′ end is an AscI site.

54. The synthetic promoter library of any one of claims 48-53, wherein the 3 mer nucleotide spacers are selected from AGC, ATC, GAC, ACT, AGT, GTC, GAT, and GCT.

55. The synthetic promoter library of claim 54, wherein each 3 mer nucleotide spacer is different.

56. The synthetic promoter library of any one of claims 48-55, wherein each of the nucleic acids further comprises a nucleotide sequence encoding an output molecule operably linked to the promoter sequence.

57. The synthetic promoter library of claim 56, wherein the output molecule is a detectable molecule.

58. A synthetic promoter library comprising a plurality of nucleic acids, wherein each nucleic acid comprises a promoter sequence having at least two 11 mer nucleotide sequences in tandem with a 3 mer nucleotide spacer between each 11 mer nucleotide sequence.

59. The synthetic promoter library of claim 58, wherein each nucleic acid comprises at least four 11 mer nucleotide sequences in tandem with a 3 mer nucleotide spacer between each 11 mer nucleotide sequence.

60. The synthetic promoter library of claim 58 or claim 59, wherein each nucleic acid comprises at least seven 11 mer nucleotide sequences in tandem with a 3 mer nucleotide spacer between each 11 mer nucleotide sequence.

61. The synthetic promoter library of any one of claims 58-60, wherein the 11 mer nucleotide sequence is NNNNNNNNNNN, wherein each N represents any nucleotide.

62. The synthetic promoter library of any one of claims 58-61, wherein each of the nucleic acids further comprises a restriction endonuclease site at the 5′ and 3′ ends.

63. The synthetic promoter library of claims 62, wherein the restriction endonuclease site at the 5′ end is a SbfI site and the restriction endonuclease site at the 3′ end is an AscI site.

64. The synthetic promoter library of any one of claims 58-63, wherein the 3 mer nucleotide spacers are selected from AGC, ATC, GAC, ACT, AGT, GTC, GAT, and GCT.

65. The synthetic promoter library of claim 64, wherein each 3 mer nucleotide spacer is different.

66. The synthetic promoter library of any one of claims 58-65, wherein each of the nucleic acids further comprises a nucleotide sequence encoding an output molecule operably linked to the promoter sequence.

67. The synthetic promoter library of claim 66, wherein the output molecule is a detectable molecule.

68. A method of selecting a synthetic promoters comprising
obtaining a library comprising nucleic acid molecules comprising synthetic promoter sequences operably linked to an output molecule,
expressing the library in one or more types of cells,
detecting the expression of the output molecule, and
isolating the cells in which the output molecule is expressed.

69. The method of claim 68, further comprising
determining the sequence of the synthetic promoter sequences in the isolated cells.

70. The method of claim 68 or claim 69, wherein the one or more types of cells are at least two different types of cells.

71. The method of claim 70, further comprising comparing the synthetic promoter sequences that drive the expression of the output molecule in each of the at least two different types of cells to identify synthetic promoter sequences that are more active in one of the at least two different types of cells than in another of the at least two different types of cells.

72. The method of claim 71, wherein the at least two different types of cells are cancer cells and non-cancer cells, and wherein promoters are identified that are more active in cancer cells than in non-cancer cells.
